SEQUENCE LISTING (14 sequences)

SEQ ID NO: 1 - DNA, 19 bp, synthetic construct
  source 1..19: mol_type = other DNA
gccgaagggtttcgaggcc

SEQ ID NO: 2 - DNA, 19 bp, synthetic construct
  source 1..19: mol_type = other DNA
gccgaagggcggcgaggcc

SEQ ID NO: 3 - DNA, 19 bp, synthetic construct
  source 1..19: mol_type = other DNA
cggcaagggtttcgtcgag

SEQ ID NO: 4 - DNA, 19 bp, synthetic construct
  source 1..19: mol_type = other DNA
cggcaagggcggcgtcgag

SEQ ID NO: 5 - DNA, 16 bp, synthetic construct
  source 1..16: mol_type = other DNA
cggcaagggcgtcgag

SEQ ID NO: 6 - DNA, 19 bp, synthetic construct
  source 1..19: mol_type = other DNA
  misc_feature 1..19: note = Topoisomerase Charged Oligonucleotide
gccgggcctcgaaaccctt

SEQ ID NO: 7 - DNA, 19 bp, synthetic construct
  source 1..19: mol_type = other DNA
  misc_feature 1..19: note = Topoisomerase Charged Oligonucleotide
gccgggcctcgccgccctt

SEQ ID NO: 8 - DNA, 19 bp, synthetic construct
  source 1..19: mol_type = other DNA
  misc_feature 1..19: note = Topoisomerase Charged Oligonucleotide
cggcctcgacgaaaccctt

SEQ ID NO: 9 - DNA, 19 bp, synthetic construct
  source 1..19: mol_type = other DNA
  misc_feature 1..19: note = Topoisomerase Charged Oligonucleotide
cggcctcgacgccgccctt

SEQ ID NO: 10 - DNA, 16 bp, synthetic construct
  source 1..16: mol_type = other DNA
  misc_feature 1..16: note = Topoisomerase Charged Oligonucleotide
cggcctcgacgccctt

SEQ ID NO: 11 - DNA, 18 bp, synthetic construct
  source 1..18: mol_type = other DNA
  misc_feature 1..18: note = Topoisomerase Charged Oligonucleotide
gccgcttgaaacccttcg

SEQ ID NO: 12 - DNA, 16 bp, synthetic construct
  source 1..16: mol_type = other DNA
gccgaagggtttcaag

SEQ ID NO: 13 - DNA, 40 bp, synthetic construct
  source 1..40: mol_type = other DNA
cggcaatctgcacgttaatatcgcaggaattcgtcagcag

SEQ ID NO: 14 - DNA, 36 bp, synthetic construct
  source 1..36: mol_type = other DNA
ctgctgacgaattcctgcgatattaacgtgcagatt

(19) United States
(12) Patent Application Publication
(10) Pub. No.: US 2024/0308259 A1
(43) Pub. Date: Sep. 19, 2024

(54) SYSTEMS AND METHODS FOR WRITING DATA STORED IN A POLYMER USING INKJET DROPLETS

(71) Applicant: Iridia, Inc., Carlsbad, CA (US)

(72) Inventors: Thomas Henry CAULEY, III, Carlsbad, CA (US); Kelsey SCHRAMMA, Carlsbad, CA (US); Paul F. PREDKI, Carlsbad, CA (US); Melania STRYCHARSKA, Carlsbad, CA (US)

(21) Appl. No.: 18/444,662

(22) Filed: Feb. 17, 2024

Related U.S. Application Data
(60) Provisional application No. 63/485,832, filed on Feb. 17, 2023.

(51) Int. Cl. B41M 5/00; B08B 3/04; B41M 3/00; C12P 19/34

(52) U.S. Cl. CPC B41M 5/0047; B08B 3/04; B41M 3/008; B41M 5/0011; C12P 19/34

(57) ABSTRACT

The disclosure provides a novel system and methods for writing, by at least one ink jet print head, e.g., piezo electric print head, a unique code in polymer memory strands dispensed on at least one writing spot on a wafer array, and reagents and materials useful therein.

### CROSS-REFERENCE TO RELATED APPLICATIONS

This application claims priority to U.S. Provisional Application No. 63/485,832, filed Feb. 17, 2023, the entire contents of which are incorporated herein by reference.

### REFERENCE TO A SEQUENCE LISTING

The instant application contains a Sequence Listing which has been submitted electronically in xml (ST.26) format (DNA-09-USP_ST.26_SequenceListing.xml; Size: 15,862 bytes; and Date of Creation: Jun. 5, 2024), the contents of which are herein incorporated by reference in their entirety.

### FIELD

The invention relates to novel methods and systems for information storage using DNA sequences.

### BACKGROUND

There is a continuing demand to store ever more data on or in physical media, with storage devices getting ever smaller as their capacity gets bigger. The amount of data stored is reportedly doubling in size every two years, and according to one study, by 2020 the amount of data we create and copy annually will reach 44 zettabytes, or 44 trillion gigabytes. Moreover, existing data storage media such as hard drives, optical media, and magnetic tapes, are relatively unstable and become corrupted after prolonged storage.

There is an urgent need for alternative approaches to storing large volumes of data for extended periods, e.g., decades or centuries.

### BRIEF DESCRIPTION OF THE DRAWINGS

The present invention will become more fully understood from the detailed description and the accompanying drawings, wherein:

FIG. 1 is a top view of a silicon wafer with spot patterns, in accordance with embodiments of the present disclosure.

FIG. 2 is a side view of a silicon wafer with patterned layers for writing coded DNA on a surface, in accordance with embodiments of the present disclosure.

FIG. 3 is a cross-section side view showing a fabrication process to create patterned wafers, in accordance with embodiments of the present disclosure.

FIG. 4 is a diagram showing an HfO2 layer for attachment of DNA molecules to the wafer, in accordance with embodiments of the present disclosure.

FIG. 5 is a top view of a portion of two wafers with different spot pattern sizes and their relation to FIG. 2, in accordance with embodiments of the present disclosure.

FIG. 6 is a side view of a silicon wafer with patterned layers and showing three nozzles of a piezo-electric print head and different stages of a released droplet as it travels toward the upper surface of the patterned wafer, in accordance with embodiments of the present disclosure.

FIG. 7 is a side view of a silicon wafer with patterned layers showing how the droplets change geometry after contacting the pillar of a patterned silica surface, three different droplet sizes, in accordance with embodiments of the present disclosure.

FIG. 8A is a side view of a silicon wafer with patterned layers showing starter DNA strands in liquid attached to pillars, showing a side view of a print head bank having three or four nozzles, and showing that a wash cycle may be done horizontally across the wafter surface or vertically as part of the print head, in accordance with embodiments of the present disclosure.

FIG. 8B is a side view of a silicon wafer with patterned layers showing starter DNA strands in liquid attached to pillars, showing a side view of a print head bank having four of five nozzles for 2-bit binary encoding, and showing that a wash cycle may be done horizontally across the wafter surface or vertically as part of the print head, in accordance with embodiments of the present disclosure.

FIG. 9A is a side view of a silicon wafer with patterned layers showing starter DNA strands in liquid attached to pillars and showing a side view of a data writing (or printing) process to add bits or codes to the free end of starter DNA strands on the wafer, in accordance with embodiments of the present disclosure.

FIG. 9B is a side view of a silicon wafer with patterned layers showing starter DNA strands in liquid attached to pillars and showing a side view of a data writing (or printing) process to add bits or codes to the free end of starter DNA strands on the wafer for 2-bit binary encoding, in accordance with embodiments of the present disclosure.

FIG. 10 is a side view of a silicon wafer with patterned layers showing starter DNA strands attached to pillars at one end and attached to coded DNA on the other end, and also showing how a cleaving fluid may be used to remove the coded DNA strands from the wafer, in accordance with embodiments of the present disclosure.

FIG. 11A is a diagram showing an example of a plurality of spots with coded DNA (after writing codes) attached to a wafer and a process for removing, storing and reading/decoding the data, in accordance with embodiments of the present disclosure.

FIG. 11B is a flow diagram for decoding polymer memory string data, in accordance with embodiments of the present disclosure.

FIG. 12 is a side view of a silicon wafer with patterned layers showing a multi nozzle print head releasing droplets of starter DNA onto the pillars of the wafer, and showing an optional preparation fluid to prepare the wafer for starter DNA attachment, in accordance with embodiments of the present disclosure.

FIG. 13A is a side view of a silicon wafer with patterned layers showing starter DNA strands in liquid attached to pillars, showing a side view of an array of print head banks each having three nozzles for printing to a plurality of spots simultaneously, in accordance with embodiments of the present disclosure.

FIG. 13B is the diagram of FIG. 13A where the three nozzle print head banks are replaced by five nozzle print head banks, for printing to a plurality of spots simultaneously, in accordance with embodiments of the present disclosure.

FIG. 14 is a diagram of a dual print head assembly writing process, showing an entire row and layer of 1's and 0's being written followed by an adapter layer being written, followed by another layer of 1's and 0's, in accordance with embodiments of the present disclosure.

FIG. 15 shows an example of an inkjet printer showing motion axes, a computer controller, and a zoom in of a wafer for printing DNA, in accordance with embodiments of the present disclosure.

FIG. 16A show images of printing drops of various sizes for a 50 micron diameter active spots, in accordance with embodiments of the present disclosure.

FIG. 16B and FIG. 16C show images of results of printing and washing five times for 50 um diameter active wafer spots, 100 dpi apart, in accordance with embodiments of the present disclosure.

FIG. 16D shows images of results of printing and washing five times for 50 um diameter active wafer spots, 100 dpi apart, in accordance with embodiments of the present disclosure.

FIG. 17A shows an image of the wafer and two graphs showing mass spec topogation results from HIDI release after writing, in accordance with embodiments of the present disclosure.

FIG. 17B shows an image of the wafer after drying out for three weeks and two graphs showing mass spectrometry results from formamide release from dried wafer, in accordance with embodiments of the present disclosure.

FIG. 18A shows two different data format listings of the bits on a memory string, in accordance with embodiments of the present invention.

FIG. 18B shows a data format listing of the bits on a memory string for each of the spots on the wafer in the array, in accordance with embodiments of the present disclosure.

FIG. 19A is a block diagram showing an inkjet printing system showing print head control and wafer array/stage control logic and an instrument for fluidics/reagents, in accordance with embodiments of the present disclosure.

FIG. 19B is a block diagram of the computer system of FIG. 19A, in accordance with embodiments of the present disclosure.

FIG. 20A is a flow diagram for performing loading starter DNA, writing (printing) and unloading coded polymers in an inkjet writing system, in accordance with embodiments of the present disclosure.

FIG. 20B is a flow diagram for performing writing (printing) a bit or code to a polymer in an inkjet writing system, in accordance with embodiments of the present disclosure.

FIG. 21 shows a scheme of phosphatase-free “bit addition” or “topogation”.

FIG. 22 shows a scheme of the phosphatase-free topoisomerase-mediated synthesis of DNA polymer storing binary information “10110”.

FIG. 23A is a side cross-section view of a patterned wafer substrate having a hydrophobic coating and showing a process for using silanization to create the hydrophobic coating, in accordance with embodiments of the present disclosure.

FIG. 23B is a side view of patterned wafers with two different hydrophobic silane coatings (PFOTES above the dashed line, DDTMS below the dashed line), shown at 3 different temperatures, in accordance with embodiments of the present disclosure.

FIG. 24A shows images for an example of topoisomerase-based ink printed on a glass slide with two different resolutions, in accordance with embodiments of the present disclosure.

FIG. 24B is a graph for the example of FIG. 24A of conversion % vs time, comparing ligation kinetic performance of jetted and unjetted topoisomerase-based ink at four different time points, in accordance with embodiments of the present disclosure.

FIG. 25A shows an image for another example of topoisomerase-based ink printed on a glass slide with 300 dpi resolution, in accordance with embodiments of the present disclosure.

FIG. 25B is a graph for the example of FIG. 25A of conversion % vs time, comparing ligation kinetic performance of jetted and unjetted topoisomerase-based ink at four different time points, and a blown-up comparison at a 20 second time point, in accordance with embodiments of the present disclosure.

FIG. 26A is a diagram showing an example of bonding and linking chemistry for printing a patterned wafer, suitable for receiving topoisomerase ink, with and without a hydrophobic coating, in accordance with embodiments of the present disclosure.

FIG. 26B shows consecutive images from a cycle of five prints of topoisomerase ink on a patterned wafer for the example of FIG. 26A, in accordance with embodiments of the present disclosure.

FIG. 27A is a side view of a flat substrate with pattern spots with acceptors for synthesis, the substrate moving under stationary inkjet head in a first direction, in accordance with embodiments of the present invention.

FIG. 27B is a side view of a flat substrate of FIG. 27A, the substrate moving under stationary inkjet head in a reverse direction from that of FIG. 27A, in accordance with embodiments of the present invention.

FIG. 27C is a side view of the substrate of FIG. 27A showing details of cell (or spot) sizes, dimensions, and acceptor density, in accordance with embodiments of the present invention.

FIG. 27D is a side view of the substrate of FIG. 27C showing a process for a topogation reaction on the surface of the cell (or spot) on the substrate, in accordance with embodiments of the present invention.

FIG. 27E is a side view of steps for an embodiment of a wash cycle and/or wash/deblock cycle after a topogation reaction, in accordance with embodiments of the present invention.

FIG. 28A is a side view of a conveyor track, linear actuator, inkjet writing system for moving (or shuttling) portable inkjet-writable wafers (or shuttles), e.g., via an electromagnetic track and queue elevators, through inkjet head writing stations and washing stations, including loading and unloading shuttles or coded polymers, in accordance with embodiments of the present invention.

FIG. 28B is a perspective view of the portable inkjet-writable silicon wafers of FIG. 28A, and a blow-up of hexagonal fluidic wells within the wafer, in accordance with embodiments of the present invention.

FIG. 29 is a perspective view of a stacked rotary disk (or platen) based turntable inkjet writing system for inkjet-writable silicon wafer disks, where the outer edges of the discs are rotated through inkjet head writing stations and washing stations, in accordance with embodiments of the present invention.

FIG. 30A is a perspective view of a vacuum manifold plate that sucks fluid out of each well in a microwell plate and into a manifold and out an exit or drain port and then a drain system to expedite the wash cycle of a wafer, in accordance with embodiments of the present invention.

FIG. 30B shows three perspective cut-away views of the vacuum manifold plate of FIG. 30A and shows the path for fluid injection into the wells and extraction from the wells, with two close-up views of same, in accordance with embodiments of the present invention.

FIG. 30C shows a perspective view of the vacuum manifold of FIG. 30B showing vacuum needles and a blown-up view showing the needles, and a perspective cutaway view of an inkjet writing assembly having a vacuum manifold, in accordance with embodiments of the present invention.

FIG. 31 is a cross-section side view showing an alternative fabrication process to create patterned wafers, in accordance with embodiments of the present disclosure.

FIG. 32 is a diagram of a dual print head assembly writing process using a print/puddle approach using two print heads, showing two writing cycles and four layers of 1's and 0's being written and corresponding written codes, in accordance with embodiments of the present disclosure.

### DETAILED DESCRIPTION

The following description of the preferred embodiment(s) is merely exemplary in nature and is in no way intended to limit the invention, its application, or uses.

The following commonly-owned issued patents contain subject matter related to that described herein, each of which are hereby incorporated by reference in their entirety to the fullest extent permitted by applicable law: U.S. Pat. Nos. 10,438,662 and 10,640,822. The aforementioned commonly-owned patents discuss approaches for writing (or storing) data in a charged polymer, e.g., DNA, using Add “0” and Add “1” enzymes and a deblock enzyme, as described therein.

The following commonly-owned U.S. patent application Nos. 63/369,339, and 63/369,340 contain subject matter related to that described herein, which is hereby incorporated by reference in its entirety to the fullest extent permitted by applicable law. The aforementioned commonly-owned patent application discusses other approaches for writing (or storing) data in a charged polymer, e.g., DNA, such as, using an AB Adapter instead of a deblock enzyme and using “A0B” and “A1B” for the Add “0” and Add “1” reagents, as described therein.

As discussed herein, the disclosure provides a novel system of storing (or writing or printing) information (or data) using a charged polymer, e.g., DNA, the monomers of which correspond to a machine-readable code, e.g., a binary (or other base) code, and which can be synthesized using a novel configuration of a piezo-electric inkjet printer system; novel methods and devices for synthesizing polymers in using a piezo-electric inkjet printer system, novel methods and devices for loading, writing, and unloading the polymers, and novel patterned silicon wafers for writing polymer on, which can be reliably fabricated, and method for fabricating same.

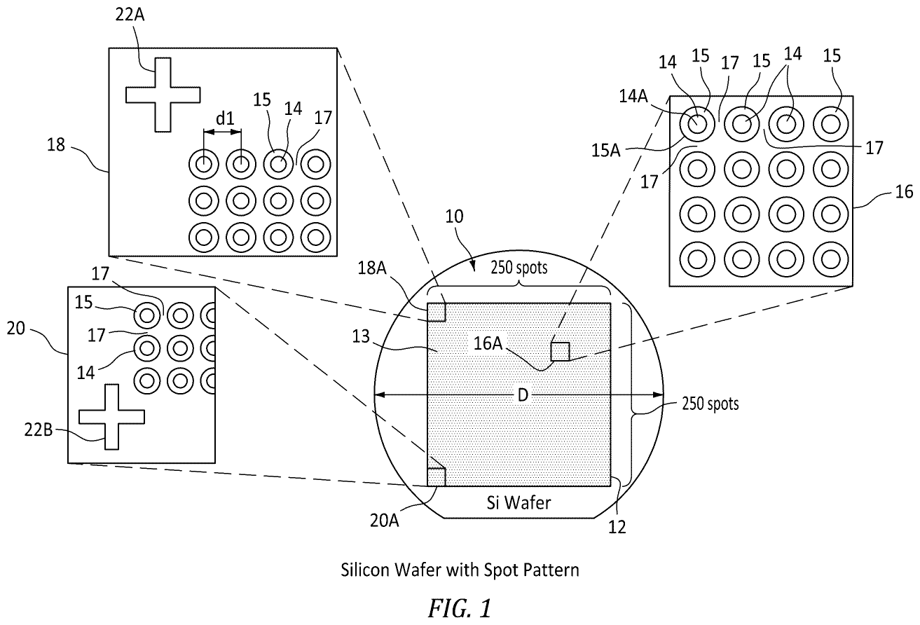

Referring to FIG. 1, a top view of a silicon wafer 10 with circular spot patterns 14 is shown, in accordance with embodiments of the present disclosure. In particular, the silicon wafer 10 has a square region or array or matrix 12 with a plurality of spots 14 (or printing spots or print spots or memory spots or reaction regions or reaction spots) on the upper surface of the wafer 10 that are designed to allow the successive writing of data on each spot 14 using a print head, e.g., a piezoelectric print head (discussed more hereinafter), while also minimizing chemical interaction between spots. Also, the size of the array 12 shown is a square having 250×250 spots, having dimensions of about 63.446 mm×63.446 mm, providing about 100 dpi (dots per inch) or a center to center spacing (or pitch) d1 of about 0.01 inches or 254 microns dispensed on the upper surface of a silicon wafer substrate 10. Other dimensions and number of spots and spot-spacing (or pitch) may be used if desired for the array or matrix 12.

The spots 14 are shown in three blown-up regions 16, 18, 20 from three regions 16A, 18A, 20A on the array 12 and may be shown as two concentric circles 14A, 15A, having an inner circle 14A and an outer circle 15A. The inner circle 14A is the outer surface of a raised circular spot pillar providing an active spot attachment area or region 14 surrounded by a circular separation or isolation channel or valley or depression region 15 having an inner surface 15A. The isolation channel 15 provides a physical barrier between adjacent spots 14 to avoid cross-contamination or chemical interaction between spots. In addition, one or more fiducial markers 22A, 22B may be provided or disposed on the wafer 10 for wafer alignment on the printer as shown on the upper left region 18 and lower left region 20 of the array area 12. Also, there may be interstitial areas or regions 17 between the spot pillars 14 and channels 15, which further isolates the spots 14 from each other.

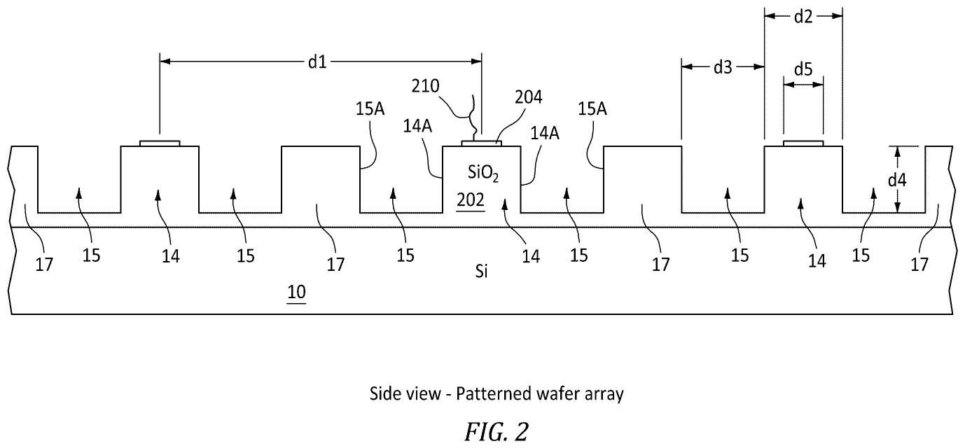

Referring to FIG. 2, a side view of a silicon wafer array of FIG. 1 with patterned layers for writing coded DNA on a surface is shown with dimension markings, in accordance with embodiments of the present disclosure. In particular, the silicon wafer 10 has a patterned layer 202 of SiO₂ on top of the wafer 10 to form the spot pillars 14 and channels 15. The distance dl (FIG. 1 and FIG. 2) is the center-to-center spacing between spots 14 also referred to herein as the pitch or spacing between spots or spot spacing. For a spot spacing d1 of 100 dpi (dots per inch), shown in FIG. 1, d1=about 0.01 inches or about 254 microns. Other dpi values or spot spacings may be used, such as 200 dpi, 300 dpi, 400 dpi, 500 dpi, 600 dpi, or higher, provided the spots do not chemically interact. Also, d2 is the width of the spot pillar 14, d3 is the width of the circular channel 15 around the pillar 14, and d4 is the height from the bottom of the circular channel 15 to the top of the pillar 14 walls, e.g., about 1 micron high. The distance d5 is the width (or length) of an attachment top coating 204, e.g., HfO₂, on top of the pillar 14 to enhance DNA starter strand attachment to the spot pillar 14. The width d5 of the HfO₂ coating may be slightly less than the width d2 of the top of the pillar 14. In some embodiments, the attachment top coating 204 width d5 may be substantially the same size as the pillar width d2. The HfO₂ attachment top coating 204 allows for easy stable attachment of DNA starter (or acceptor) strands 210 to the top of the pillar. Also, in some embodiments, the separation channel 15 may be treated or coated with a hydrophobic material (e.g. (fluorosilane or perfluoroalkyltriethoxysilane) to repel water which may be helpful during and after washing to prevent the washing fluid from chemically interacting between adjacent spots 14. Similarly, the top of the pillar 14 may be treated or coated with a hydrophilic material to attract water which may be helpful during and after washing to help prevent the washing fluid from chemically interacting between adjacent spots.

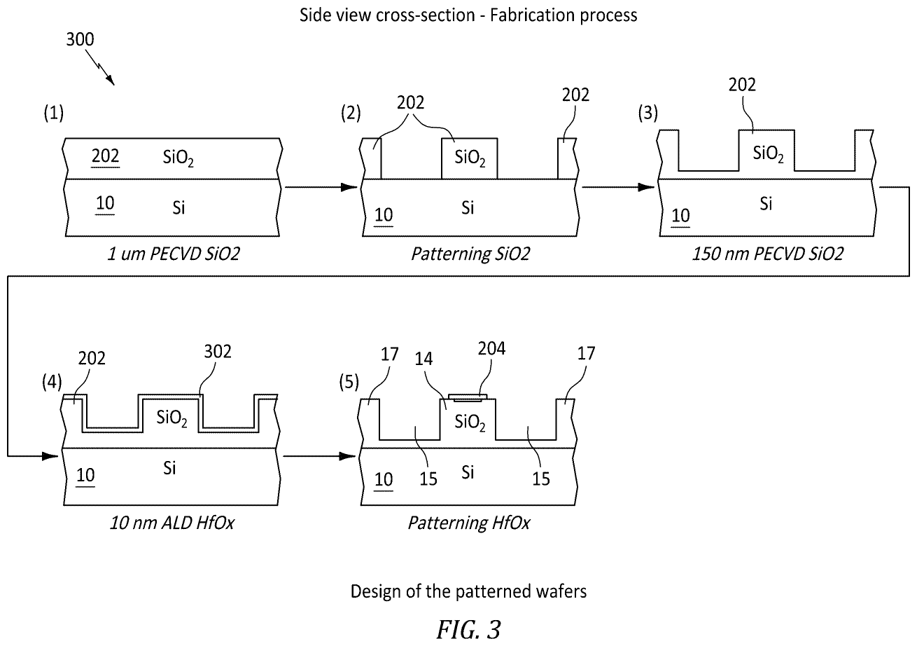

Referring to FIG. 3, a cross-section side view of a fabrication process 300 that may be used to create patterned wafers of FIGS. 1 and 2 is shown, in accordance with embodiments of the present disclosure. In particular, in Step 1, a 1 micron thick layer of silicon dioxide or silica (SiO₂) is applied or deposited onto the upper surface of the silicon (Si) wafer substrate using PECVD (Plasma-Enhanced, Chemical Vapor Deposition). Other techniques may be used to apply the SiO₂. Next, in Step 2, the SiO₂ is patterned or etched to create the desired pillars 14 and separation channels 15. Next, in Step 3, a 150 nanometer (nm) layer of SiO₂ is applied or deposited using PECVD onto the upper surface of the SiO₂ layer to coat the bottom of the channel. Next, in Step 4, a layer of metal oxide (hafnium oxide (HfO₂) is preferred, but other metal oxides such as titanium oxide or aluminum oxide could be used alternatively), e.g., ca. 10 nanometer (nm) thick, is applied or deposited to the upper surface of the SiO₂ layer, e.g. using atomic layer deposition (ALD). Next, in Step 5, the layer of the metal oxide, e.g., HfO₂, is removed or etched away except for at the top of the pillars 14 to create the DNA starter strand attachment region 204 on top of the pillars 14, leaving a circle of metal oxide, e.g., HfO₂ e.g., ca. 50-100 μm in diameter. Other approaches or processes may be used to create the same resultant shape and layer or region 204 if desired.

In some embodiments, a substrate, e.g., a silicon wafer or glass surface, undergoes thermal oxidation, e.g., to reach a target oxide layer of approximately 188 nm in thickness. This oxidized surface then undergoes photolithography, e.g., wherein a positive photoresist is spin coated onto the wafer surface, followed by exposure to the mask pattern forming the desired grid or substrate pattern, followed by development and rinsing of the surface. Next, a layer of metal oxide, e.g., HfO₂, is added to the patterned substrate surface, e.g., to reach a target metal oxide layer of approximately 70 nm in thickness. The photoresist mask on the substrate surface is subsequently lifted off of the underlying oxide layer, resulting in only a metal oxide layer atop an oxide layer on the substrate, with only the oxide layer surface between the patterned metal oxide spots. Following preparation of the wafer substrate, the metal oxide, e.g., HfO₂, is functionalized with a first surface modification, e.g., phosphonic acid, e.g., phosphonic acid linked to an azide-terminated alkyl linker. The interstitial oxide layer, e.g., SiO₂, between the metal oxide spots is functionalized with a second surface modifications, e.g., silane, e.g., silane with non-reactive or inert moieties to inhibit subsequent reactivity.

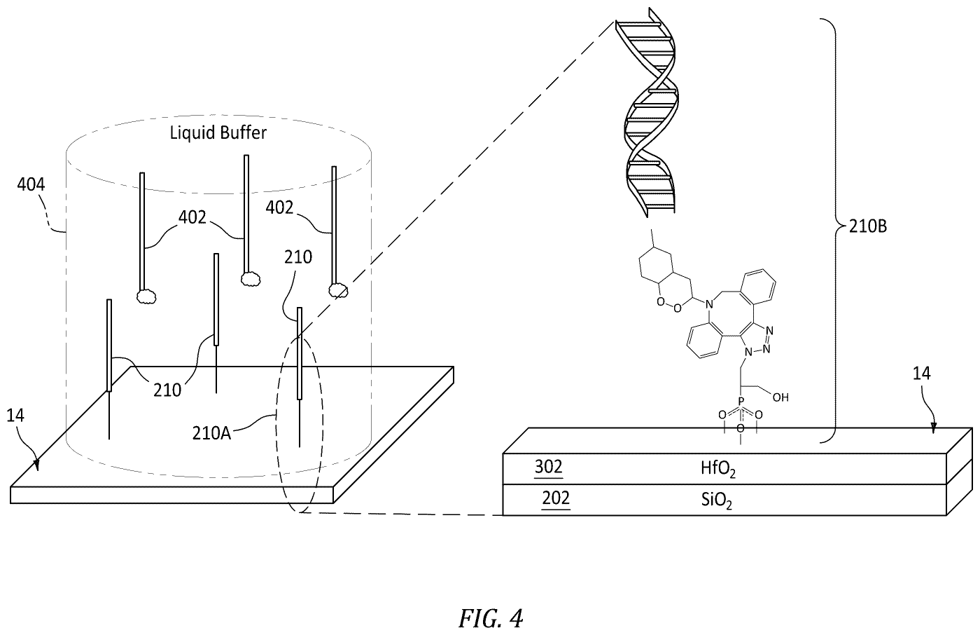

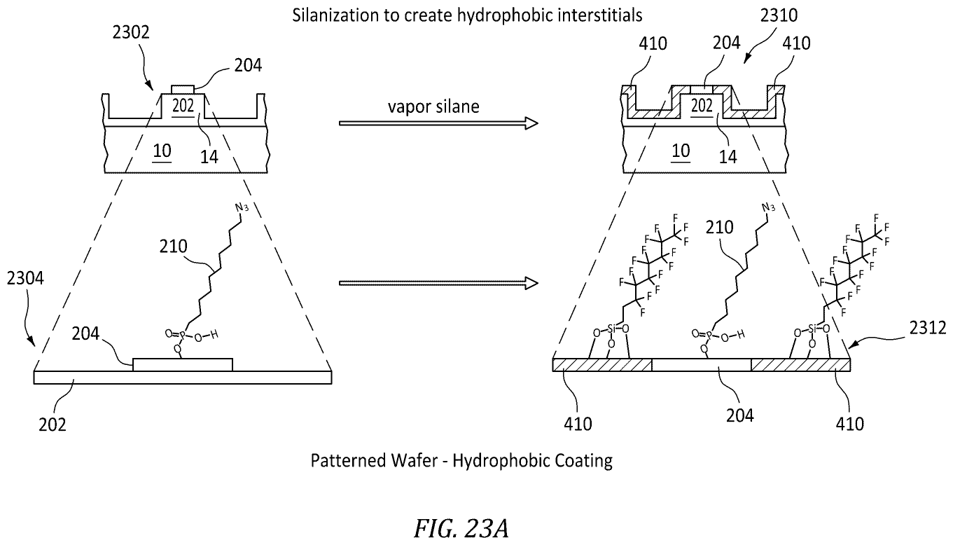

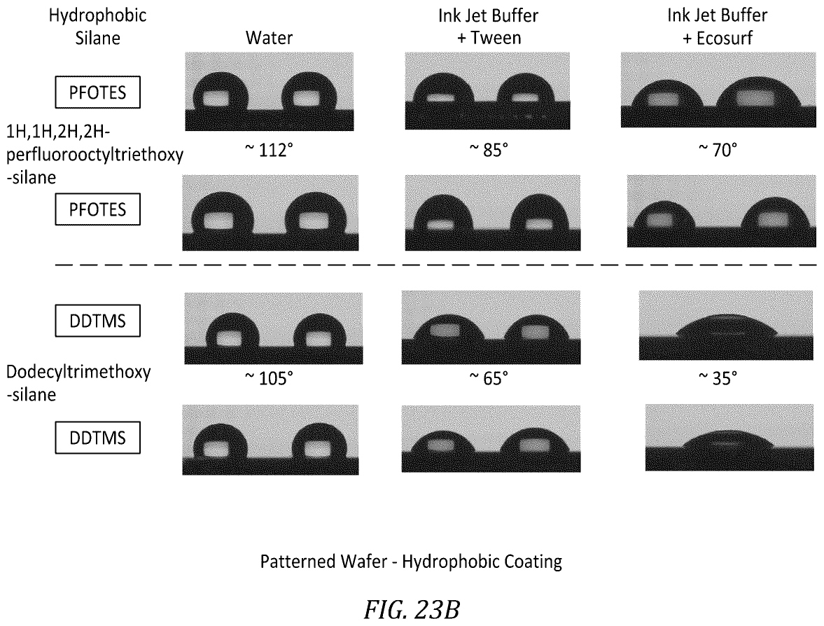

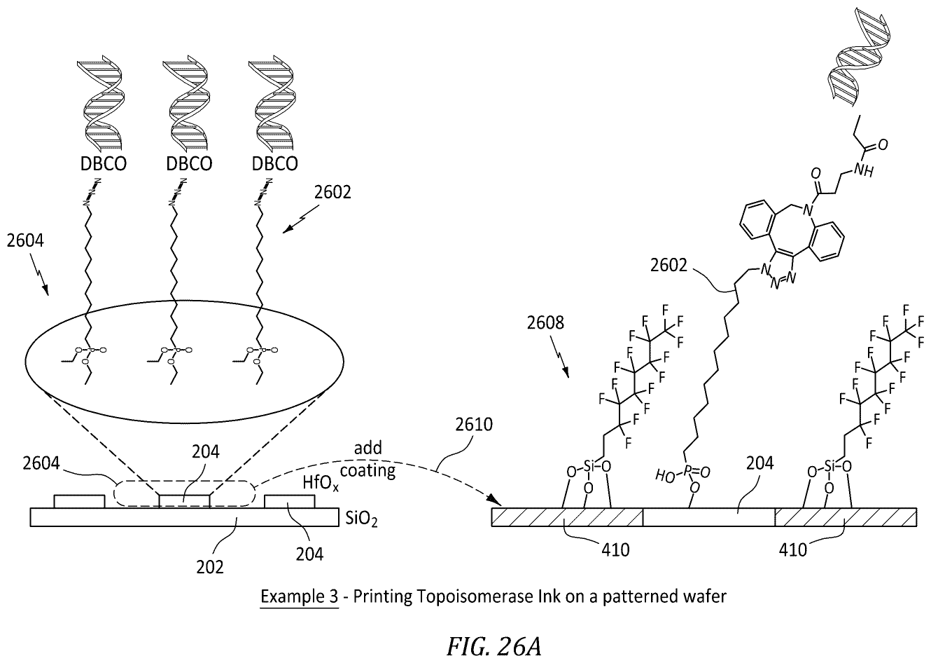

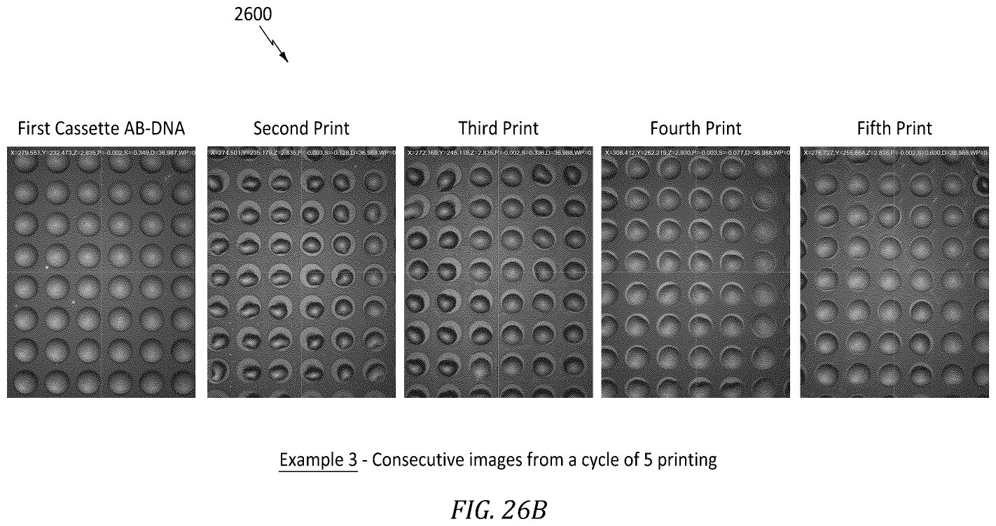

Referring to FIG. 4, a diagram showing an HfO₂ layer 302 for attachment of DNA molecules 210 to the wafer is shown and a blow-up of a portion of attachment region 210A with attached starter strand 210B, in accordance with embodiments of the present disclosure. In particular, a portion of the surface of the HfO₂ layer is shown on the pillar 14, with a plurality of starter strands 210 attached to the surface of the HfO₂ layer and data encoded DNA or polymers 402 suspended in a liquid buffer 404. The HfO₂ (Hafnium oxide) layer 302 is bonded to SiO₂ layer 202 on the substrate pillar 14 and used as a base layer to which starter or acceptor DNA is bonded. HfO₂ provides high dielectric constant (as an insulating material). The metal oxide surface (e.g., TiO₂, Al₂O₃, or HfO₂) surface can be functionalized, e.g., by selective phosphonation using a phosphonic acid linked to a reactive group (e.g., an azide moiety), via linker, e.g., a polyethylene glycol (e.g., PEG2-PEG6) or hydrocarbon linker, e.g., C₆₋₂₀ alkylene linker; for example, using azido-PEG3-phosphonic acid (available from BroadChem, catalog no. BP-23162), which comprises a PEG linker having an azide group at one end and a phosphonic acid moiety at the other, or 12-azidododecylphosphonic acid 95% from Sikemia, which comprises a dodecylene linker having an azide group at one end and a phosphonic acid moiety at the other. The phosphonic acid moiety will bind selectively to the metal oxide, e.g., HfO₂, rather than the SiO₂ or hydrophilic coating, while the reactive group can bind to a partner on the oligonucleotide. The reactive group which binds to the oligonucleotide can be, for example, a carboxy moiety which binds to an amine on the oligonucleotide, a streptavidin moiety which binds to a biotin moiety on the oligonucleotide, or a moiety capable of participating in a “click” chemistry reaction, such as an azide moiety which can bind to an alkyne-modified oligonucleotide via a “click” reaction. In certain embodiments, the click reaction is catalyst-free, for example a strain-promoted azide-alkyne cycloaddition (SPAAC), e.g., between the azide and a cyclooctyne, e.g., a dibenzocyclooctyne (DBCO) moiety or aza-dibenzocyclooctyne (ADIBO). moiety. See, e.g., FIG. 23A, depicting a HfO₂ surface functionalized by 12-azidododecylphosphonic acid and FIG. 26A, depicting an oligonucleotide linked to the 12-azidododecylphosphonic acid via a click reaction with an aza-dibenzocyclooctyne attached to the oligonucleotide. Once the starter or acceptor DNA strand 210 is attached or bonded to the surface of the pillar 14, e.g., to the HfO₂ attachment region 204, the free end of the strand 210 is available for attachment to a data encoded polymer or DNA strand 402, as discussed herein.

The surface bearing the oligonucleotides is optionally surrounded by a surface 410 bearing a hydrophobic coating, e.g., as depicted in FIGS. 23A and 26A. In these figures, the HfO₂ regions are deposited on a silica substrate, so the deposited HfO₂ surface bearing the oligonucleotides as described above is surrounded by the SiO₂ substrate regions, which are coated with a hydrophobic perfluorinated alkane substance, using 1H,1H,2H,2H-perfluorooctyltriethoxy silane, as seen in FIG. 23A, and/or the oligonucleotides are added to the HfO₂ regions using click chemistry as described, as seen in FIG. 26A.

Printing is not limited to a patterned silicon wafer but can be performed on a number of patterned or unpatterned substrates, e.g., silicon substrate, oxide surface, patterned hydrophilic/hydrophobic regions as defined by a depth difference (posts), hafnium oxide functional areas (as described above, with or without using posts), glass substrate, patterned hydrophilic/hydrophobic regions, polymer substrates, glass coatings, porous ceramic substrates, or ceramic coated paper.

For example, in certain embodiments, rather than using HfO₂ deposition on the silicon substrate, the silicon oxide surface may be silenized with a functionalized linker that contains DNA attachment moieties described above, e.g., for streptavidin-biotin or click conjugation.

A pattern of spots with DNA acceptor moieties can also be created using inkjet printing, for example by inkjet printing DNA in a desired pattern over a surface uniformly modified with phosphonic-acid and azide moiety.

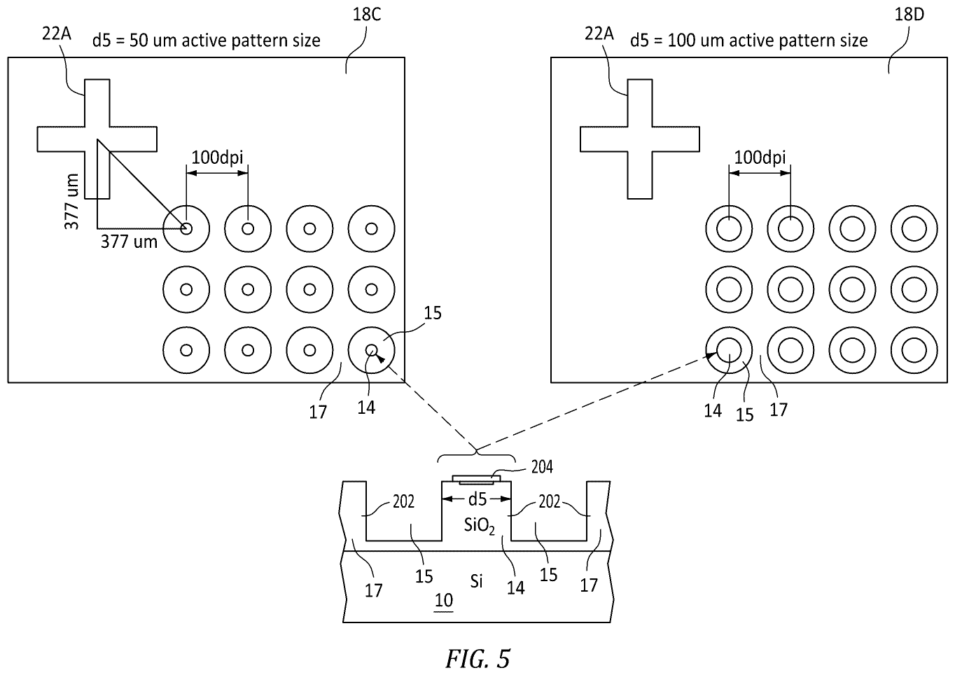

Referring to FIG. 5, a top view of a portion of two wafers 18C,18D with different spot pattern sizes on the spot pillars 14, and their relation to FIG. 2 is shown, in accordance with embodiments of the present disclosure. In particular, the upper left image 18C shows a 50 micron active pattern size d5 on top of the pillar 14 with 100 dpi and the upper right image 18D shows a 100 micron active pattern size d5 on the pillar 14 with 100 dpi.

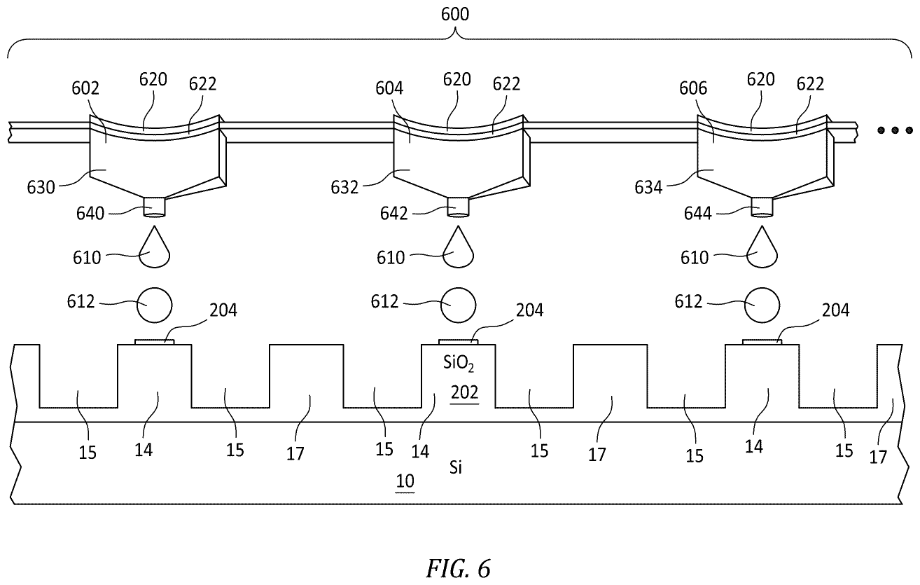

Referring to FIG. 6, a side view of a silicon wafer 10 with patterned layers 202, 204 having three nozzles 602, 604, 606 of a piezo-electric print head 600 and different stages of a released droplet 610, 612 is shown as the droplet 610, 612 travels toward the upper surface 204 of the pillars 14 on the patterned wafer, in accordance with embodiments of the present disclosure. As is known, a piezo-electric print head 600 uses a piezo-electric material 620 sandwiched with or attached to or disposed on a flexible plate 622, which flexes when a voltage is applied to the piezo-electric material 620, creating a diaphragm action as shown in FIG. 6. The flexing diaphragm 622 pushes out liquid 610 held in a chamber or print head 630, 632, 634 through a one or more nozzles 640, 642, 644 to provide precisely sized droplets 610, 612, which land on the tops of the pillars or reaction spots 204 of the array, discussed more hereinafter. In general, the design and control of such piezo-electric print heads 600 and inkjet delivery systems is well known by those skilled in the art, and may be found in the following issued U.S. Pat. Nos. 5,474,796; 6,921,636; 5,094,594; which are incorporated herein by reference to the extent needed to understand the present disclosure.

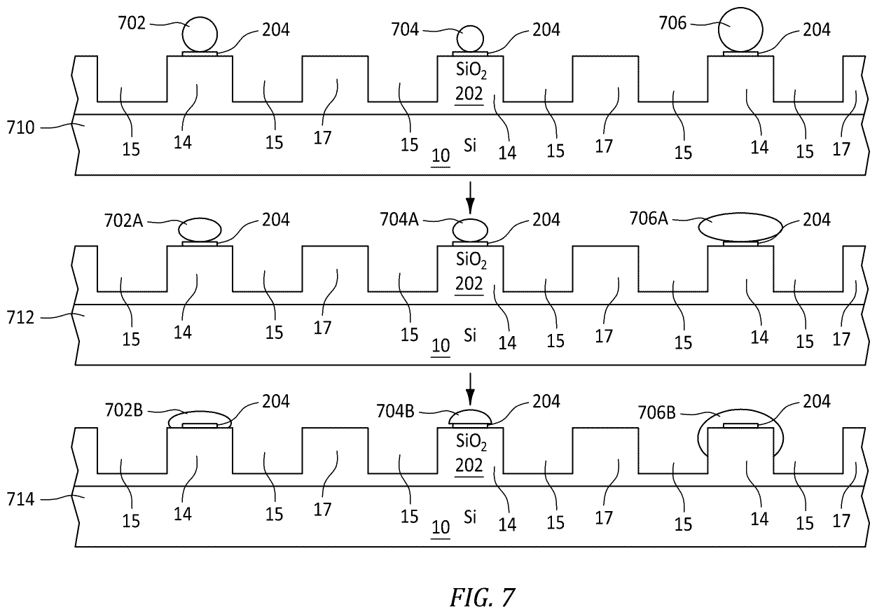

Referring to FIG. 7, a side view of a silicon wafer 10 with patterned layers 202, 204 is shown, including how the droplets change geometry after contacting the pillar 14 of a patterned silica surface, for three different size droplet 702, 704, 706 at three different times 710 (first contact), 712 (a time after first contact), 714 (final resting or steady state position of droplet), in accordance with embodiments of the present disclosure. In particular, the left most droplet 702, 702A, 702B is sized such that the final droplet position 702B stays on the pillar 14 but spills over the HfO₂ coating or attachment region 204 . The middle droplet (smallest) 704, 704A, 704B is sized such that the final droplet position 702B stays on the pillar and stays on the HfO₂ coating or attachment region 204. The right most droplet (largest) 706, 706A, 706B is sized such that the final droplet position 706B spills over the pillar 14 and into the separation channel 15.

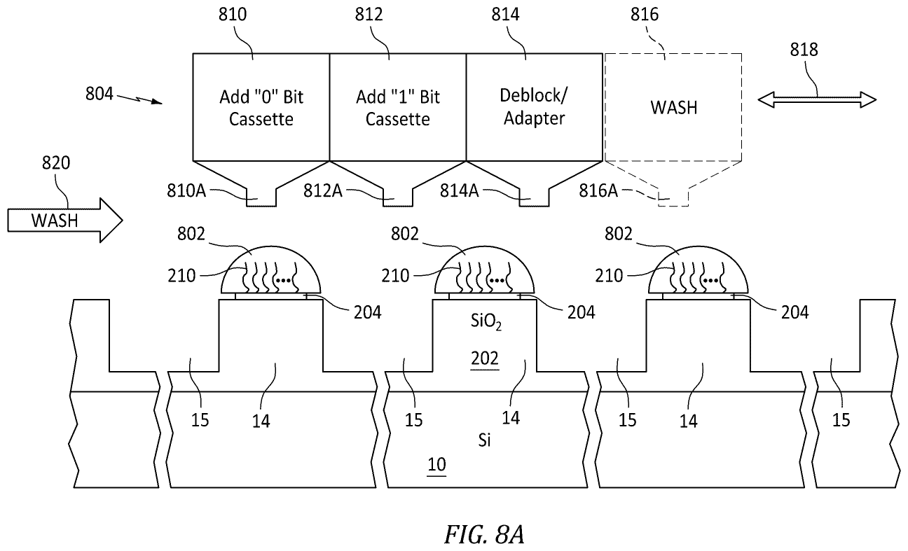

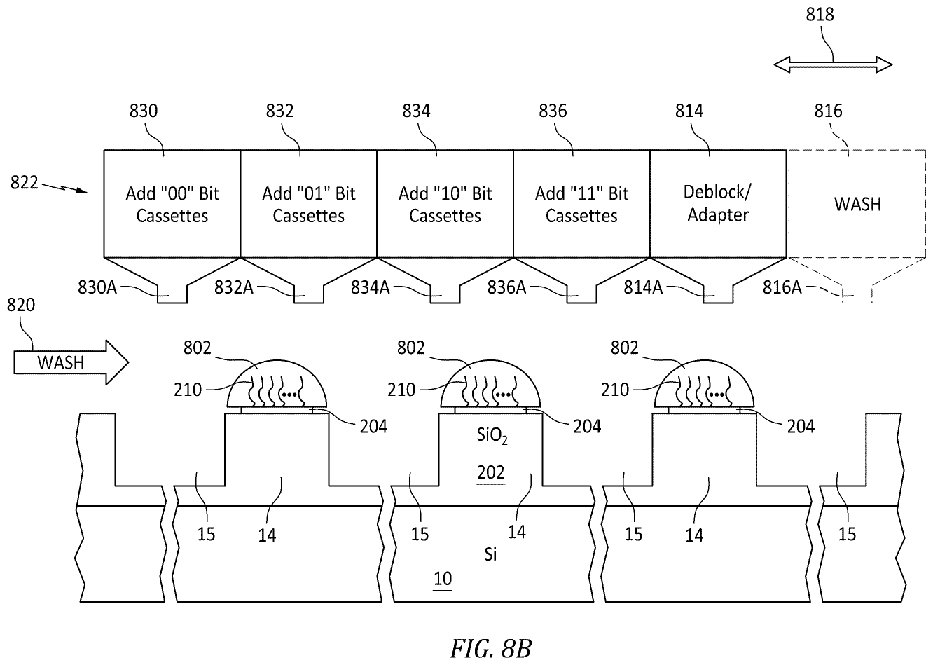

FIG. 8A shows a side view of a silicon wafer 10 with patterned layers 202, 204 showing starter DNA strands 210 in liquid 802 attached to pillars 14, showing a side view of a print head bank 804 having three or four print heads 810, 812, 814, with nozzles, and showing that a wash cycle using a wash fluid 820 may be spread or flowed or applied or sprayed horizontally across the wafer surface or applied vertically as a separate print head 816 as part of the print head bank 804, in accordance with embodiments of the present disclosure. The print head bank 804 may be controlled to move (as a group) as shown by arrows 818 across the wafer array to deliver the desired droplet at precise spot locations. In this case, the print head or print head bank 804 has three chambers 810, 812, 814, with associated nozzles 810A, 812A, 814A, respectively, with reagents used to add codes via droplets to the starter DNA strands 210 in the liquid bubble 802 shown on the top of each pillar 14, e.g., Add “0” head 810, Add “1” head 812, and Deblock/Adapter head 814. The Add “1” reagent may add a single base or a plurality of bases, which may be called a “cassette”, and the addition reaction chemistry functions the same as that described in the commonly owned US patents and patent applications.

FIG. 8B is similar to FIG. 8A, except that the print head bank 822 has four addition chambers or heads 830, 832, 834, 836, with associated nozzles 830A, 832A, 834A, 836A, respectively, which corresponds to 00, 01, 10, 11, respectively, for 2-bit binary encoding, for adding cassettes associated with same, in accordance with embodiments of the present disclosure. The print head bank 822 may include the deblock/adapter head 834, similar to FIG. 8A. Also, a wash cycle is shown using the wash fluid 820 (FIG. 8A) which may be flowed horizontally across the wafer surface or applied vertically as the separate print head 816 as part of the print head bank 822.

In some embodiments, the Add “0” and Add “1” chemistry used for writing to the polymer may be the chemistry described in the aforementioned commonly-owned US patents, where the center chamber would be a “deblock” chamber. Also, in some embodiments, the Add “0” and Add “1” chemistry used for writing to the polymer may be the chemistry described in the aforementioned commonly owned pending US patent applications where an “adapter” is used instead of a deblock enzyme and the Add “0” and Add “1” may be referred to as “A0B” and “A1B”, respectively. Accordingly, the action of getting the DNA strand ready to perform another addition reaction, may be referred to herein as a “deblock/adapter” or “adapter BA” action.

In some embodiments, the wash fluid 820 is flowed over the array 12 after an addition reaction to remove any unattached DNA strands and prepare the DNA for the next addition reaction or deblock reaction. In some embodiments, instead of or in addition to having the side flow wash shown, the print head bank 804, 822 may have an additional head chamber 816 with nozzle 816A that has a wash fluid in it that is dispensed during the wash cycles.

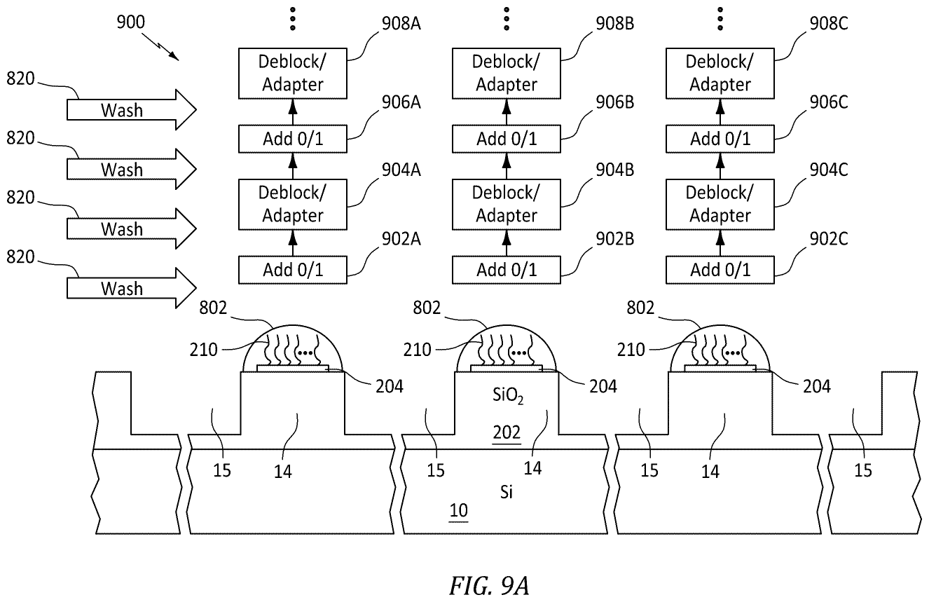

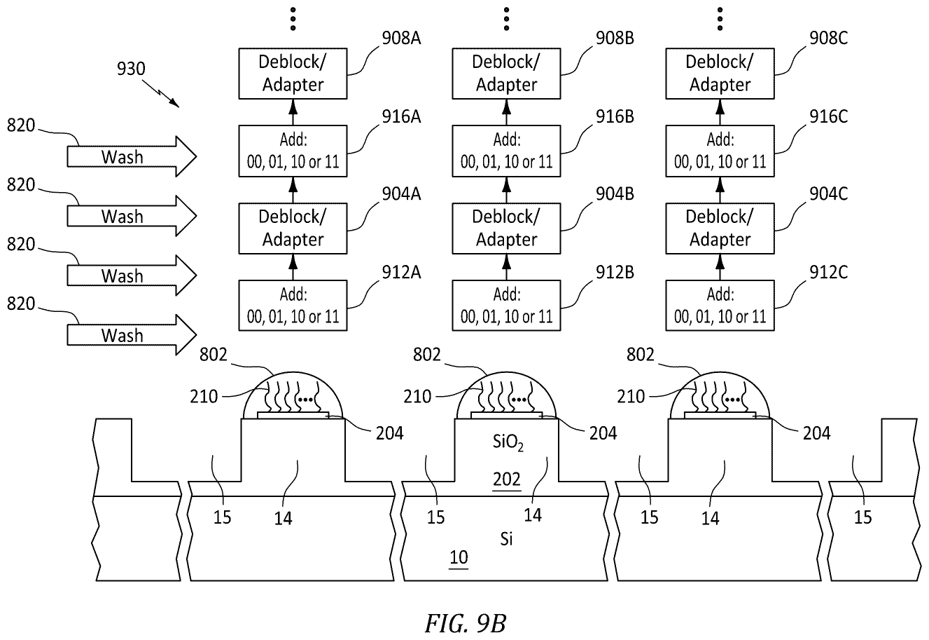

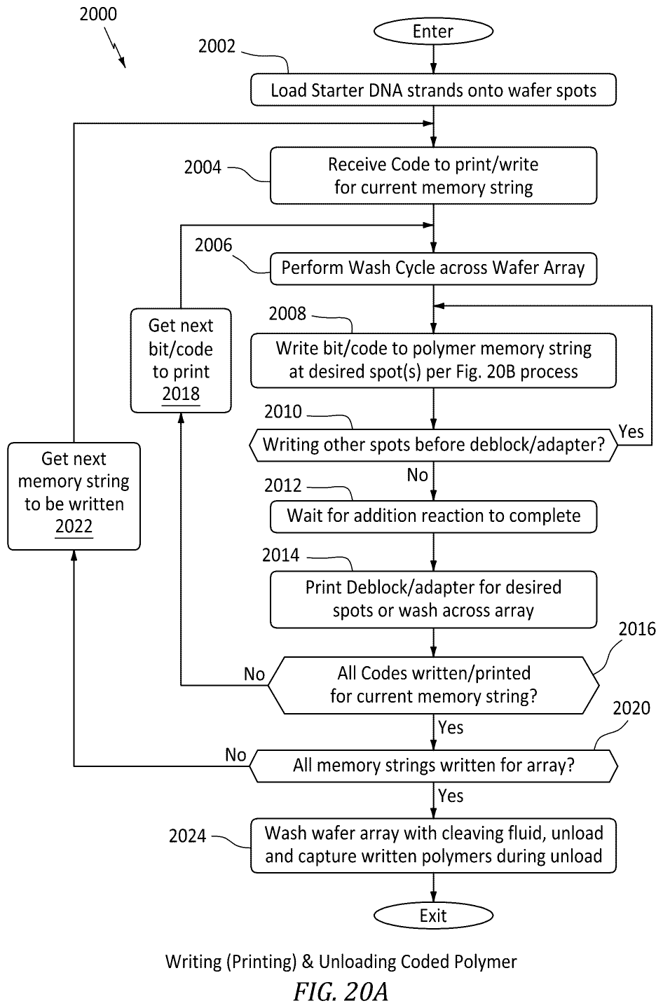

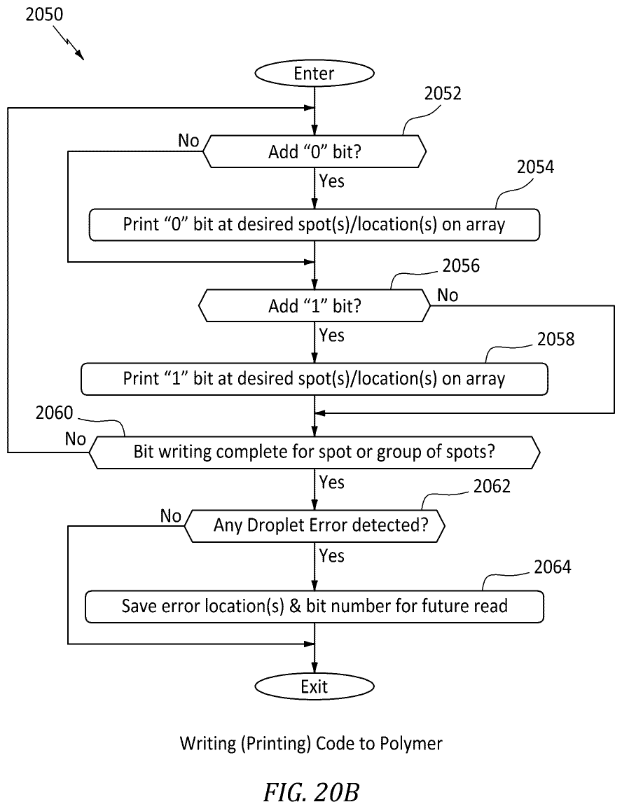

FIG. 9A shows a cross-section side view of a silicon wafer 10 with patterned layers 202, 204, showing starter polymer or DNA strands 210 in liquid 802 attached to pillars 14 and showing a side view of a data writing (or printing) process 900 to add bits or codes to the free end of starter polymer DNA strands 210 on the wafer, in accordance with embodiments of the present disclosure. In particular, a write addition begins by performing the wash cycle 820 to prepare the DNA strands 210 for the first write addition reaction. Next, the print head dispenses an Add “0” or Add “1” droplet onto the desired spot location(s), such as that shown in FIG. 6, depending on the desired bit or cassette to be written shown by blocks 902A, 902B, 902C. After the addition reaction is complete, a wash cycle 802 is performed to prepare the DNA strands for the deblock/adapter reaction. Next, the print head dispenses a Deblock/Adapter droplet onto the desired spot location(s) that have just had an addition reaction shown by blocks 904A, 904B, 904C. After the Deblock/Adapter reaction is complete, a wash cycle 802 is performed to prepare the DNA strands for the next addition reaction. Next, the print head dispenses an Add “0” or Add “1” droplet onto the desired spot location(s), depending on the desired bit or cassette to be written shown by blocks 906A, 906B, 906C. After the addition reaction is complete, a wash cycle 820 is performed to prepare the DNA strands for the deblock/adapter reaction. Next, the print head dispenses a Deblock/Adapter droplet onto the desired spot location(s) that have just had an addition reaction shown by blocks 908A, 908B, 908C. The above process repeats until all the desired bits or cassettes or codes have been written to the DNA strands. The write addition process is also discussed further with regard to FIGS. 20A and 20B hereinafter.

FIG. 9B shows a writing process 930 to add bits or codes to DNA strands similar to FIG. 9A, except that the write blocks 912A, 912B, 912C and 916A, 916B, 916C, are writing cassettes indicative of 2-bit binary codes 00, 01, 10, 11, using the print head bank 822 like that shown in FIG. 8B.

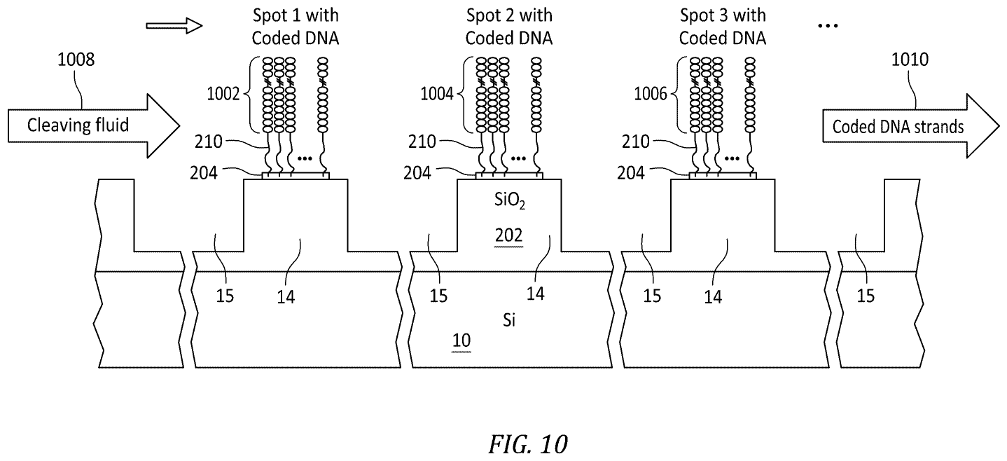

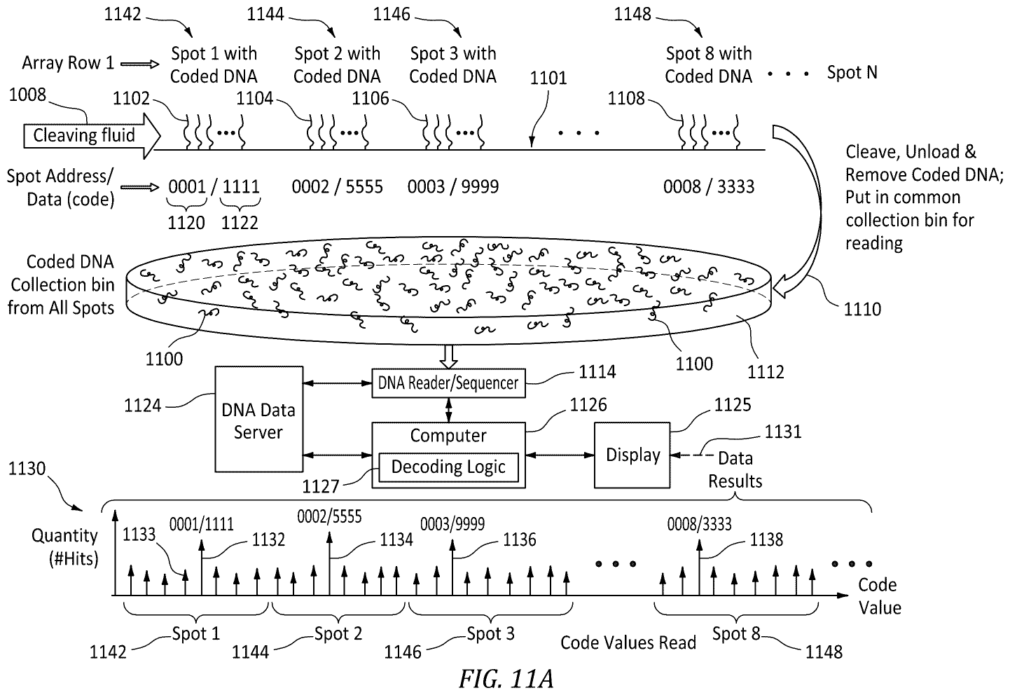

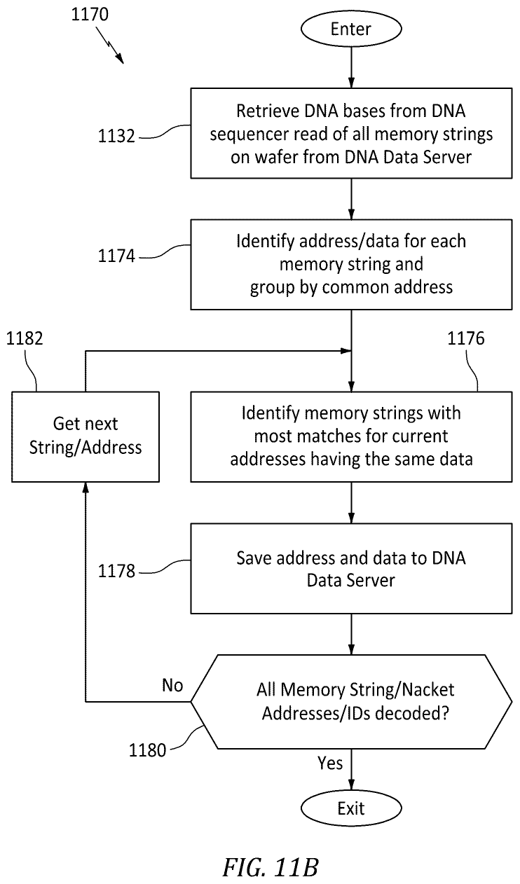

FIG. 10 shows a side view of a silicon wafer 10 with patterned layers 202, 204 showing starter DNA strands 210 attached to pillars at one end and attached to coded DNA on the other end, and also shows how a cleaving fluid 1008 may be used to remove the coded DNA strands 1002, 1004, 1006 from the wafer 10, in accordance with embodiments of the present disclosure. In particular, each pillar or spot 14 has a plurality of coded polymer or DNA strands 1002, 1004, 1006. When all the bits or cassettes or codes have been written or printed, a cleaving fluid 1008 may be flowed across the wafer array (or chip), which releases the coded DNA 1002, 1004, 1006 (which may include the starter strands 210) allowing them to be removed or flowed (shown by an arrow 1010) from the solid substrate 204 and placed in a storage container (FIG. 11A) which may contain liquid to keep the memory strings hydrated or may allow them to dehydrate for later re-hydration and reading.

Referring to FIG. 11A, a diagram showing an example of a plurality of spots 1142-1148, with coded DNA 1002-1008 (after writing codes) attached to a wafer shown as a flat surface 1101, and a process for removing, storing and reading the data written at each spot (Spot1-SpotN) is shown, in accordance with embodiments of the present invention. In particular, after the desired codes are written to the DNA memory strings (or strands or nackets) 1002-1008, each of the spots 1142-1148 having the coded DNA memory strings 1002-1008 attached can be unloaded and the coded DNA memory strings detached or removed from their respective spots (as discussed herein above). In some embodiments, there may be a plurality of coded DNA memory strings attached to a given spot (as discussed herein above). The detached coded DNA memory strings are then fluidically transported (shown by arrow 1110) along an output channel to a collection bin or container 1112 which holds the coded DNA strings from all the spots in a given wafer array outside of (or separate from) the wafer. When it is desired to read the stored data, the coded DNA memory strings, collectively 1100, in the collection bin 1112 may be read by any off-the-shelf DNA sequencer 1114 having an accuracy sufficient to meet the needs of the desired application, to determine the code written on each of DNA memory strings. The results of the DNA read of the code values may be analyzed by the decoding logic 1127 and a graph 1130 is shown to determine the codes with the highest quantity or hits.

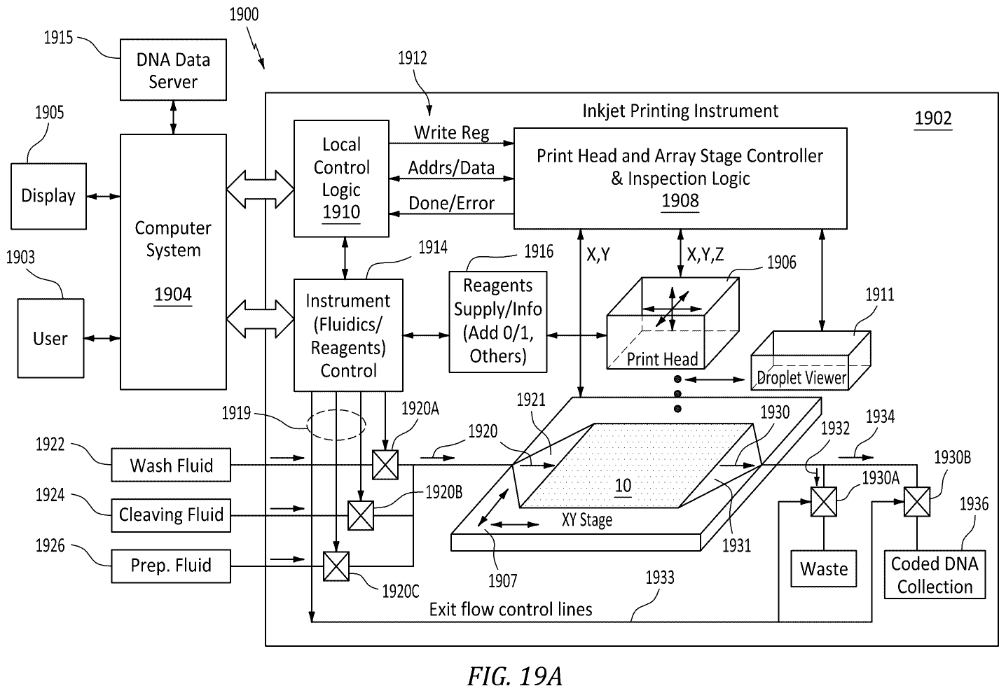

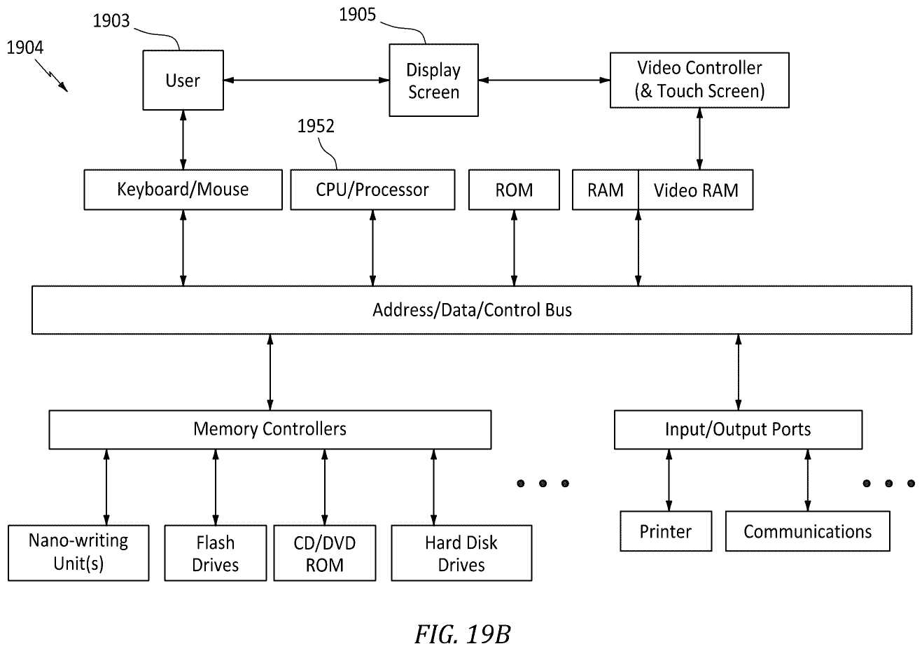

The DNA reader/sequencer 1114 may provide the code data values from the memory strings to a computer-based system 1126 which performs a decoding logic 1127 (discussed herein with FIG. 11B), which analyzes and decodes the data from the DNA sequencer 1114. The computer system 1126 may be such as that described herein in FIG. 19B or similar. The computer system 1126 may communicate with a DNA data server 1124 (similar to that of FIG. 19A) and may communicate with a display 1125, which may display or report data results 1130 from reading the DNA encoded data memory strings 1100. In some embodiments, the DNA Sequencer 1114 may save the code data directly to the DNA data server 1124, where it may be retrieved by the decoding logic 1127.

In some embodiments, the data may be written to the DNA string using a format of address/data 1120/1122, similar to that shown in FIGS. 11A or 11B, where the address or number of the spot 1120 being written to is coded, followed by the data 1122 associated with that address (or spot number), which may also include other error correction information and the like. In that case, the results may look like the graph 1130 shown on the bottom of FIG. 11A showing Quantity (#hits) vs Code Value for each of the coded DNA strings in the collection bin. In some embodiments, a plurality of spots may be written with the same data for redundancy and error correction purposes. In that case, the address may be used to identify all the spots or strands written with the same data. The resulting graph 1130 shows a distribution of values for each spot or address and the height of each line 1133 indicates the quantity of each code value read. It is expected that there will be some writing errors. Thus, each spot is populated with a plurality of DNA starter strings (as discussed herein) and they are all written simultaneously as described herein with a single drop from the printhead or prepopulated on the wafer. In some embodiments, the data associated with the address having the most number or quantity of the same value shown by tallest arrows 1132, 1134, 1136, 1138 for a given spot or address will determine the resulting values used for that spot or address/ID by the decoding logic 1127 (described herein below with FIG. 11B). The number of DNA strings or strands per spot will depend on the liquid spot size, e.g., about 10,000 to 1 million, and other quantities of DNA strings may be used if desired. Also, in some embodiments, for applications where the spot address is not important, e.g., if the coded DNA is left on the array the spot address may not be used or needed as part of the code.

FIG. 11B is a flow diagram 1170 for implementing decoding logic 1127 (FIG. 11A) for decoding polymer memory string data, in accordance with embodiments of the present disclosure. In particular, the logic begins at a block 1132 by retrieving from the DNA Data Server the DNA bases obtained from the DNA sequencer read of all memory strings on the wafer. Next, block 1174 identifies the address and data for each memory string and groups them by common address. Next block 1176 identifies the memory strings with the most matches for current addresses having the same data. Next, block 1178 save the address and data for the most matches to the DNA Data Server. Next, block 1180 determines whether all the memory string or nacket addresses or IDs have been decoded. If not, the logic proceeds to block 1182 to get the next string address and repeat the above process blocks 1176 to 1180. If the result of block 1180 is Yes, all memory strings have been evaluated and decoded and the logic exits.

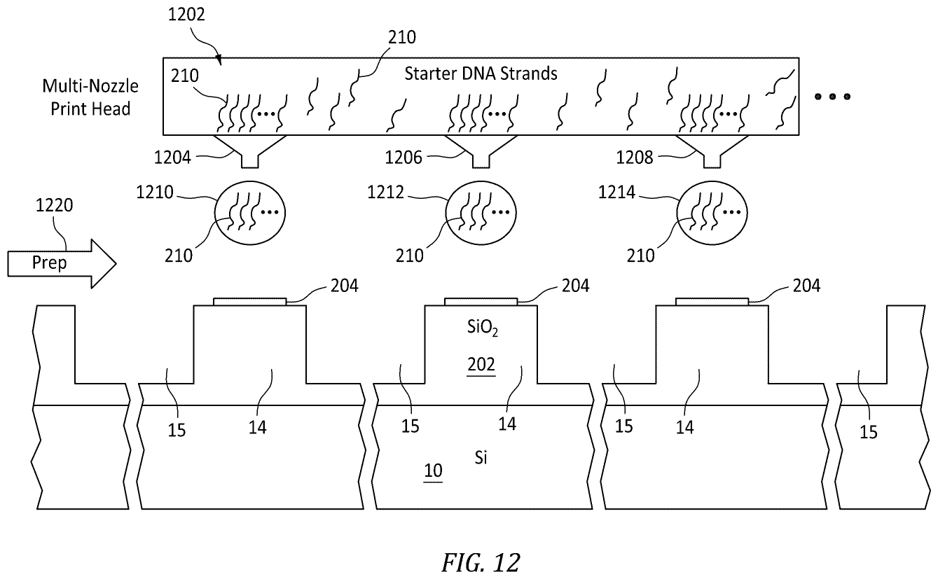

FIG. 12 shows a side view of a silicon wafer 10 with patterned layers 202, 204 showing a multi nozzle print head 1202 releasing droplets 1210 of starter DNA strands 210 onto the HfO₂ treated pillars 14 (or spots) of the wafer array, and showing an optional preparation fluid 1220 washed across the surface to prepare the wafer for starter DNA attachment, in accordance with embodiments of the present disclosure. In that case, depending on the number of nozzles 1204-1208, the DNA may be able to be loaded all simultaneously, or a group at a time.

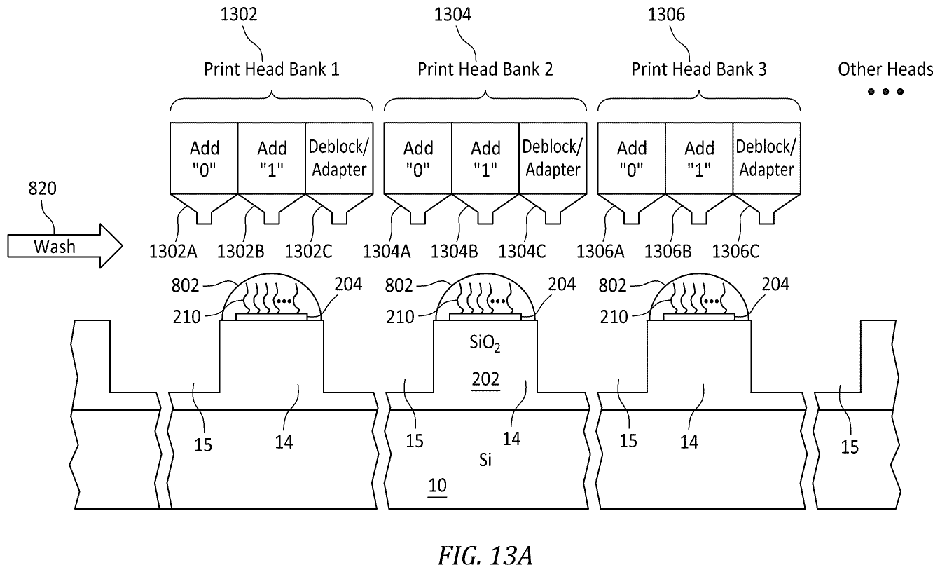

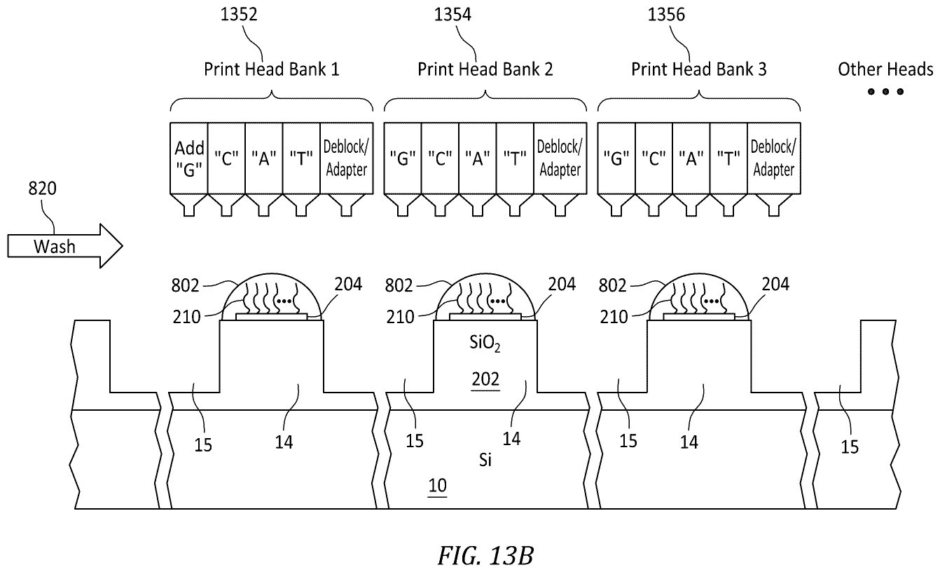

FIG. 13A is the diagram of FIG. 8A for Add “0”, Add “1”, and Deblock/Adapter, where the three nozzles are grouped together to form a bank and a plurality or array of print head banks 1302, 1304, 1306 are used to write or print to a plurality of corresponding spots 14 on the wafer array. In that case, in the array of print head banks, each bank 1302, 1304, 1306 having three nozzles, e.g., 1302A, 1302B, 1302C for print head bank 1302; 1304A, 1302B, 1302C for print head bank 1304; and 1306A, 1306B, 1306C for print head bank 1306 for printing or writing data to a plurality of spots simultaneously, would greatly speed up the writing or printing process.

FIG. 13B is the diagram of FIG. 13A where the three nozzle print head banks are replaced by five nozzle print head banks, e.g., G, C, A, T, plus deblock/adapter, for printing to a plurality of spots simultaneously, in accordance with embodiments of the present disclosure. In that case, the number of different types of bits is base four (G, C, A, T), which expands the amount of data that can be stored. As discussed herein, each of the bits may be a single base, or may be a plurality of bases (or a cassette). In the case of a cassette, the four bases listed, would merely be a label indicative of the four possible states for each digit or position or bit in a word, depending on the type of encoding used, e.g., for 2-bit binary encoding they would represent 00, 01, 10, 11, cassettes (or strings of bases), as shown in FIG. 8B.

In particular, DNA using four bits (or bases or groups of bases) representing GCAT data to be written, using any number of “bits” (or monomers or bases) may be used if desired for the data storage polymer (or memory string), provided they meet the desired functional and performance requirements. More specifically, referring to FIG. 13B, a side view of 4 addition print head banks 1352, 1354, 1356 are shown, each of the four add heads or nozzles or chambers has a unique chemical construct (or monomer or plurality of bases or cassette) or code that is added to the polymer memory string. For a base-4 system (e.g., GCAT, for DNA based system), there would be 4 add heads or chambers or nozzles and a single deblock/adapter head or chamber or nozzle (5 total). This can be viewed as four (4) unique codes, which in binary would be 00, 01, 10, 11 (or 0 to 3 in decimal). The four codes could also be the four bases in DNA, i.e., GCAT, as discussed herein and in the aforementioned commonly-owned patents and patent applications. Such a configuration enables the bulk writing of information or data (multiple bit writing) with a single (multi-nozzle) print head bank, which increases the storage density of data and speed at which the data can be stored, over writing a single bit during each write cycle (or add reaction). This can be done for any number of unique addition print heads or chambers or nozzles that provide a unique code (or chemical item or construct) for a given spot, the only limit is the number of unique chemical items or constructs (or cassettes) that can be added or written to the memory string (or polymer), and that can be identified (or read), as described herein.

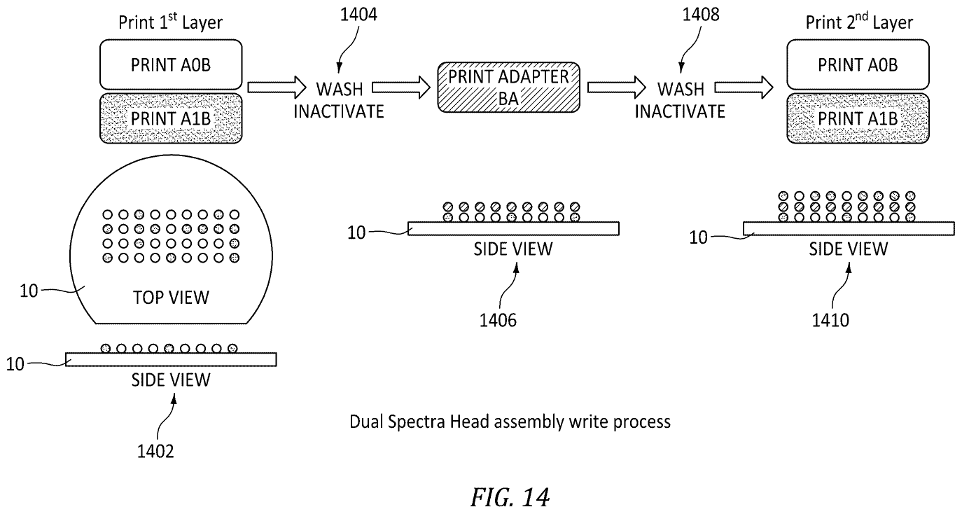

FIG. 14 shows a diagram of a dual print head assembly writing process, showing an entire row and layer of 1's and 0's being written followed by an adapter layer being written, followed by another layer of 1's and 0's, in accordance with embodiments of the present disclosure. In that case, such a dual head assembly (or dual nozzle or dual chamber) allows the print head to write an entire layer of 1's and 0's (or “1” cassettes and “0” cassettes) before having to deblock the layer to enable the next layer to be written. In particular, on the left side at 1402, the A0B and A1B for the first layer are printed (using 2 heads), which shows a top view and side view of the first layers of printing “0”s and “1”s. Next, a wash step 1404 is performed, to wash the surface with a buffer to inactivate or wash off all unbound molecules. Next, at 1406, the Adapter BA is printed and a corresponding side view is shown below. Next, a wash step 1408 is performed again as before. Next, at 1410, the second layer of printing “0”s and “1”s again using A0B and A1B is performed, and a corresponding side view is shown below. Such a dual head assembly can print two cassettes at the same time over different reaction spots, which allows for faster data writing, small volumes, so minimal material loss, and may require buffer optimization for efficiencies of cycle conversions in small volumes and control evaporation of spots liquid.

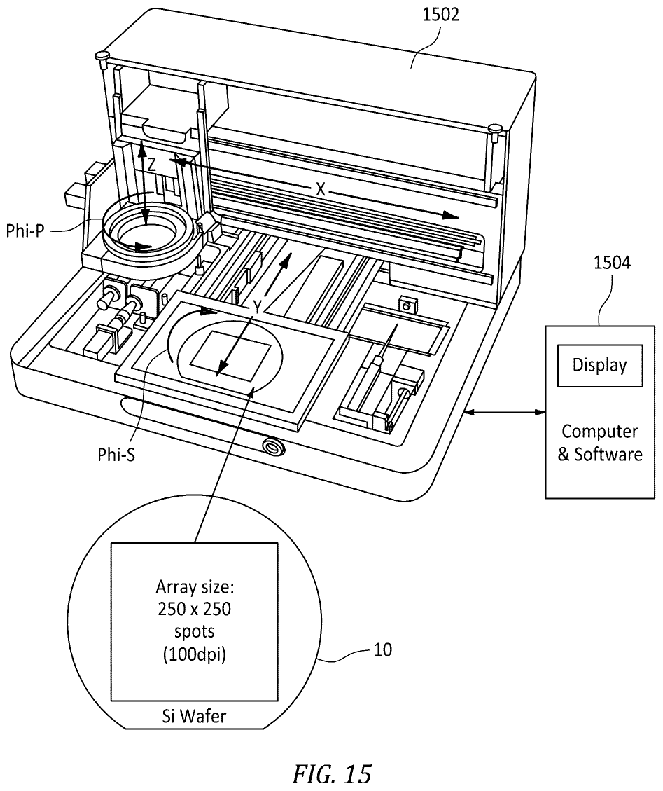

Referring to FIG. 15, an image of an inkjet printer 1502 set up showing motion axes and a computer controller 1504 and a zoom-in of a wafer 10 is shown, in accordance with embodiments of the present disclosure. In some embodiments, the printed section of the wafer may be about a third of the wafer; other portions of the wafer may be used for printing if desired. The printer used was an LP50 inkjet printer, made by SUSS MicroTec, using PiXDRO technology.

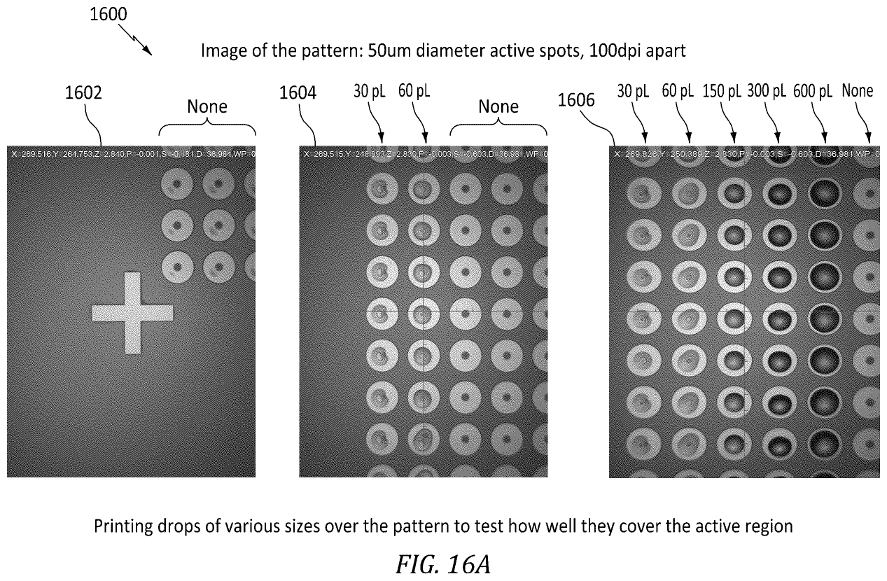

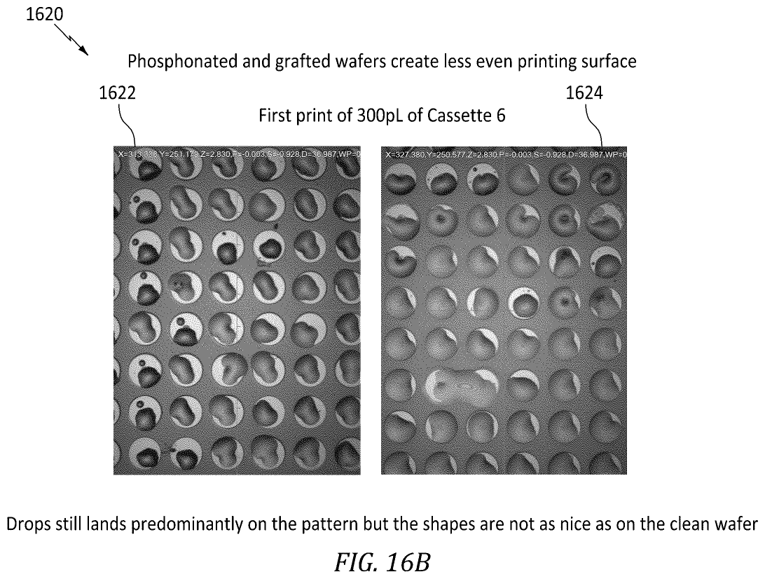

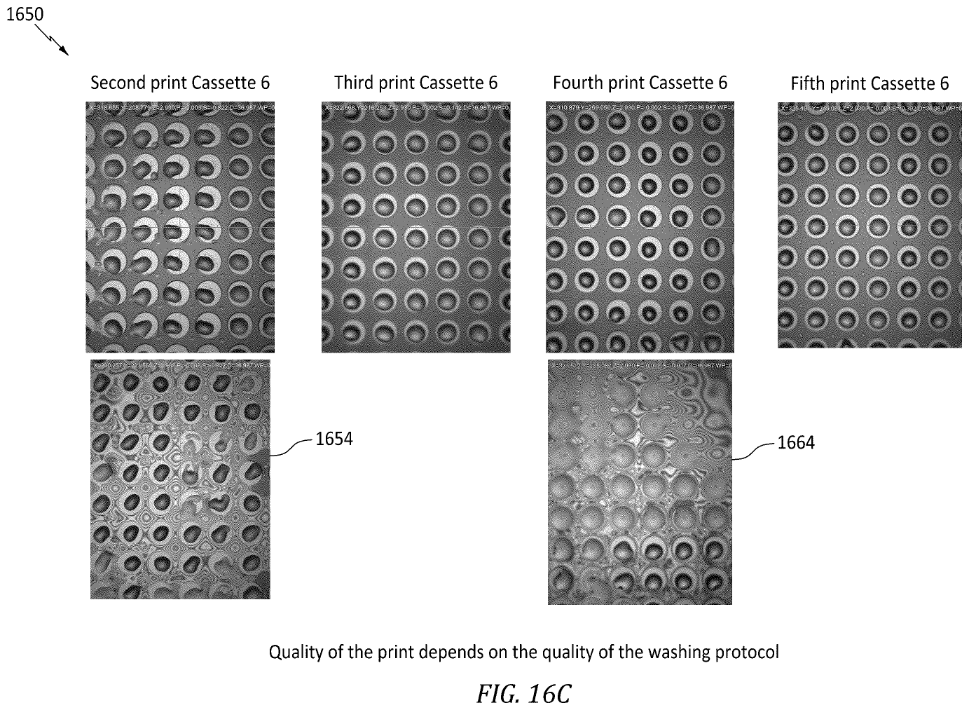

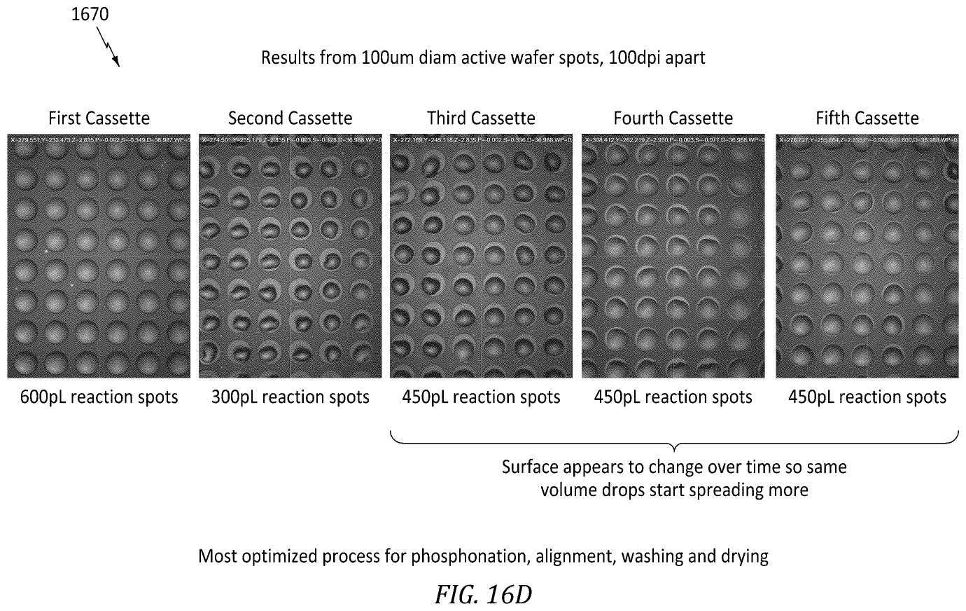

FIG. 16A shows images 1600 of printing drops of various sizes for 50 micron diameter active spots and how they cover the active region or pillar are shown, in accordance with embodiments of the present disclosure. Image 1602 shows active regions with no drops. Image 1604 show active regions with columns for 30 pL, 60 pL, and None spots left to right. Image 1606 shows regions with columns of 30 pL, 60 pL, 150 pL, 300 pL, 600 pL and None left to right.

Referring to FIG. 16B and FIG. 16C, images of results of printing and washing five times for 50 um diameter active wafer spots, 100 dpi apart using 300 pL are shown, in accordance with embodiments of the present disclosure. The topogation protocol, including printing number of layers and drop size (e.g., print cassette in 10% PEG, 5% Glycerol, 0.1% Tween—10 layers of 30 pL drops) and wash details (e.g., 4× wash with 1M NaCl/0.05% Tween Wash buffer; Rinse in Topogation Buffer, Deposit cassette 5 by hand; 4× wash 1M NaCl/0.05% Tween Wash buffer; Wash Topo Buffer (No PEG, Glycerol or Tween); Try drying out with or without air—patterned wafers hold onto liquid and are harder to dry. It also shows spot merging in certain areas, which would be reduced or eliminated with the use of hydrophobic treatment outside the pillars. FIG. 16B shows first print and FIG. 16C shows prints 2 through 5. Images 1654 and 1664 show the wet section of the wafer for the second and fourth prints showing significant spot liquid overlap from washing and not complete drying.

FIG. 16D shows images 1670 of results of printing and washing five times for 50 um diameter active wafer spots, 100 dpi apart, are shown, in accordance with embodiments of the present disclosure. The reaction spots sizes ranged from 300 pL to 450 pL to 600 pL.

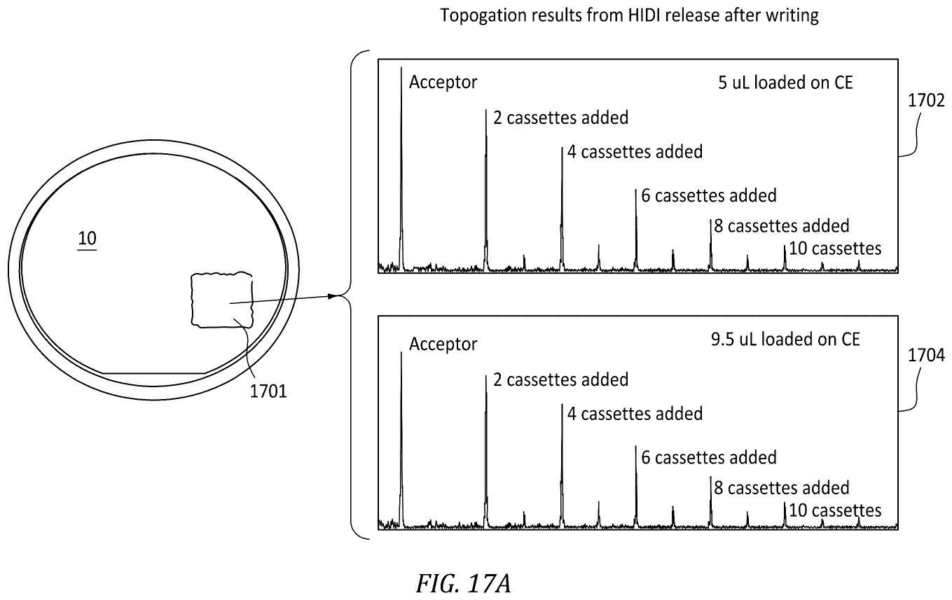

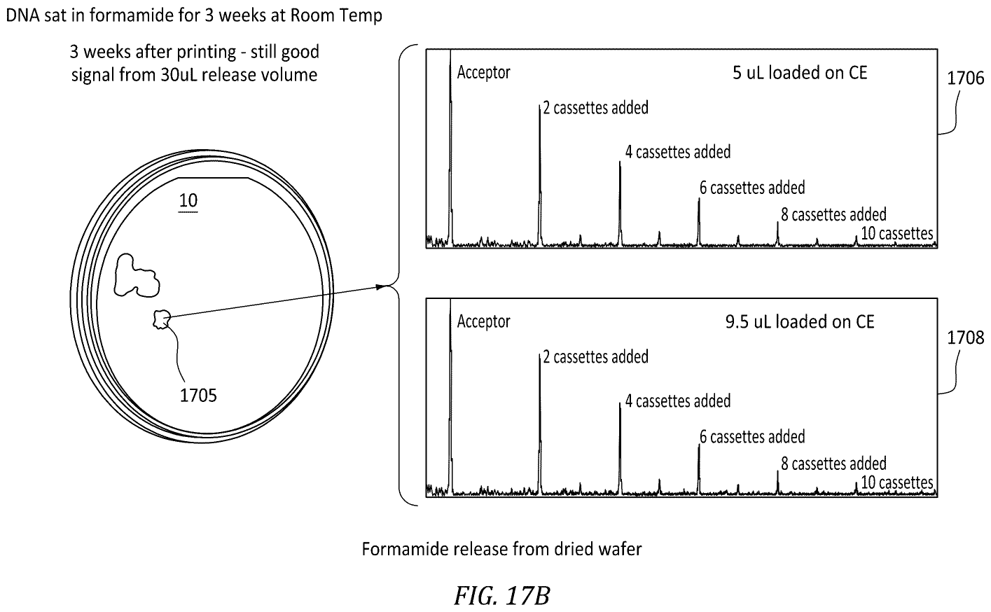

FIG. 17A shows an image of the wafer 10 and two graphs 1702, 1704 showing mass spec topogation results from HIDI release after writing from a portion 1701 of the wafer, in accordance with embodiments of the present disclosure. In particular, graph 1702 shows data from a 5 microliter loaded on CE, and graph 1704 shows data from a 9.5 microliter loaded on CE. This shows that the 10 bits or cassettes were able to be written and read back.

FIG. 17B shows an image of the wafer 10 after drying out for three weeks and two graphs 1722, 1724 showing mass spectrometry results from formamide release from a portion 1705 of a dried wafer 10, in accordance with embodiments of the present disclosure. This shows that the data is preserved after dehydrated storage 3 weeks after printing and good signal was received from 30 microliters (uL) release volume. The DNA sat in formamide for 3 weeks at room temperature. In particular, graph 1706 shows data from a 5 microliter loaded on CE, and graph 1708 shows data from a 9.5 microliter loaded on CE.

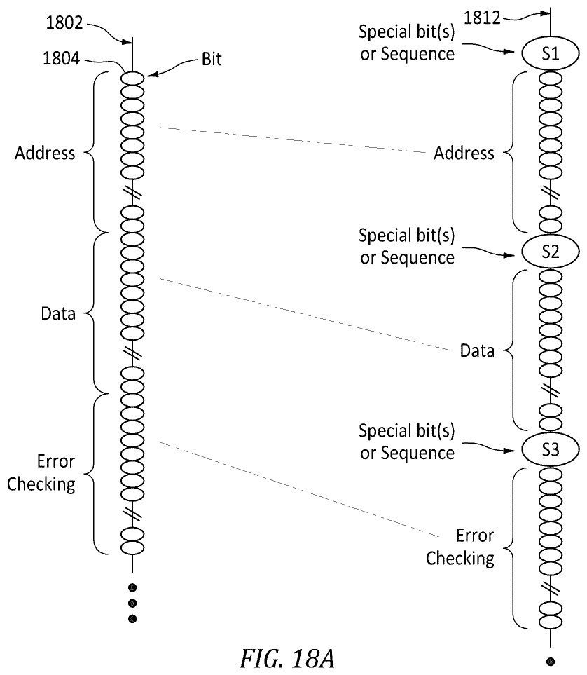

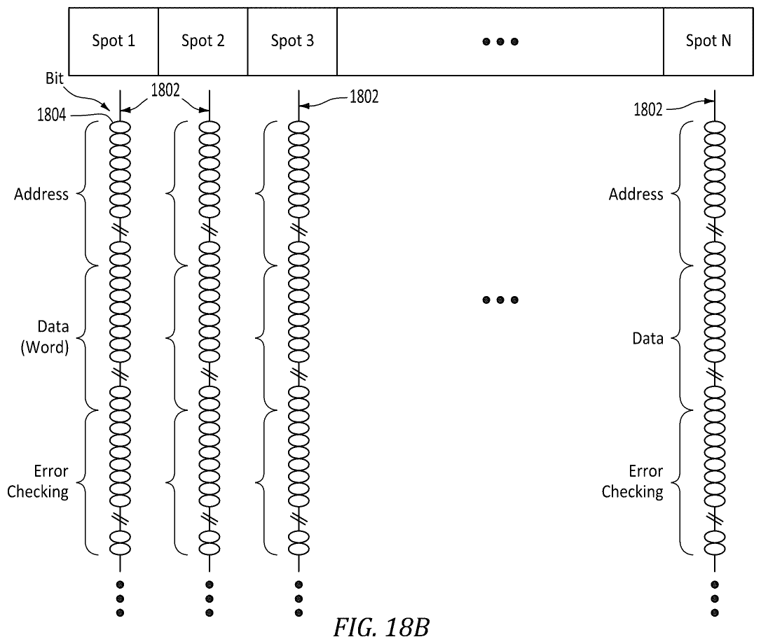

Referring to FIGS. 18A and 18B, the format of how data written to the polymer may vary based on various factors and design criteria. In particular, the “memory string” (or memory strand or DNA or polymer) 1802 may be shown as a line on which are a series of ovals 1804, indicative of individual “bits” written (or added) which may be in the form of a cassette (or string of DNA bases) onto the memory string in a given memory cell. In some embodiments, the bits 1802 may be written one after the other to build a “storage word”. A first example data format shows three components to the storage word, an address section, a data section, and an error checking section. The address section may be a label or pointer used by the memory system to locate the desired data. Unlike traditional semiconductor memory storage where hardware address lines on a computer memory bus would address a unique memory location on the physical memory chip, the nano-writing system of the present disclosure may have the address (or label) be part of the data stored and indicative of where the data desired to be retrieved is located. In the examples shown in FIGS. 18A and 18B, the address for the data written to each spot or plurality of spots for redundancy is located proximate to or contiguous with the data, as well as error checking data, such as parity, checksum, error correction code (ECC), cyclic redundancy check (CRC), or any other form of error checking and/or security information, including encryption information. In the storage word, each of the components Address, Data, Error Checking, are located after each other in the memory string 1802. As each of the components have a known length (number of bits), e.g., address=32 bits, data=16 bits, error check=8 bits, each storage word and its components can be determined by counting the number of bits. Also, as discussed in the aforementioned commonly owned issued patent and patent applications, a given bit 1804 may be represented by one or more DNA bases or oligomers or the like. When a plurality of bases are used to represent a bit (i.e., a “0” or “1” for a binary system, or G, C, A, T, for a base 4 system), they may be referred to as a “cassette”, as discussed herein. Thus, as used herein, the term bit and cassette may be used interchangeably. In some embodiments, there may be a plurality of digital words (address, data, error checking) stored on a given DNA memory string, depending on how long the DNA string can be written.

Referring to FIG. 18A, the right side is an example data format with the same three components as memory string 1802, address section, data section, and error checking section. However, for memory string 1812, in between each of the sections there is a “special bit(s) or sequence” sections S1, S2, S3. These special bits S1, S2, S3 may be a predetermined series of bits or code that indicate what section is coming next, e.g., 1001001001 may indicate the address is coming next, whereas 10101010 may indicate the data is coming next, and 1100110011 may indicate the error checking section in next. In some embodiments, the special bits may be a different molecular bit or bit structure attached to the string, such as dumbbell, flower, or other “large” molecular structure that is easily definable when the DNA memory string is read offline, outside of the nano-writing chip described herein. Instead of it being large, it may have other molecular properties that provide a unique change to the polymer construction for the 1 bits and 0 bits, as discussed herein above.

FIG. 19A is a block diagram showing an inkjet printing system 1900 including an inkjet printing instrument 1902 and a computer system 1904 which interfaces with the instrument 1902. The inkjet printing instrument 1902 may include the piezo-electric inkjet print heads 1906 (similar to those discussed herein), which deliver the reagent droplets discussed herein to the desired writing spots on the wafer array 10, which is mounted to an XY stage 1907. The print head and XY stage may be controlled by a print head and array stage controller and inspection logic 1908 which communicates with Local Control Logic 1910 to write the desired reagents and codes to the DNA strands as directed as discussed herein. For example, one or more of the read/write address and/or data inputs, outputs and/or control lines, may be received from or provided to a serial (or parallel) bus, which includes digital commands for which codes or data to write to the array. The Computer System 1909 may receive commands from a user 1903 and provide information to a display 1905 for use by the user 1903, and may also provide commands to the local control logic 1910 which provides specific write requests to the print head 1106 and array stage controller and inspection logic 1908. The print head 1906 and array stage controller and inspection logic 1908 controls the print head position XYZ and the wafer array XY stage 1907, and also receives data from a droplet viewer (or sensor) 1911 to determine the quality of the drops and reports results and errors back to the local control logic 1910 and the computer system 1904 which may store the droplet error information on a DNA Data Server 1915 or other memory device for future use when reading the data. Such information may be used to correct or ignore certain data that is known to have certain errors in the data caused by droplet errors.

The inkjet printing instrument 1902 may include instrument (fluidics/reagents) control logic 1914 which controls the reagent supplies 1916 to the print head and controls the fluid flows 190 through a flow inlet manifold 1921, across the wafer array 10, e.g., wash fluid 1922, cleaving fluid 1924, preparation fluid 1926, and the like, via valves 1920A, 1920B, 1920C, respectively, and control lines 1919, as well as controls the exiting fluids 1930 which flows through a flow exit manifold 1931, such as the waste fluid 1932 via valve 1930A and control lines 1933, and the fluid 1934 having the coded DNA that has been detached from the wafer array, via valve 1930B and control lines 1933, and collected, e.g., in a collection bin 1936, for later reading.

FIG. 19B is a block diagram of the computer system 1904 of FIG. 19A, in accordance with embodiments of the present disclosure. The Computer System (FIG. 19B) 1904 may interact with the inkjet printing instrument 1902, and may also interact with the instrument control 914 which interacts with separate fluid supplies and the like, all of which interact with one or more CPU/Processors 1952, or logic for performing certain functions described herein. Also, the Computer System in FIGS. 19A and 19B may interface with a user 1903 and a display screen 1905.

The Local Control Logic 1910 (FIG. 19A) and the Fluidics Instrument Control 1914 and the print head and array stage controller 1908, have the necessary electronics, computer processing power, interfaces, memory, hardware, software, firmware, logic/state machines, databases, microprocessors, communication links, displays or other visual or audio user interfaces, printing devices, and any other input/output interfaces, including sufficient fluidic and/or pneumatic control, supply and measurement capability to provide the functions or achieve the results described herein.

FIG. 20A is a flow diagram 2000 for performing loading starter DNA, writing (printing) and unloading coded polymers in an inkjet writing system, in accordance with embodiments of the present disclosure, which logic may be performed by the system 1900 of FIG. 19A. In some embodiments, the above writing process may be repeated for each new set of DNA with beads to be written.

In particular, the logic 2000 begins at block 2002 by loading or printing starter DNA strands onto the wafer array spots. Next, block 2004 it receives the Binary Code to print/write for the current memory string (or nacket). Next, block 2006 performs a wash cycle across the wafer array to clear any extraneous reagents from the surface of the wafer. Next, block 2008 writes/prints the code to the memory string/nacket with the appropriate cassettes at the desired spot(s) per a writing process described herein with FIG. 20B. After the code is written, the block 2010 determines whether there are more spots to be written before the Deblock/Adapter is applied to the spot. If Yes, the logic goes back and writes/prints the code to the memory string/nacket with the appropriate cassettes at the desired spot(s) per a writing process described herein with FIG. 20B until all desired spots are written for that code. Then, block 2012 waits for the addition reaction to complete. When the reaction has completed, block 2014 prints the Deblock/Adapter for the desired spots. In some embodiments, the Deblock/Adapter may be washed across the surface of the array instead of using an inkjet cartridge or head for the Deblock/Adapter. Next, block 2016 determines whether all codes have been written for the current string or nacket. If not, block 2018 gets the next code in the string and proceeds back to the wash cycle and repeats the process for the next code. When all the codes have been written, block 2020 determines whether all memory strings or nackets have been written for the wafer array or chip. If Not, block 2022 gets the next desired the next memory string or nacket to be written and proceeds back to retrieve the binary code to be written, and the logic repeats the process for writing the next desired binary code until all usable desired spots are written on the wafer array or chip, or all desired binary odes have been written. Next, block 2024 washes the wafer array with cleaving fluid and unloads and captures the DNA/polymer memory strings or nackets in a containment bin (for future reading), such as that shown in FIG. 11A, and the logic exits.

FIG. 20B is a flow diagram 2050 for performing writing (printing) a bit or code to a polymer in an inkjet writing system, in accordance with embodiments of the present disclosure, which logic may be performed by the system of FIG. 11A. In particular, the logic 2050 begins at block 2052 which determines if a 0 bit is to be written or added. If Yes, block 2054 causes the appropriate inkjet cartridge or head to print the 0 bit code at the desired spot(s)/locations(s) on the wafer array or chip. Next, or if the result of block 2052 is No, block 2056 determines if a 1 bit is to be written or added. If Yes, block 2058 causes the appropriate inkjet cartridge or head to print the 1 bit code at the desired spot(s)/locations(s) on the wafer array or chip. Next, or if the result of block 2056 is No, block 2060 determines whether bit writing is complete for the spot or group of spots to be written. If No, the logic goes back to block 2052 and determines which code and location to write. If the result of block 2060 is Yes, the appropriate code has been written for the desired number of spots and block 2062 determines whether any droplet errors were detected by the droplet viewer (or sensor), which may be part of the print head and array stage controller and inspection logic. If any errors were detected, block 2064 saves the error location(s) and bit number for future reading, and the logic exits. If the result of block 2062 is No, then no errors were found and the logic exits.

Referring to FIG. 23A, a side cross-section view of a patterned wafer substrate is shown having a hydrophobic coating (right image) and showing a process for using silanization to create the hydrophobic coating, in accordance with embodiments of the present disclosure. In particular, the left side 2302 of FIG. 23A shows an embodiment of a patterned substrate and a blow-up 2304 of the upper surface of a pillar or spot 14, which also shows the chemistry used to attach to the starter DNA/polymer strings or strands (or acceptors) 210, such as Azido-C12-phosphonic acid or 12-azidododecylphosphonic acid (Sikemia). Also, the right side 2310 of FIG. 23A shows an embodiment of the patterned substrate of the left side 2302 and a blow up 2312 of the upper surface of the pillar or spot 14 with a hydrophobic layer 410, e.g., 1H,1H,2H,2H-perfluorooctyltriethoxysilane, surrounding the Sikemia attachment area 204 on the upper surface of a pillar, which may be used to attach to the starter DNA strands (or strings or acceptors).

Referring to FIG. 23B side view images of patterned wafers with two different hydrophobic silane coatings, PFOTES (1H,1H,2H,2H-perfluorooctyltriethoxy-silane) above the dashed line, DDTMS (Dodecyltrimethoxy-silane) below the dashed line, are shown at 3 different temperatures, in accordance with embodiments of the present disclosure. In particular, for the PFOTES, it is shown with Water at 112 deg. F., with Ink Jet Buffer+Tween at 85 deg., and with Ink Jet Butter+Ecosurf at 70 deg. For the DDTMS, below the dashed line, side view images of patterned wafers are shown with Water at 105 deg, with Ink Jet Buffer+Tween at 65 deg., and with Ink Jet Butter+Ecosurf at 35 deg. It also shows analysis of contact angle to show surface hydrophobicity.

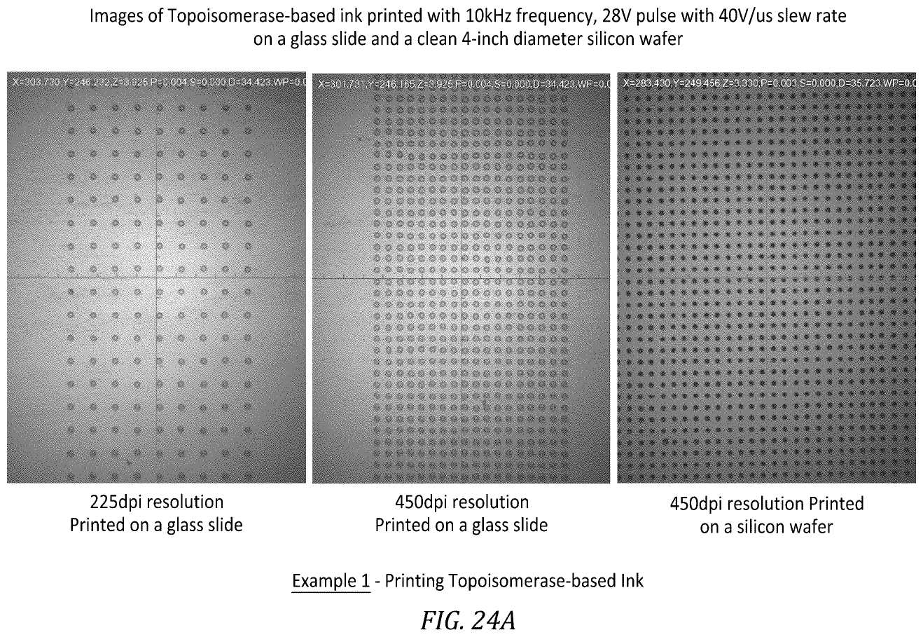

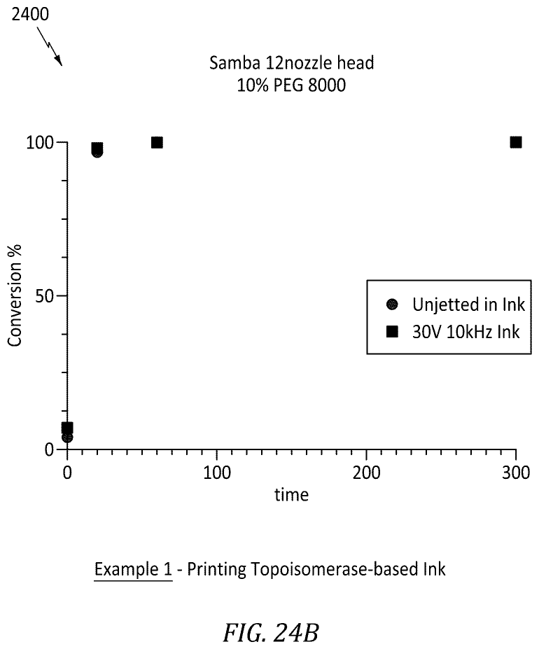

Referring to FIG. 24A, three images for an example of topoisomerase-based ink, with two images printed on a glass slide with two different resolutions is shown and one image on a clean silicon wafer, in accordance with embodiments of the present disclosure. In particular, it shows images of Topoisomerase-based ink printed with 10 kHz frequency, 28V pulse with 40V/us slew rate on a glass slide (left two images) and a clean 4-inch diameter silicon wafer (right image) having 225 dpi resolution (glass slide), 450 dpi resolution (glass slide), and 450 dpi resolution (silicon wafer).

Referring to FIG. 24B, a graph 2400 for the example of FIG. 24A of conversion % vs time is shown, comparing ligation kinetic performance of jetted and unjetted topoisomerase-based ink at four different time points, in accordance with embodiments of the present disclosure. In particular, it shows performance at time points 0, 20 seconds, 60 seconds, and 5 minutes (300 seconds). For the jetted data, printing was done with 30V 10 KHz inkjet with a samba 12 nozzle head 10% PEG 8000.

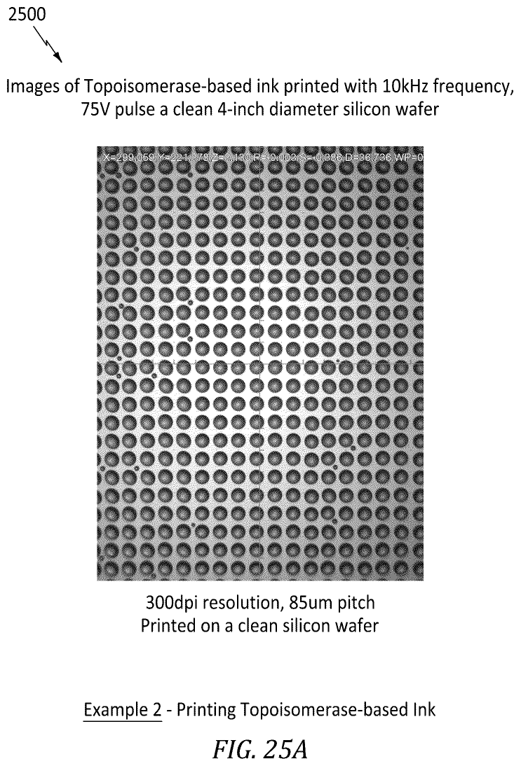

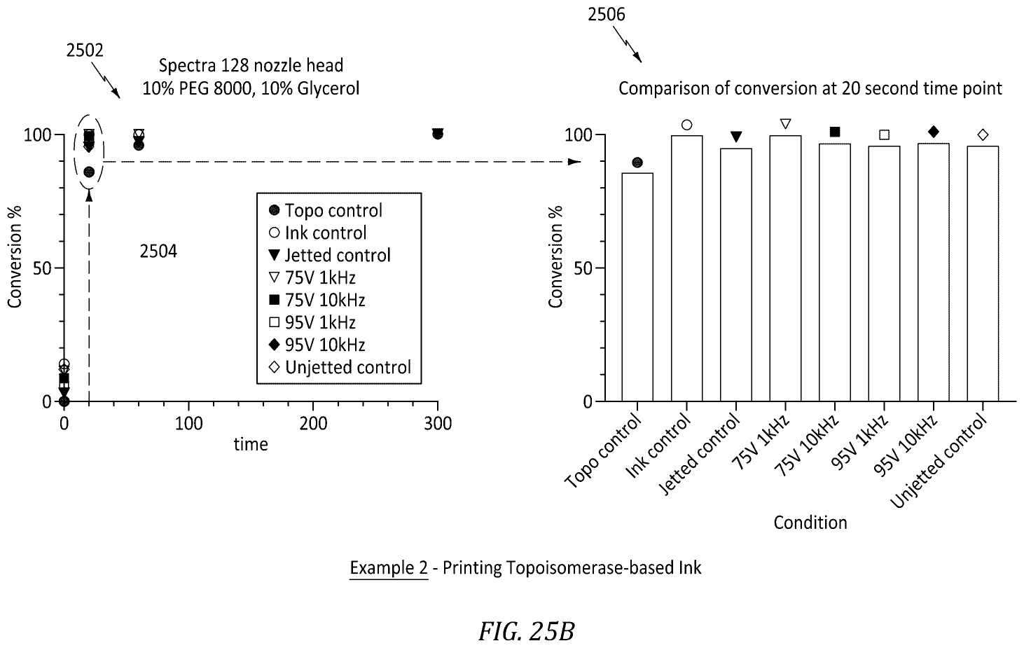

Referring to FIG. 25A, an image 2500 for another example of topoisomerase-based ink printed on a glass slide with 300 dpi resolution is shown, in accordance with embodiments of the present disclosure. In particular, it shows images of Topoisomerase-based ink printed with 10 kHz frequency, 75V pulse, on a clean 4-inch diameter silicon wafer, showing 300 dpi resolution and 85 micron pitch, printed on a clean silicon wafer.

Referring to FIG. 25B a graph 2502 for the example of FIG. 25A of conversion % vs time, comparing ligation kinetic performance of jetted and unjetted topoisomerase-based ink at four different time points is shown, and a blown-up detail comparison bar graph 2506 of conversion at a 20 second time point, in accordance with embodiments of the present disclosure. In particular, it shows performance at time points 0, 20 seconds, 60 seconds, and 5 minutes, using Spectral 128 nozzle head 10% PEG, 10% Glycerol, and for 8 different types of points, as shown in the detail comparison bar graph at the 20 second time point.

Referring to FIG. 26A, a diagram showing an example of bonding and linking chemistry for printing topoisomerase ink on a patterned wafer, with 2608 and without 2604 a hydrophobic coating 410, in accordance with embodiments of the present disclosure. In particular, it shows 12-azidododecylphosphonic acid (Sikemia) attached to the HfO₂, with copper-free click chemistry between the azide moiety on the phosphonic acid and an ADIBO moiety attached to the DNA acceptor strand via an amidoalkyl linker shown collectively as 2602. It also shows the surface with a hydrophobic coating 410, e.g., a perfluorinated alkyl linked to a silyl group linked to the substrate, e.g. a 1H,1H,2H,2H-perfluorooctyltriethoxy-silane. The region 2604 is shown blow up in 2608 as shown by dashed line 2610.

Referring to FIG. 26B, consecutive images 2600 from a cycle of five prints of topoisomerase ink on a patterned wafer is shown for the example of FIG. 26A, in accordance with embodiments of the present disclosure.

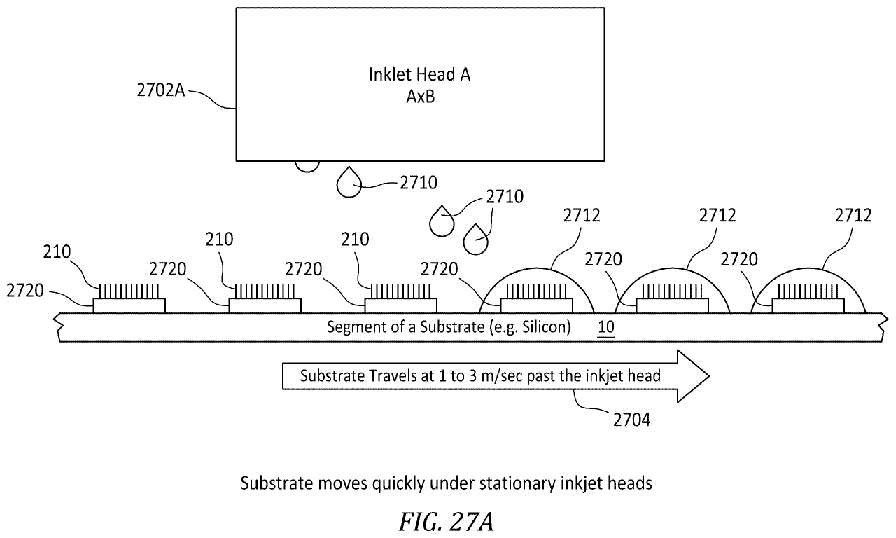

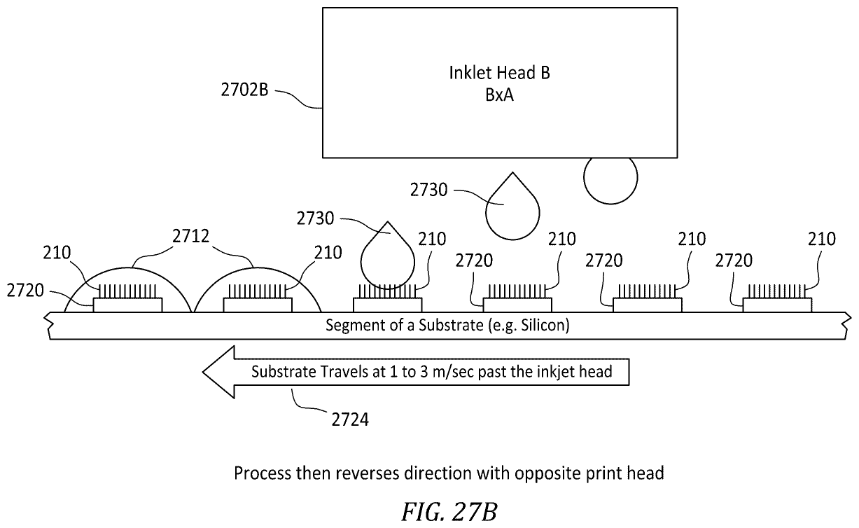

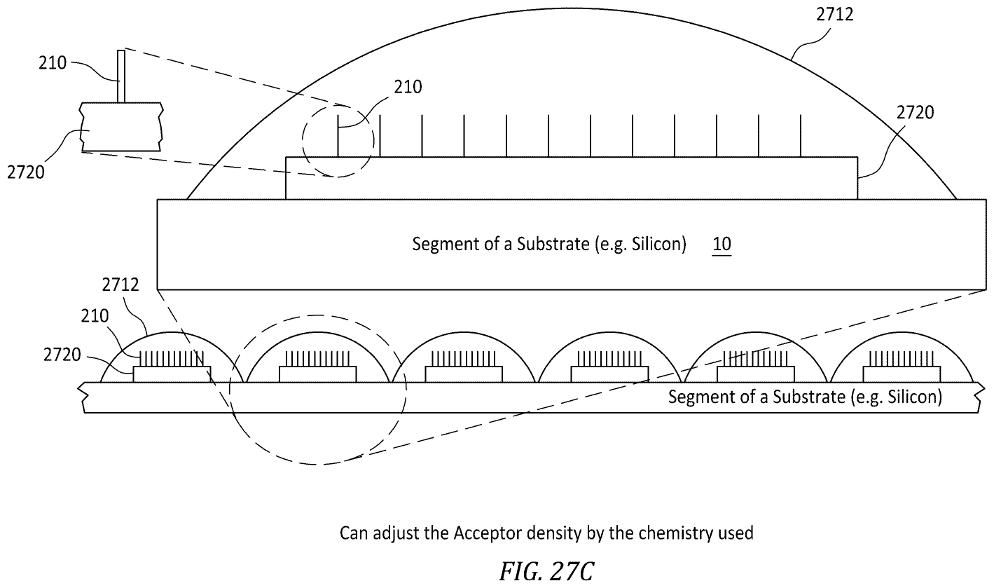

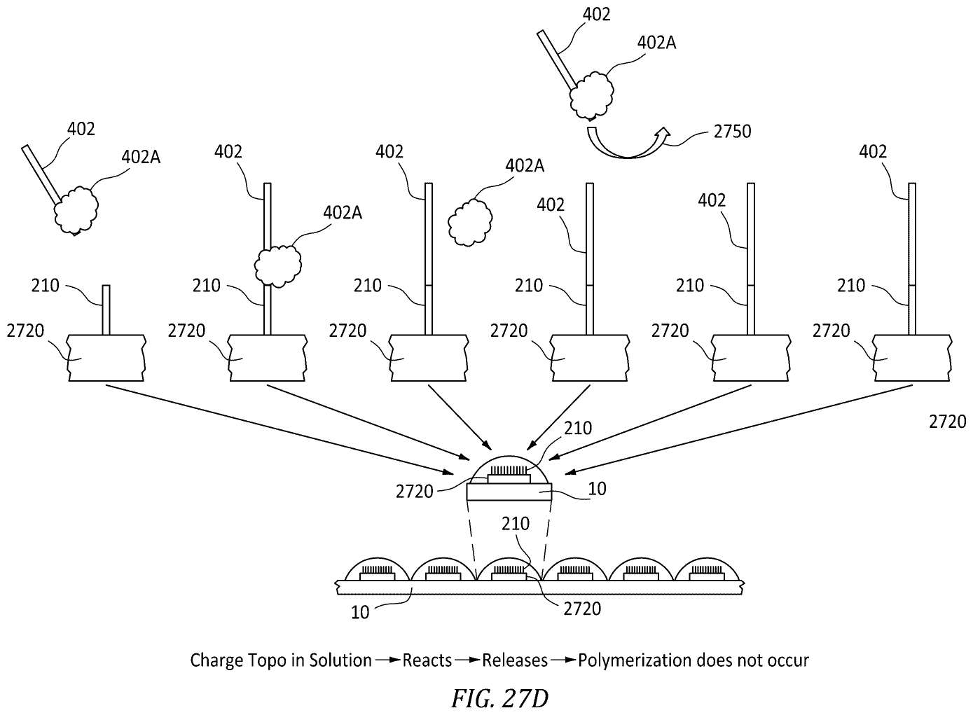

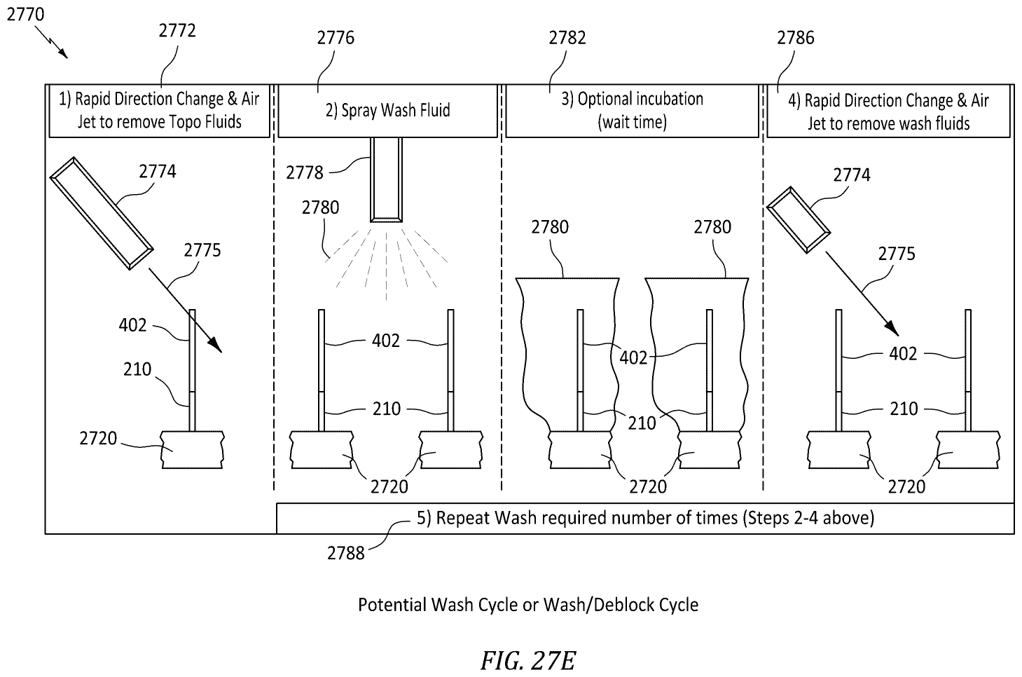

Referring to FIG. 27A, a side view of a flat substrate with pattern spots 2702A with acceptors 210 for synthesis is shown, the substrate 10 moving under a stationary inkjet head A 2702A in a first direction 2704, in accordance with embodiments of the present invention. In particular, the substrate 10 moves quickly past the inkjet head 2702A at about 1 to 3 m/sec. Other speeds may be used if desired. The registration or alignment of the substrate to the inkjet head 2702A may be done with an online optical micrometer to ensure proper alignment or may use other alignment techniques. In some embodiments, a high-resolution system may be up to 1600 dpi (or a pitch or spot spacing of 16 microns) or higher; however, other spacings may be used if desired, such as 64 microns spacing or other spacings. As the substrate moves (or travels) past the inkjet head 2702A, the droplets 2710, e.g., 1.2 to 2.1 pL with 5 to 20 drops per cell (or spot) of addition reagents (“add” reaction fluids), are released and land on the substrate shown by bubbles 2712, which cover the spots or reaction areas 2720. In some embodiments, the inkjet head 2702A may use nozzle redundancy, e.g., 2×, 3×, 4×, 5× or more or other redundancy, to ensure that every spot (or substantially every spot) receives sufficient fluid to allow the desired addition reaction to occur. In some embodiments, the inkjet head 2702A may also move to facilitate the desired droplet 2710 placement on the wafer. Also, in some embodiments, there may be a plurality of inkjet heads (or bank of heads) along the path traversed through substrate. Also, in some embodiments, the wafer 10 may be stationary, and the inkjet head moved or positioned (as discussed herein) to facilitate the desired droplet placement on the wafer(s).

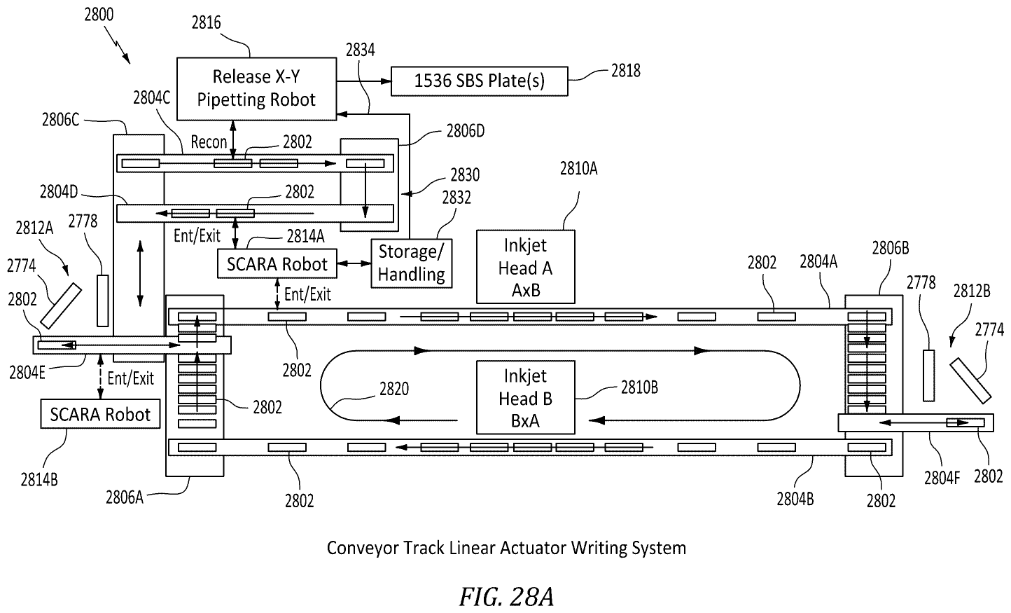

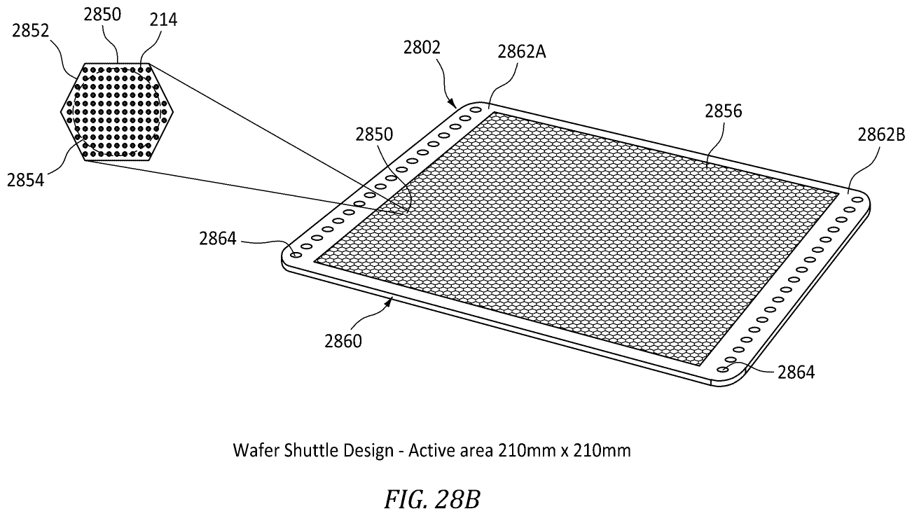

Referring to FIG. 27B, a side view of a flat spot-patterned substrate 10 of FIG. 27A, the substrate 10 moving under a stationary inkjet head B 2702B (which may be a different head from the head in FIG. 27A) is shown moving in a reverse direction from that of FIG. 27A, in accordance with embodiments of the present invention. In particular, the process is similar to that shown in FIG. 27A but with a second writing fluid 2730 (or “add” reaction fluid). Also, in some embodiments, there may be a plurality of inkjet heads for inkjet head B 2702B (or bank of heads) along the path 2724 traversed through substrate 10. There may be a separate track for the second head, such as that shown in FIG. 28A. This “pipelines” (or queues up) the substrates (or wafers) so there are always substrates (or wafers) passing under both inkjet writing heads and the adjacent steps (not shown) (e.g., air/wash cycles, see FIG. 28A) may be parallelized to match the desired throughput.

In some embodiments, the writing approaches described in the present disclosure may be performed with a flat substrate as shown in FIGS. 27A-27C, or may be used with the etched patterned substrates shown in FIGS. 1-3 herein, having the elevated pillars as discussed herein.

Referring to FIG. 27C, a side view of the substrate of FIG. 27A showing details of cell (or spot) 2720 sizes, dimensions and acceptor (or starter DNA strands) density is shown, in accordance with embodiments of the present invention. In particular, the cell (or spot) 2720 spacing may be 32 microns, 64 microns or any other desired spacing that meets the desired function and performance. Also, the diameter of the spot may be 25 microns, 50 microns diameter or any other desired diameter (or equivalent area in another shape) that meets the desired function and performance. Also, in some embodiments, the density of acceptors (or starter DNA strands) on the surface for a given spot or cell 2720 may be 1.0e4 (or 10K) acceptors, or 3.0e4 (or 30K) acceptors, or any other desired acceptor density that meets the desired function and performance and the density of the acceptors may be adjusted by the chemistry used.

Referring to FIG. 27D, a side view of the substrate of FIG. 27C showing a process for a topogation reaction on the surface of the cell (or spot or reaction area or reaction spot) 2720 on the substrate 10 is shown, in accordance with embodiments of the present invention. This illustrates that for a given acceptor, only one topo enzyme 402A will attach to one acceptor 210 leaving a coded DNA strand 402 attached to the acceptor 210, and other topo enzymes will not attach, as shown by the arrow 2750.

Referring to FIG. 27E, steps for an embodiment of a wash cycle 2770 is shown, the wash cycle 2770 to be performed after a topogation addition reaction is completed, in accordance with embodiments of the present invention. In some embodiments, the wash cycle or process 2770 may include a first step 2772 using a rapid direction change and air jet 2774 (or air blades) to blow air 2775 to eject or remove unattached or unbonded or free floating Topo addition fluids/reagents from the surface, then followed by spraying with a spray head 2778 a wash fluid (or buffer) 2780 over the reaction spots 2710 or across the entire, or a predetermined portion of, the wafer or substrate 10 being written. In some embodiments, there may be an optional incubation or waiting time step 2782 if desired, to allow the wash fluid 2780 to remove unattached topo or enzymes from the surface from the writing cycle. In some embodiments, the wash cycle may be combined or alternated with a deblock or adapter cycle. In that case, the wash fluid (Steps 2 and 3 above) 2776, 2782 may change or alternate with each pass through the cycle, e.g., wash fluid, deblock/adapter fluid, wash fluid, etc. Next, there may be another air blade step 2786 to remove the remaining wash fluid 2780 similar to the first step 2772. The wash cycle process 2770 (or a portion thereof, e.g., steps 2 through 4) may be repeated as many times as necessary to prepare the surface for the next addition reaction as shown in step 2788.

Referring to FIG. 28A, a side view of a conveyor track, linear actuator DNA/polymer inkjet writing system 2800 for moving (or shuttling) portable inkjet-writable silicon wafers or wafer shuttles 2802, e.g., via electromagnetic tracks 2804A, 2804B, 2804C, 2804D, 2804E, 2804F (collectively referred to herein as 2804) and/or queue elevators (2806A, 2806B, 2806C, 2806D (collectively referred to herein as 2806), through inkjet head writing stations 2810A, 2810B and washing/drying stations 2812A, 2812B, in accordance with embodiments of the present invention. The system 2800 allows wafers 2802 to enter or exit via an enter/exit queue/elevator 2806A and a load/unload conveyors (left side) 2804C, 2804D using a fluidic release X-Y robot 2814 and air/wash conveyor 2804E. In one embodiment, a wafer (or wafer shuttle) 2802 may enter or be loaded from the left side via the left air/wash conveyor 2804 and on XY Robot 2814B. The wafer 2802 may be inserted into a writing loop 2820 which has two writing conveyors or tracks (upper/lower) 2804A, 2804B and two queue/elevators (left/right) 2806, 2806B, each writing conveyor has an inkjet head 2810A, 2810B, e.g., Inkjet Head A 2810A (along the upper track) and Inkjet Head B 2810B (along the lower track), to add the desired code or cassette to the desired spots on the wafer 2802. In some embodiments, Inkjet Head A 2810A and Inkjet Head B 2810B may each be a bank or plurality of inkjet print heads, as discussed herein. The air/wash conveyor 2804E moves wafers 2802 in or out of the writing loop 2820 based on what codes or cassettes need to be written to which spots on which wafers. After each addition reaction, the air/wash conveyor 2804E, 2804F may remove the wafer 2802 from the writing loop 2820 and perform a wash cycle (e.g., air blade 2774 and wash station 2778), such as described with FIG. 27E discussed herein. In some embodiments, Air/Wash Conveyors 2804E, 2804F accelerate the shuttles 2802 away from the writing loop 2820 and then quickly change direction to create inertial forces that cause surface liquid to be removed from the substrate surface (similar to a vortex spinner or shaker). When the writing process is completed for a given wafer 2802, the enter/exit queue/elevator 2806D may pull the wafer 2802 from the air/wash conveyor 2804E to the separate load/unload conveyor loop 2830 which passes by a release X-Y robot 2816 which may selectively remove the written or coded polymers/DNA from the wafers and place them in a storage container 2818, as discussed herein with FIGS. 11A and 19A.

In that case, the fluidic release X-Y robot 2816 may dispense a cleaving (or releasing) fluid onto each 2802 shuttle or fluidic zones within the shuttle 2802, sucks or washes off coded polymers/DNA and ports them fluidically to multi-well microplate(s) 2818 or other storage container. In some embodiments, the release X-Y robot 2816 may also clean and/or recondition substrate surface and add new acceptors (or starter polymer/DNA strands), when done in line without removing the shuttle 2802 from the conveyor or track 2804C. The load/unload conveyor/loop 2830 may run at a slower rate than the writing loop 2820 to allow for the fluidic release and recondition process to occur while leaving the shuttles 2802 on the conveyor track 2904C, or to allow the removal and replacement of the shuttle on the conveyor track 2904C. When a new or cleaned shuttle 2802 is replaced on the track 2804C, a return elevator 2806D moved the shuttle to the upper load track 2804D which feeds the load/unload elevator 2806C, which completes the load/unload loop 2830.

In some embodiments, instead of cleaning and/or reconditioning the substrate surface, the shuttle 2802 may be removed from the load/unload conveyor loop by a SCARA robot 2814A, or pick and place robot, or other robot, which can pick off or extract the used written shuttle/wafer 2802 and replace it with a clean writable shuttle/wafer 2802. This may be done from the upper load/unload loop 2830, the Air/Wash Conveyor 2804E using the SCARA Robot 2814B, or the upper and/or lower writing conveyors 2804A,2804B, or from anywhere in the writing loop 2820. In some embodiments, the SCARA robot may perform a “hot swap” while the loops are running. The SCARA robot 2814A,2814B may provide the shuttle 2802 to a storage/handling system which receives the shuttle from the robot 2814A and places in a storage container 2832 for later fluidic release or to the fluidic release robot 2816, as shown by a line 2834 for fluidic removal or release and fluidic storage of the coded DNA, as discussed herein.

Referring to FIG. 28B, a perspective view of the portable inkjet-writable silicon wafer shuttle 2802 of FIG. 28A is shown, in accordance with embodiments of the present invention. In particular, in some embodiments, the wafer shuttle 2802 may be a silicon wafer etched with shallow (e.g., 1-2 microns) fluidic wells (or regions or zones) 2853 for fluidic release (or fluidic release zone or release zone), each fluidic release zone 2850 having patterned reaction spots or cells 214 (flat surface or pattern-etched surface) for coded polymer or DNA growth as described herein. The fluidic release zones 2850 may be hexagonal shaped with a wetting boundary or walls 2852 which may be physical/structural (e.g., recessed or raised by about 1 to 2 microns, other heights or depths may be used) and/or chemical (e.g., hydrophobic boundaries, as discussed herein). The size of the release zones 2850 may be 2 mm across, 1K spots, 64 um spacing or 90 mm across, 80K spots, 32 um spacing or any other dimensions, spacing or spot density desired provided it provides the desired function and performance. The shape of the fluidic release zones 2850 may be hexagonal (as shown in FIG. 28B) or may be square, rectangular, circular, oval, trapezoidal, parallelogram, or any other shape that provides the desired function and performance. In operation, in some embodiments, fluidic release zones 2850 may be configured to receive a pipette tip 2854 (shown as a circle in the zoomed in drawing) from a robot, such as the fluidic release robot 2816 shown in FIG. 28A. The pipette tip 2854 may dispense cleaving (releasing) fluid onto each shuttle or zones 2850 within shuttle; sucks or washes off coded polymers/DNA (and may port them to multi well microplate(s) or other storage); and may also clean and/or recondition substrate surface and adds new acceptors to make the shuttle 2802 ready to write additional coded polymers or DNA.

In some embodiments, the writable wafer or substrate 2856 in the shuttle 2802 may be a passive growth substrate (i.e., no electrodes or electronics), which keeps the fabrication costs low, and enables easy update of the growth surface in the field as part of ongoing service or upkeep of the wafer shuttles. In some embodiments, the wafer shuttle 2802 may have an active wafer writing area of about 210 mm×210 mm, which is mounted to or part of a rectangular, stainless steel, non-ferromagnetic frame. Other dimensions and materials for the wafer and wafer shuttle may be used if desired, provided they provide the desired function and performance.

The outer edges on two opposite sides 2862A, 2862B of the frame 2860 may have evenly-spaced ferromagnetic inserts or plugs 2864 that allow the shuttle frame 2860 to be manipulated or moved using electromagnetic controls in the electromagnetic tracks 2804 described herein above with FIG. 28A. Other shapes and materials may be used for the wafer shuttle 2802 if desired, provided it provides the desired function and performance.

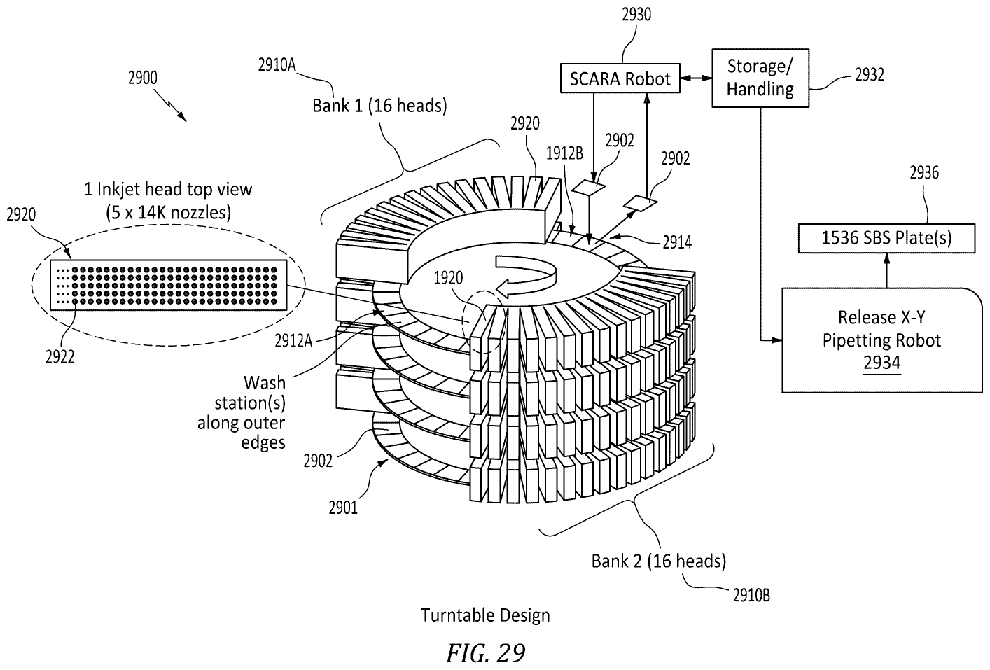

Referring to FIG. 29, a perspective view of a stacked rotary disk (or platen) based turntable inkjet writing system 2900 for inkjet-writable silicon wafer disks 2901 is shown, where the outer edges of the writable wafer disks 1901 having a plurality of wafers or shuttle 2902 disposed on the outer edge of the disk 1901 and are rotated through inkjet head writing stations 1910A, 1910B and washing stations 1912A, 1912B and removal stations 1914, in accordance with embodiments of the present invention. The inkjet head writing stations (or banks) 1910A, 1910B, may comprise a plurality of heads 1920, e.g., 16 heads, per station or bank. Each of the writing heads 1920 may have a plurality of nozzles 1922 with some redundancy, e.g., 5 rows with 14 k nozzles per row, each head 1920 providing an addition reagent (Add “0” or Add “1”, or Add “A0B” or Add “A1B”, and/or a deblock or adapter reaction, or Add “A0B” or Add “A1B”, and Add “B0A” or Add “B1A” without a deblock reaction, as described herein.

In some embodiments, Bank 1 (Add “A-B”) 1910A may have 16 heads 1920 representing 16 different oligos or chemicals (or cassettes) to be added representing or 4 binary bits, and Bank 2 (Add “B-A”) 2910B may have 16 heads 1920 representing 16 different oligos or chemicals (or cassettes) to be added representing 4 binary bits. One byte may be 8 bits, so in some embodiments, one full revolution would result in adding two (4-bit) oligos, which adds 8 bits (or one byte) of data, as shown in the below table. Such a double-sided writing approach may be performed using separate selectable print heads (as shown in FIG. 29), which allows for separate selectable adapter reactions to each add its own unique set of bits, thereby doubling the data storage capability for each write cycle. Such an approach may not be as easily achievable in some other designs that are size constrained, e.g., chip-based designs, where the coded polymer/DNA returns to a common deblock or adapter chamber during each write cycle, as discussed in the aforementioned patent applications and patents.

Bank 1 (2910A) (16 print heads)—Left side—writes “A-B”

A-0000-B
A-0001-B
A-0010-B
A-0011-B
A-0100-B
A-0101-B
A-0110-B
A-0111-B
A-1000-B
A-1001-B
A-1010-B
A-1011-B
A-1100-B
A-1101-B
A-1110-B
A-1111-B

Bank 2 (2910B) (16 print heads)—Right side—writes “B-A”

B-0000-A
B-0001-A
B-0010-A
B-0011-A
B-0100-A
B-0101-A
B-0110-A
B-0111-A
B-1000-A
B-1001-A
B-1010-A
B-1011-A
B-1100-A
B-1101-A
B-1110-A
B-1111-A

Between the two inkjet head banks 2910A, 2910B along the outer rim of the disks, there may be a wash stations 2912A, 2912B, which may provide the air/wash capability discussed hereinabove with FIG. 27E. The circular turntable design of FIG. 29 may provide simpler controls and better position control than a linear conveyor-type system, such as that shown in FIG. 28A, it may also take up less overhead space for handling and provide better accessibility, which may be good for servicing. Also, in some embodiments, the wafer or shuttle 2902 may be removed from the disc by a SCARA robot 2930, or pick and place robot, or other robot, which can pick off or extract the used written shuttle/wafer 2902 and replace it with a clean writable shuttle/wafer. In some embodiments, the SCARA robot 2930 may perform a “hot swap” while the system is running. The SCARA robot 2930 may provide the wafer or shuttle 2902 to a storage/handling system 2932 which receives the shuttle and places it in a storage container for later fluidic release or to a fluidic release robot 2934 for fluidic removal or release and fluidic storage 2936, as discussed herein.

In some embodiments, there may be four (4) rotary mask disks or platens 2901 for a given rotary stage. Other numbers of disks 2901 may be used if desired. In some embodiments, a disk handling system 2900 may control the rotation direction and/or speed of the disk 2901. In some embodiments, a disk handling system may handle releasing or unloading a given wafer or shuttle 2902 into a separate storage area when full and/or loading a new empty wafer or shuttle into the system for writing/storing data.

In some embodiments, an example of the system may have 2.4 m diameter platen 1901 (or disc); 33×˜220 mm square active areas or wafers or shuttles 2902 along outer edges, 16 um active spot size, 90% spot area utilization, 13 RPM platen rotation rate, 300 cassettes, 4-bit fluidic (having 16 different oligos or chemicals to be added for each head bank). The number of wafers may be 30-40 or any other number depending on the size of the platen and the size of the wafers. In some embodiments, the reaction and wash time may be about 1.2 seconds, there may be 4-bit fluidic base (16×2 fluids) and the velocity maybe about 3.3 m/sec (outside). In some embodiments, example racks may include 4 rotary platens with 16 rack footprint, and may use about 6 to 10 more racks for fluidics inputs and controls.

In some embodiments, an example thermal inkjet printing system may comprise a group of 4 print heads having known print head specifications and performance characteristics for printheads or other components that may typically be used with the system, in accordance with embodiments of the present invention. In particular, in some embodiments, a thermal inkjet writing heads and/or system, such as VersaPass™ or DuraLink™, made by Memjet, or the like, may be used in or adapted for some of the embodiments described herein. Based on the desired performance characteristics the data parameters that must be set include: cell spacing, active area, nozzle redundance, module write width, module write speed (m/s), spots across module, spot rate past module (rows/sec), and spot bandwidth (spots/sec), as well as fluid delivery rate. Also, the VersaPass Printheads table provides specifications for a desktop version of the VersaPass printheads including: printhead type, print width, printheads per engine, number of nozzles, nozzle redundancy, drop size, resolution, and print speed. Other models or versions or specifications may be used if desired.

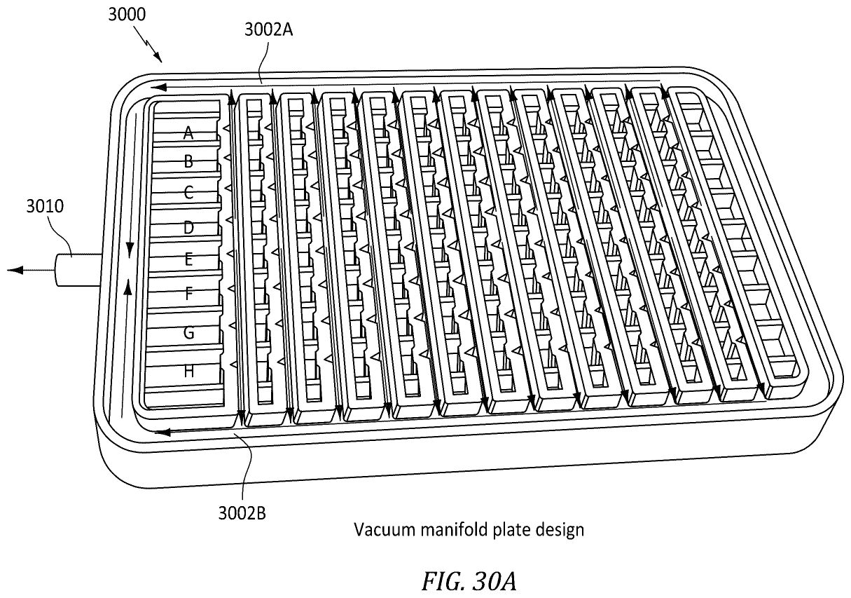

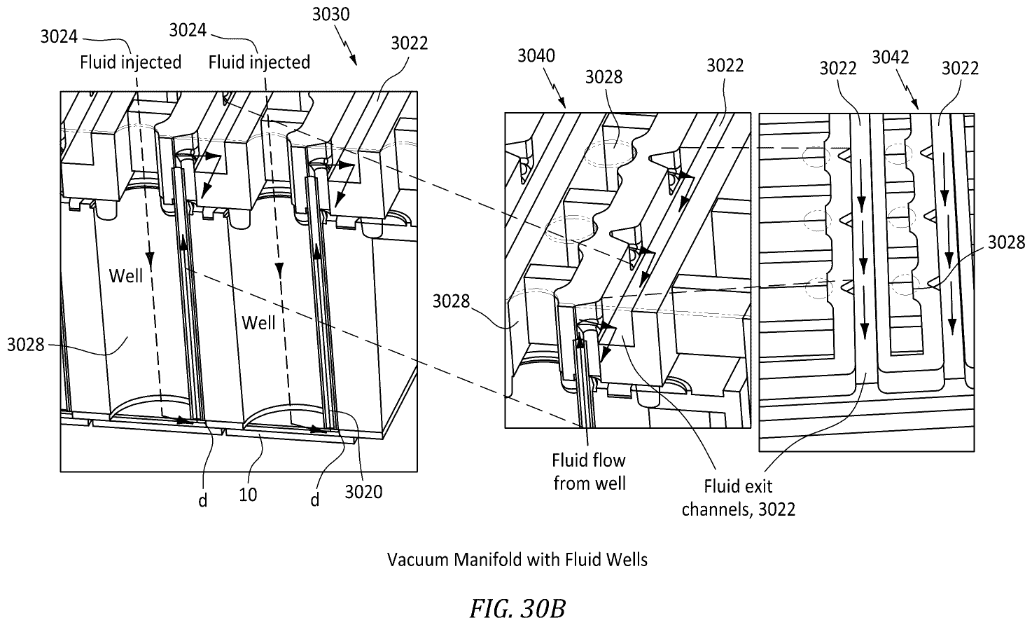

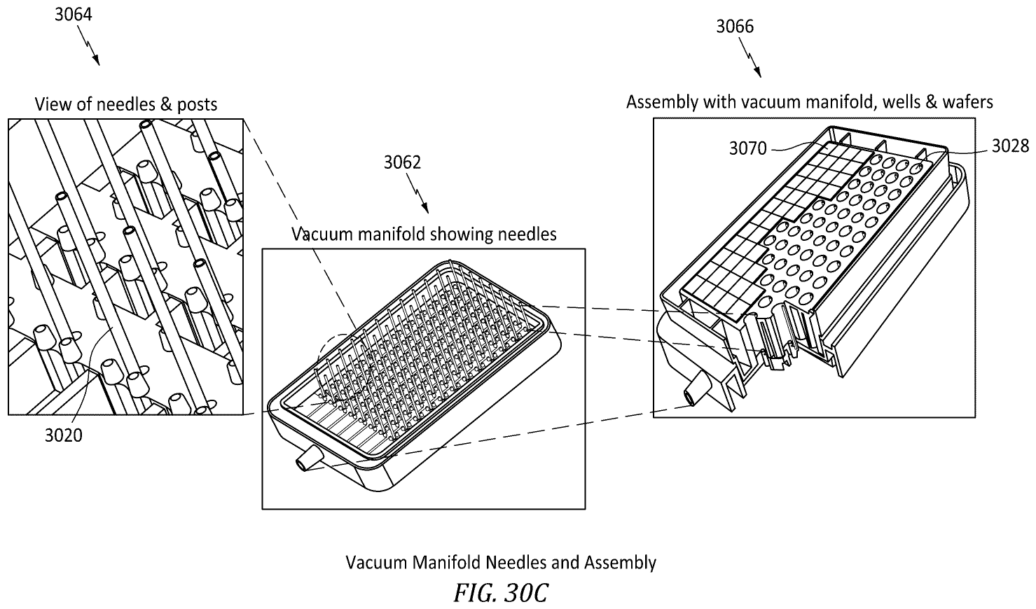

Referring to FIGS. 30A, 30B, 30C, in some embodiments, automated wash and liquid removal from a writing substrate or wafer or shuttle may be performed using a vacuum manifold 3000, which removes or sucks excess unwanted wash fluid or other liquids or fluids from the surface of the substrate, and which may integrate directly with multi-well or microtiter plates.

More specifically, referring to FIG. 30A, a perspective view of a vacuum manifold 3000 plate, that sucks fluid out of each well into a manifold and into a drain system to expedite the wash cycle of a wafer or shuttle, in accordance with embodiments of the present invention. In particular, the vacuum manifold may be placed on top of a 96 well plate, the vacuum manifold having 96 hypodermic needles that suck fluid out of each well into fluid exit channels 3002A,3002B and to an exit port 3010. The manifold 3000 is held at a low pressure so that fluid is sucked into a drain system.

For example, Rows ABCD may drain to the top 3002A and rows EFGH may drain to the bottom 3002B. The top and bottom may drain to the left where there may be an integrated ¼″ ID hose barb (not shown) or port that may be fluidically routed to a container (not shown) that is connected to a vacuum. The vacuum level can be controlled but is not likely to be required at high precision.

A three-way motorized ball valve (or other valve) (not shown) may be used between the manifold and the drain system. When the valve is actuated, the vacuum is dumped, and any residual fluid will remain in the manifold until the next vacuum cycle. Also, gravity helps prevent back flow from the manifold back into the device.

Referring to FIG. 30B, a perspective cut-away view 3040 of the vacuum manifold plate of FIG. 30A, with two close-up views 3042, 3030 of portions of same, which shows fluid flow, in accordance with embodiments of the present invention. In particular, fluid is injected, e.g., using a Dragonfly™ bulk dispenser, low volume, positive displacement, non-contact dispenser, as a jet, shown as dashed down arrows in the drawing into the wells 3028. Topo “add” reagent reaction volumes are low so that a wash cycle can remove residual amount higher in the well 3028. The needles 3028, which on the left close-up view 3030, may have a gap distance d of about 500 microns off the bottom and sucks all the fluid out (other bottom gaps (d) may be used if desired). Fluid flows down the wells 3028 (dashed arrow), then flows in the direction of the solid arrows when being sucked out by the needles 3020 and flows into the fluid exit channels 3022.

Referring to FIG. 30C, a perspective view 3062 is shown of the vacuum manifold of FIG. 30B showing vacuum needles 3020 and a close-up view 3064 showing the needles 3020, and a perspective cutaway view 3066 of an inkjet writing assembly having a vacuum manifold, in accordance with embodiments of the present invention. In particular, the needles 3020 may be glued in place with UV curing adhesive (or any other adhesive or attachment approach) and may have a wicking guide, as shown in the close-up left-most drawing view 3064. Also, the fluid wells 3028 of the well plate may be aligned with the vacuum manifold using posts shown in the close-up view 3064. Also, the far-right drawing 3066 shows an embodiment of a wafer or shuttle assembly having vacuum manifold (on the bottom), well plate with fluid wells 3028, and separate small wafers (on top) 3070.

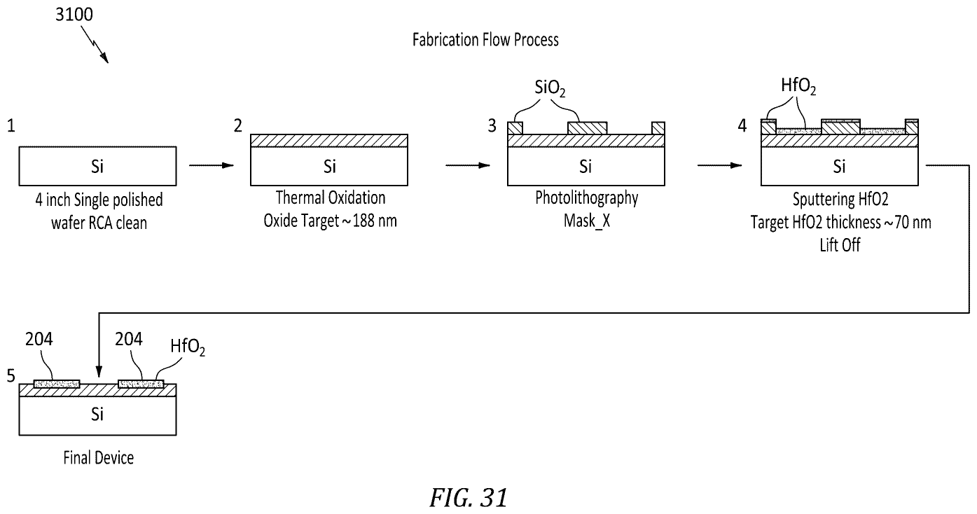

FIG. 31 is a cross-section side view showing an alternative fabrication process 3100 to create patterned wafers, in accordance with embodiments of the present disclosure. In particular, in Step 1, a 4-inch diameter single polished Si wafer RCA clean begins the process. Next, in Step 2, thermal oxidation is performed on the surface with an oxide target of about 188 nm. Next, in Step 3, photolithography is performed with a mask to create SiO₂ layer. Next, in Step 4, a layer of metal oxide is applied or deposited or sputtered to the upper surface of the top layer (hafnium oxide (HfO₂) is preferred, but other metal oxides such as titanium oxide or aluminum oxide could be used alternatively), e.g., ca. 70 nanometer (nm) thick, and then a lift off procedure is performed leaving HfO₂ spots 204 on the surface for the reaction spots, as shown in Step 5. Other approaches or processes may be used to create the same resultant shape and layer or region 204 if desired.

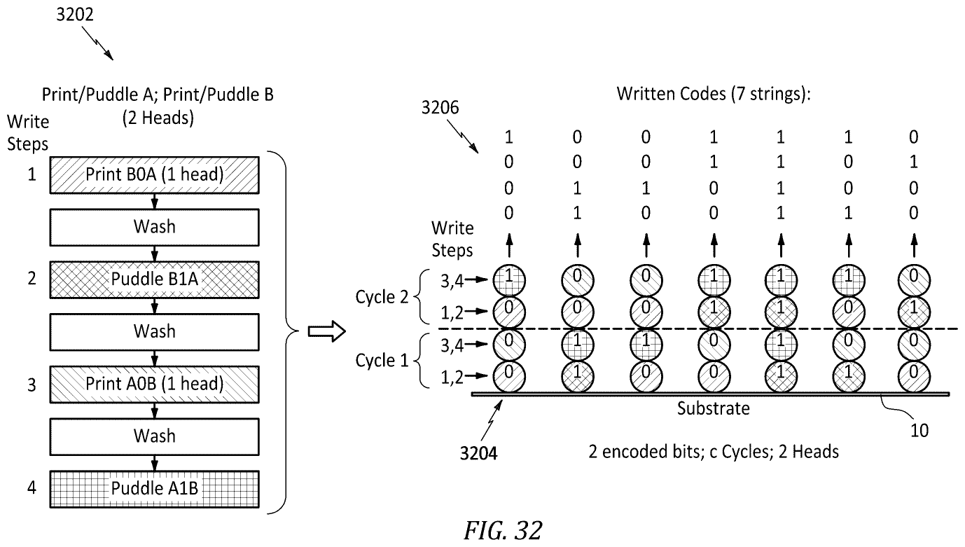

FIG. 32 is a diagram of a dual print head assembly writing process using a print/puddle approach using two print heads, showing two writing cycles and four layers of 1's and 0's being written and corresponding written codes, in accordance with embodiments of the present disclosure. In particular, the left side shows a process 3202 for one writing cycle, having 4 write steps and wash steps in between each write step. In the first write step, the first print head prints B0A to the desired spots on the wafer for a 1. Next, a wash step is performed. The second writing step is a “puddle” writing step where the wafer is placed in a bath or dipped in fluid of B1A to do the addition reaction to add 1 s, not using the inkjet printhead. In that step, any spots that are not filled with a 0, must, by definition, be a 1, and the B1A will only attach to the spots where B0A did not write. Next, a wash step is performed. Next, in the third writing step, the second print head prints A0B to the desired spots on the wafer for a 0. Next, a wash step is performed. The fourth writing step is a “puddle” writing step where the wafer is placed in a bath or dipped in fluid of A1B to do the addition reaction to add 1 s, not using the inkjet printhead. In that step, any spots that are not filled with a 0, must, by definition, be a 1, and the A1B will only attach to the spots where A0B did not write. This completes one cycle of writing for this approach. The diagram 3204 illustrates the results of the above print/puddle process, showing that there are 0 s and 1 s in the first row from writing steps 1 and 2, and there are 0 s and 1 s in the second row from writing steps 3 and 4, both for Cycle 1. Each circle represents a cassette and the 0/1 in the circle represents the value of the bit being written (for single bit binary encoding). If the process is repeated, it will create another pair of rows as shown in the diagram 3204 for Cycle 2. The diagram 3206 above the diagram 3204 shows the resulting binary code being written in the memory string. Other variations of the print/puddle (or print/pool) approach may be used. The benefit of this print/puddle process is 1 bit per cycle is written. Potential cons of this approach are a longer cycle time (e.g., about 6 min/cycle), and there is a potential for crosstalk, which can be mitigated by performing topo inactivation.

The embodiments described in the present disclosure may be implemented using piezoelectric inkjet print head, thermoelectric (or thermal) inkjet printheads or any other type of inkjet head, provided it provides the desired function and performance, including but not limited to delivering the desired fluid droplets to the wafer or substrate or shuttle at the desired reaction spots or cells as described herein. For a thermal inkjet printhead, a small portion of the fluid located away from the nozzle may be electronically vaporized, the vaporized gas creates increased pressure within the head, which pushes the fluid out of the nozzle at the opposite end of the head.

In some embodiments, the corresponding fluid buffer and reagents discussed herein may be loaded and/or unloaded by a fluidics instrument attached to or part of the inkjet printer system or instrument of the present disclosure. Other configurations may be used for the fluidic circuit if desired, provided it provides the desired function and performance.

In some embodiments, instead of doing the deblock/adapter action using a print head nozzle or chamber, deblocking may be performed on the array using known photo-induced deprotection or deblocking and/or known electrochemical deprotection or deblocking, such as is described in published US patent application US2021/0332351A1, which is incorporated herein by reference to the extent necessary to understand the present disclosure. For electrochemical deprotection or deblocking, the necessary electrodes and voltage controls may be added to the array and/or the instrument to provide such a function. For photo-induced deprotection or deblocking, the necessary optical sources and/or mirrors, such as a Digital Micromirror Device (DMD) and associated components and controls may be added to the array and/or the instrument to provide such a function.

The term “data” as used herein includes all forms of data including data representing addresses (or labels or pointers, including physical or virtual), machine code of any type (including but not limited to object code, executable code and the like), error checking, encryption, libraries, databases, stacks, and the like that may be stored in memory. In certain examples, the term “Data” may be shown or described as being separate from the “Address,” or “Error Checking”. In those cases, these terms may be used to show different forms of data for illustrative purposes only.

The starter DNA (or polymer) strands or strings may be loaded by any process that causes the starter polymer or DNA strand or string to be attached to the desired spots on the wafer array provided it provides the desired function and performance requirements. For example, the starter DNA (or polymer) may be loaded onto the spots before the wafer is put into the inkjet printer, or may be loaded onto the spots by the inkjet printer as discussed herein.

In some embodiments of the present disclosure, the Add nozzles and Deblock/Adapter nozzle may be fluidically connected to one or more respective supply containers which may provide the appropriate fluid and enzymes needed to perform the addition and deblock/adapter reactions, as discussed herein and in the aforementioned commonly owned patents and patent application.

In some aspects or embodiments, the present disclosure provides a method for writing, by at least one writing print heads, a unique code to polymer memory strands dispensed on at least one writing spot on the wafer array, the head or nozzle writing the same code to a plurality of DNA memory strands dispensed on the at least one spot, the method comprising: loading the desired spot to be written with starter polymer or DNA attached to the desired spot; washing the surface of the wafer array; positioning an Add “0” or Add “1” piezo-electric inkjet nozzle having the corresponding Add “0” and Add “1” reagents over a desired spot to be written; causing the piezo-electric inkjet nozzle to release a droplet of the corresponding Add “0” or Add “1” reagent onto the spot, thereby writing a bit or code to the DNA or polymer memory strings (or strands) associated with the spot; washing the surface of the spot; causing the piezo-electric inkjet nozzle to release a droplet of deblock/adapter reagent onto the spot; washing the surface of the spot; when the code writing is complete for the memory strings at the spot, flowing a cleaving fluid over the spot thereby removing the memory strings from the spot and flowing the memory strings from the spot into a collection or storage container for later reading.

In some aspects or embodiments, the present disclosure provides a method for simultaneously writing, by a plurality of writing print heads, unique codes to polymer memory strands dispensed on a plurality of writing spots on the wafer array, each head or nozzle writing the same code to a plurality of DNA memory strands dispensed on a given spot, the method comprising: loading the desired spot to be written with starter polymer or DNA onto the desired spots; washing the surface of the wafer array; positioning an Add “0” or Add “1” piezo-electric inkjet nozzle having the corresponding Add “0” and Add “1” reagents over a desired spots to be written; causing the piezo-electric inkjet nozzle to release a droplet of the corresponding Add “0” or Add “1” reagent onto the spot, thereby writing a bit or code to the DNA or polymer memory strings (or strands) associated with the spot on the wafer array; washing the surface of the wafer array; causing the piezo-electric inkjet nozzle to release a droplet of deblock/adapter reagent onto the spot; washing the surface of the wafer array; when the code writing is complete for all the memory strings at all the spots on the wafer array; washing the surface of the wafer array with a cleaving fluid which removes the memory strings from the spots and flowing the memory strings from the wafer array into a collection or storage container for later reading.

In some embodiments, the method comprises simultaneously writing, by a plurality of writing print heads, unique codes to polymer memory strands dispensed on a plurality of writing spots on the wafer array.

Also, in some embodiments, the spots are patterned on the wafer array using pillars surrounded by a circular channel. Also, in some embodiments, the pillars have a region of HfO₂ to attach the starter polymer or DNA strands.

In some embodiments, the method further comprises washing the wafer array with a preparation fluid before attaching the starter strands to the spots. Also, in some embodiments, the washing may be performed by flowing a washing fluid into an input port or manifold fluidically connected to one side of the wafer array causing the fluid to flow across the wafer surface and to exit an output port or manifold on an opposite side of the wafer. Also, in some embodiments, the washing may be performed by providing a washing print head with a nozzle which dispenses a predetermined amount of washing fluid to each desired spot on a wafer array surface.

Also, in some embodiments, the starter strands or strings may be loaded and attached to the spots by providing a washing print head with a nozzle which dispenses a predetermined amount of starter strands in a fluid to each desired spot on a wafer array surface.

Also, in some embodiments, the starter strings are attached to the spots, then dried and then rehydrated before use in the inkjet printer. Also, in some embodiments, after writing the codes, the coded polymers attached to the spots on the array are then dried and stored, and then rehydrated and removed for reading or storing.

Also, in some embodiments, the method comprises evaluation of printing density using unique molecular identifiers (UMIs). For example, repeated addition rounds using multiple different oligomers which are added randomly to the strands in each round of addition, generates a diversity of sequences, so that the diversity of sequences becomes exponentially greater in each round. So if four different oligomers are added randomly in each round, there are four different strand types after one round, 16 after the second round, and so on. After 10 rounds, the number of sequences would be more than a million (4¹⁰), and after 25 rounds, the number of sequences would be more than 10¹⁵. If the number of possible sequences exceeds the number of DNA molecules, each molecule is predicted to have a unique sequence and thus has a UMI. The DNA sequences can then be released from the substrate and the number of unique DNA sequences cleaved from said printed surface area can be quantified, yielding an approximate number of DNA strands per the area of substrate strands, e.g., um², or per dot on the substrate bearing acceptor strands. For example, grafting cycloalkyne-functionalized (ADIBO- or DBCO-functionalized) acceptor strands onto azido functionalized substrate at a concentration of 1 nM, a UMI analysis (extending the DNA strands by repeated rounds of addition of random combinations of oligomers to obtain a large diversity of sequences) may yield approximately 340 DNA molecules/um²; at a concentration of 5 nM, a UMI analysis may yield approximately 740 DNA molecules/um²; at a concentration of 10 nM, a UMI analysis may yield approximately 1100 DNA molecules/um²; at a concentration of 25 nM, a UMI analysis may yield approximately 2900 DNA molecules/um²; at a concentration of 100 nM, a UMI analysis may yield approximately 8500 DNA molecules/um². This analysis is useful to quantitate number of molecules per nacket and to assess PCR amplification biases/errors.

### Topoisomerase Ligation

In particular embodiments, the DNA strands are synthesized using topoisomerase-mediated ligation of DNA oligomers, or cassettes. Topoisomerases are enzymes that spontaneously recognize and cleave at least one strand of a double strand of nucleic acids within a sequence segment known as the site-specific recombination sequence. For example, Vaccinia topoisomerase is a type I DNA topoisomerase that has the ability to cut DNA strands 3′ of its recognition sequence of 5′-(C/T)CCTT-3′, e.g., 5′ CCCTT 3′, and to ligate, or rejoin the DNA back together again. SFV topoisomerase I recognizes the same sequence as Vaccinia topoisomerase—5′-(C/T)CCTT-3′—and can also recognize the variant sequence 5′-CCCTG-3′. Oligonucleotide cassettes containing digital information can be linked together by topoisomerases. In this approach, the DNA base cassette contains a topoisomerase recognition sequence, thereby allowing it to be “charged” with a topoisomerase, such that a strand of DNA is cleaved by the enzyme, and becomes transiently covalently bound to a topoisomerase at the 3- end. When an appropriate DNA acceptor is found, the topoisomerase ligates the cassette to the DNA acceptor strand in a process referred to as “bit addition” or “topogation”. After ligating the DNA cassette onto a DNA acceptor strand, the topoisomerase is no longer bound to the DNA. The DNA thus formed can be a substrate for further addition, if the 5′ end of the DNA thus formed is not protected. This will allow the addition of more than an oligomer to the acceptor DNA in each cycle of addition. The 5′ end of the oligonucleotide can be protected, e.g., by 5′ phosphate, in order to prevent the addition of more than an oligomer in each cycle of addition. The ability of the 5′-phosphate on the ‘acceptor’ DNA to inhibit the addition reaction is strong enough that the growing DNA chain of the acceptor with 5′ phosphate is not capable of ligation to a Topo-charged cassette, until it is exposed to a phosphatase, which removes the 5′ phosphate.

US20210262023A1, which is incorporated herein by reference in its entirety, describes methods of synthesizing DNA in the 3′ to 5′ direction using topoisomerase. In this method, a DNA molecule is synthesized using topoisomerase-mediated ligation, by adding single nucleotides or oligomers to a DNA strand in the 3′ to 5′ direction, comprising (i) reacting a DNA molecule with a topoisomerase charged with the desired nucleotide or oligomer wherein the nucleotide or oligomer is blocked from further addition at the 5′ end, then (ii) deblocking the 5′ end of the DNA thus formed, and repeating steps (i) and (ii) until the desired nucleotide sequence is obtained. For example, using just two different oligonucleotides or two different single nucleotides, a DNA sequence embodying a binary code can be formed, providing a compact means of information storage. DNA encoding ternary codes or encoding genetic information can be synthesized as well.

In the embodiments described in US20210262023A1, the 5′ end of the DNA base cassette is protected, e.g., by 5′ phosphate, so the DNA formed by topogation cannot serve as a substrate for further addition until the 5′ end is deprotected, thereby preventing uncontrolled addition of multiple cassettes. Before the next addition, the DNA is deprotected, e.g., exposed to a phosphatase where the protecting group is a 5′-phosphatase, to remove the protecting group.

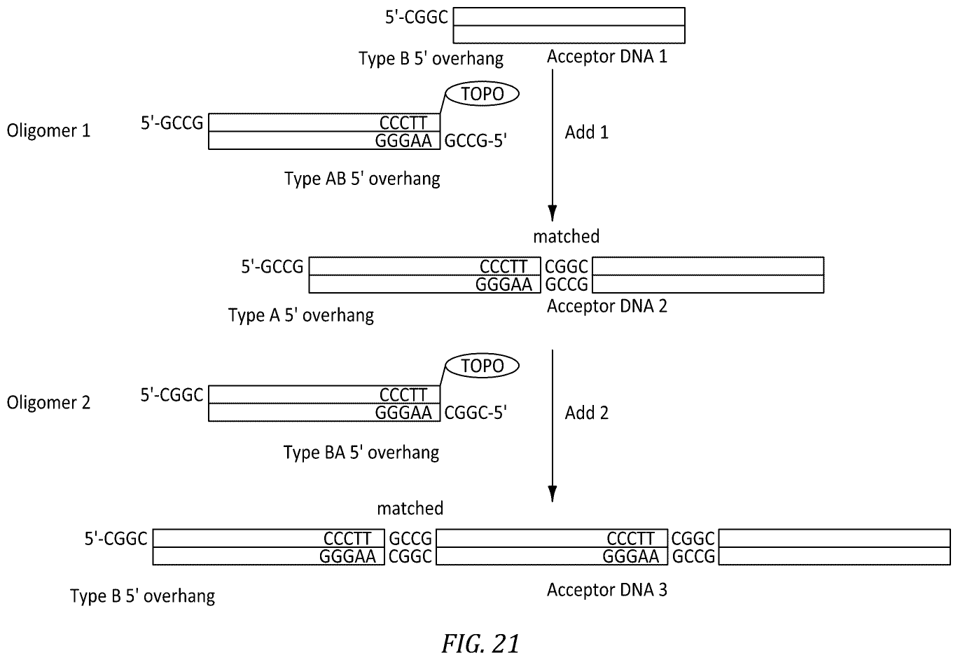

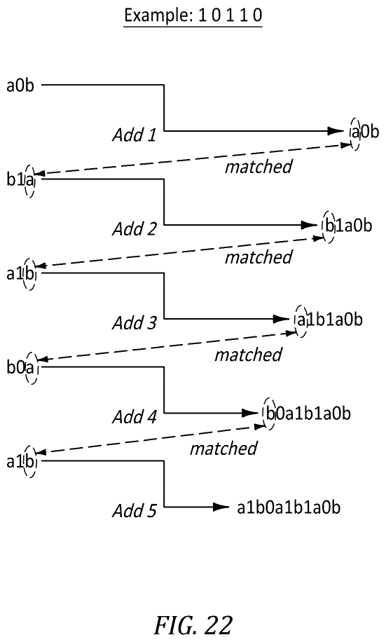

U.S. Provisional Application No. 63/369,339, filed Jul. 25, 2022, which is incorporated herein by reference in its entirety, describes a phosphatase-free method of topoisomerase-mediated DNA synthesis, wherein the DNA cassette added to the acceptor DNA strand contains an overhang, so that it can only be added to by a cassette having a complementary overhand, as illustrated in FIGS. 21 and 22. The need of the deprotection step is eliminated by using double stranded oligomers having 5′ overhangs on both strands. It has been found that when 5′ overhang of the acceptor DNA is not complementary to the 5′ overhang of the strand (“bottom strand”) complementary to the strand bearing the topoisomerase of the double-stranded donor oligomer, the acceptor DNA is not capable of ligation to the topo-charged oligomer, even if 5′ end of the acceptor DNA is unprotected, e.g., unphosphorylated. Based on this finding, we have developed a method of synthesizing DNA using topoisomerase-mediated ligation without protection/deprotection steps by using double-stranded oligomers having 5′ overhangs on both strands, wherein the two overhangs are not complementary to each other and 5′ ends of the oligomers are not protected, e.g., not phosphorylated.

FIG. 21 shows a scheme of phosphatase-free “bit addition” or “topogation”. In the scheme shown in FIG. 21, 5′ ends of oligomers are not protected, e.g., not phosphorylated. A topo-charged oligomer, Oligomer 1, is reacted with Acceptor DNA 1. Oligomer 1 has 5′ overhang (type A) on the strand bearing the topoisomerase (“top strand”) and 5′ overhang (type B) on the strand (“bottom strand”) complementary to the strand bearing the topoisomerase. In this disclosure, the 5′ overhangs of Oligomer 1 is denoted by “type AB”. The 5′ overhang of Acceptor DNA 1 is complementary to 5′ overhang (type B) of the bottom strand of Oligomer 1. The 5′ overhang of Acceptor DNA 1 is denoted by “type B”. Note that type B overhang of Acceptor DNA 1 is complementary to (but not same as) type B overhang of the bottom strand of Oligomer 1. For example, if the sequence of type B overhang of the bottom strand is 3′-GCCG-5′, the sequence of type B overhang of the top strand is 5′-CGGC-3′. Because the 5′-overhang of the bottom strand of Oligomer 1 is complementary to 5′-overhang of the top strand of Acceptor DNA 1, Oligomer 1 is ligated to Acceptor DNA 1 to form Acceptor DNA 2. The 5′ overhang (type A) of the top strand of the DNA thus formed is not complementary to the 5′-overhang (type B) of the bottom strand of Oligomer 1. Thus, in the first cycle of bit addition, no additional oligomer can be added to Acceptor DNA 2, although the 5′ end of the DNA thus formed (Acceptor DNA 2) is not protected. In cycle 2, a topo-charged oligomer having 5′ overhangs of type BA is added. Because the 5′-overhang (type A) of the bottom strand of Oligomer 2 is complementary to 5′-overhang (type A) of the top strand of Acceptor DNA 2, Oligomer 2 is ligated to Acceptor DNA 2 to form Acceptor DNA 3. The 5′ overhang of the top strand of the DNA thus formed (Acceptor DNA 2) is type B, which is not complementary to the 5′-overhang (type A) of the bottom strand of Oligomer 2. Thus, in the second cycle of bit addition, no additional oligomer can be added to Acceptor DNA 3. This process can be repeated until the desired nucleotide sequence is obtained. In this way, only one oligomer is added to the acceptor DNA in each cycle of bit addition without protection/deprotection steps.

The top strand of oligomers bearing the topoisomerase comprises 5′ overhang, informational sequence and topoisomerase recognition sequence, e.g., 5′-(C/T)CCTT-3′. The 3′ end of the top strand is covalently attached to the topoisomerase. The top and bottom strands of oligomers are complementary to each other except 5′ overhangs in the end of both strands. The DNA polymer synthesized by the methods of the present invention comprises a series of informational sequences, each of which is flanked by a topoisomerase recognition sequence and one of 5′ overhang sequences. In some embodiments, the DNA polymer is designed to store data. In some embodiments, the data is stored in a binary code (1's and 0's). In some embodiments, an easily recognized sequence of two or more bases (e.g., 5′-CCG-3′) corresponds to a 1 and another easily recognized sequence of two or more bases (e.g., 5′-AAA-3′) corresponds to a 0. In other embodiments, the data can be stored in a ternary, quaternary or other code.

For the data stored in a binary code (1's and 0's), DNA polymer can be synthesized using four oligomers: A0B, B0A, A1B, B1A. “A” or “B” on the left and right ends indicates the types of overhangs of oligomers. “0” or “1” indicates the binary code corresponding to the information sequence of oligomers. For example,

A08:
[SEQ ID NO: 6]
GCCGGGCCTCGAAACCCTT *
[SEQ ID NO: 1]
CCGGAGCTTTGGGAAGCCGp
A1B:
[SEQ ID NO: 7]
GCCGGGCCTCGCCGCCCTT *
[SEQ ID NO: 2]
CCGGAGCGGCGGGAAGCCGp
B0A:
[SEQ ID NO: 8]
CGGCCTCGACGAAACCCTT *
[SEQ ID NO: 3]
GAGCTGCTTTGGGAACGGCp
B1A:
[SEQ ID NO: 9]
CGGCCTCGACGCCGCCCTT *
[SEQ ID NO: 4]
GAGCTGCGGCGGGAACGGCp

In this example, the informational sequence (in this case AAA corresponding to “0” and CCG corresponding to “1”, but could be nearly any sequence) is bolded, the topoisomerase recognition domain (in this case 5′-CCCTT-3′) is italicized, the 5′-overhang (in this case, the “A” sequence is CGGC and the “B” sequence is GCCG) is underlined, and the topoisomerase enzyme is indicated by an *. Addition to an acceptor DNA would proceed as depicted in FIG. 21.

FIG. 22 shows a scheme of the phosphatase-free topoisomerase-mediated synthesis of DNA polymer storing binary information “10110”. Type AB or type BA topo-charged oligomers are added to the growing acceptor DNA alternatively. In the example shown in FIG. 22, type AB topo-charged oligomers are added in the 1, 3, and 5 round of bit addition, while type BA topo-charged oligomers are added in the 2 and 4 round of bit addition. Which topo-charged oligomer is added in any given round of bit addition is also determined by the binary information to be stored in the position. For example, in order to store the bit information “1” in the third position, topo-charged oligomer AB is added to the growing acceptor DNA in the third round of bit addition. In some embodiments, the oligomer is selected from four oligomers, e.g., A0B, B0A, A1B, B1A, wherein “A” or “B” on the left and right ends indicates the types of overhangs of oligomers and “0” or “1” indicates the binary code corresponding to the information sequence of oligomers. The information density can be increased by using oligomers comprising more than one bit, e.g., eight different oligomers would provide all possible 2-bit additions: A00B, A01B, A10B, A11B, B00A, B01A, B10A, and B11A.

This AB/BA approach, wherein an “AB” reagent comprising information can only add to a strand having a “B” complementary end and a “BA” reagent can only add to a strand having an “A” complementary end, as illustrated in FIGS. 21 and 22, also allows a “print/puddle” approach using as few as two ink jets, as depicted in FIG. 32. For example, in some embodiments, a first cassette, e.g., one of A0B or A1B, is printed at desired spots, followed by puddle (i.e., fully washing, immersing, dipping, or covering) the substrate with a secondary cassette, e.g., the other of A0B or A1B, adding the other cassette in all places that did not receive a cassette in the first printing step. So, for example, in FIG. 32, an inkjet is used to deposit charged topoisomerase with B0A cassettes in specific spots, washed, then the entire surface is exposed by puddling to charged topoisomerase with B1A cassettes, and washed. The “B1A” cassettes will not react with strands that have already received an B0A cassette. Then an inkjet is used to deposit charged topoisomerase with A0B cassettes in specific spots, washed, then the entire surface can be exposed by puddling to charged topoisomerase with A1B cassettes, and washed. The process—printing with a first AB reagent, washing, puddling with a second AB reagent, washing, printing with a first BA reagent, washing, puddling with a second BA reagent, and washing—can be repeated until the desired sequences are obtained. This approach allows the use of as few as two inkjets (one dispensing topoisomerase charged with B0A, and one dispensing topoisomerase charged with A0B in the example depicted in FIG. 32).

A print/puddle approach can be used to reduce interstitial errors, i.e., DNA strands forming at sites between the desired printing spots, which can contaminate the desired population of DNA strands on the printing spots. For example, in one embodiment, all the print spots are printed with a first cassette, e.g., AB, and then the substrate is puddled with a non-amplifiable/non-extendible cassette, e.g., “A×B”, such that all locations on the substrate that did not receive the first cassette (i.e., any strands in interstitial locations and not on the desired “print” spots) are inhibited from further growth (i.e., capped) by the “A×B” cassette.

In some embodiments, the “print/puddle” method may use the same or different ink compositions in the print and puddle steps. In some embodiments, the printing ink has a higher viscosity than the puddle ink. For example, in one embodiment, the printing ink comprises 10% PEG 8000, 10% glycerol, 500 mM ammonium acetate (NH₅Ac), 20 mM Tris pH 8.0, and DNA-charged topoisomerase, e.g., 2.5 uM charged topoisomerase, while the puddle ink comprises 5% PEG 8000, 500 mM NH₄Ac, 20 mM Tris pH 8.0, and DNA-charged topoisomerase, e.g., 0.5 uM charged topoisomerase. In some embodiments, the printing and/or puddle ink further comprises an inert dye, e.g., ≤0.1% saturated, water-soluble, inert dye, for visualization.

In some embodiments, the DNA-charged topoisomerase within the printing and/or puddle ink comprises a terminal phosphate group during storage, which is removed prior to use. Without being bound by theory, it is believed that the inclusion of a terminal group on the topoisomerase-bound DNA oligomer improves stability of the charged topoisomerase and prevents undesired reaction/polymerization during storage of the ink. To remove the terminal phosphate group from the topoisomerase-bound DNA oligomer prior to use of the printing and/or puddle ink (i.e., to “activate” the charged topoisomerase), magnesium chloride (MgCl₂), e.g., 100 uM MgCl₂, and phosphatase, e.g., calf intestine phosphatase (CIP) and/or shrimp alkaline phosphatase (SAP), e.g., 10 ug phosphatase per 2.5 nmol topoisomerase, are added to the ink. In some embodiments, the ink is mixed and filtered before adding to the printhead.

The number of oligomers required to provide a binary code can also be reduced to as few as three, by using A0B and A1B to provide the “0” or “1” and an BA adapter to provide the function of “deprotecting” the DNA strand after addition of the A0B or A1B. After the addition of the oligomer bit, the end of the strand receives a “BA” adapter, so that it again has an “B” 5′ overhang and can receive either of the oligomer bits, A0B or A1B. In other words, the adapter cassette changes the ‘end’ so that it can be topogated by either of the two bits, a process conceptually similar to the synthesis described in US20210262023A1, but instead of removing a phosphate to deprotect the acceptor strand, the adapter oligomer is added to provide a compatible sequence overhang for the next bit addition, using the exemplary A0B and A1B sequences above, and a BA adapter, e.g.,

A08:
[SEQ ID NO: 6]
GCCGGGCCTCGAAACCCTT *
[SEQ ID NO: 1]
CCGGAGCTTTGGGAAGCCGp
A1B:
[SEQ ID NO: 7]
GCCGGGCCTCGCCGCCCTT *
[SEQ ID NO: 2]
CCGGAGCGGCGGGAAGCCGp
BA Adapter:
[SEQ ID NO: 10]
CGGCCTCGACGCCCTT *
[SEQ ID NO: 5]
GAGCTGCGGGAACGGCp

For example, using this approach in an inkjet synthesis system, only two jets are required. If the first nozzle is loaded with A0B and the second nozzle with A1B, the cassette of choice (A0B or A1B) is deposited, then the substrate is rinsed with buffer, then rinsed with a buffer solution comprising BA adapter, then rinsed with buffer to remove the BA adapter, then a second cassette of choice is added, and so on, until the desired sequence is reached.

In certain embodiments, the method of synthesizing DNA includes treating the DNA with a ligase and ATP. The topoisomerase only joins together one side of the DNA (the other is essentially nicked). The ligase would repair the nick and ensure that the topoisomerase itself doesn't recut the reaction product and cleave it. In some embodiments, ligase and ATP are provided in each cycle of addition. In other embodiments, ligase and ATP are provided after desired nucleotide sequence is obtained. In still other embodiments, the nick is not repaired. Single stranded DNA may be preferred as a final product. A single-stranded DNA (“top strand”) may be obtained by dehybridizing the double stranded DNA and removing the strand consisting of unligated oligomer fragments, i.e., the strand having nicks (“bottom strand”).

In some embodiments, the method comprises using a topoisomerase inhibitor to suppress binding and activity of free topoisomerase to the DNA oligomer. Suitable inhibitors include novobiocin and coumermycin. Note that complete inhibition is not desirable, as a low level of topoisomerase activity can help ‘relax’ coiled DNA, which is useful especially when synthesizing long DNA chains.

### “Ink” Media Compatible With Topoisomerase Ligation Reagents

Designing a carrier media or “ink” for delivery of topoisomerase ligation reagents presents significant technical challenges. First, the media must be compatible with the reaction: it must not denature the topoisomerase, it must allow relatively fast reaction kinetics for the ligation, and it must not damage the DNA. Second, the media must be compatible with the inkjet nozzles, e.g., it must allow formation of consistent droplets, quickly, reliably, and without causing blockage of the jets. Third, it must have physical properties that allow the reaction to proceed once the droplet is transmitted to the reaction surface. Viscosity, surface tension, density, and printhead dimensions affect not only the fluid flow, which is important for delivering the droplet, but also the forces on the enzyme. The topoisomerase activity may also be affected by the concentrations of reagents and ions and the pH, and the droplets must not spread or evaporate too quickly, as this too could affect the activity of the topoisomerase.

For printing using a piezoelectric nozzle, a somewhat viscous media is required, e.g., ca. 5-14 cP. Viscosity for this purpose is measured at room temperature (the inkjet printing experiments are also carried at room temperature without heating the ink or the substrate, although that would be possible as the topoisomerase enzyme is quite robust.) Viscosity is measured on a TA Instruments™ Discovery™ HR-30 Hybrid Rheometer in the Examples below or on an m-VROC viscometer from RheoSense. The media could for example include solvents such as glycerol, ethylene glycol, or diethylene glycol, as well as low molecular weight polymers, such as polyvinyl alcohol, polyethylene glycol, polypropylene glycol, sodium carboxymethyl cellulose (CMC), hydroxy ethyl cellulose, sodium alginate, hyaluronic acid, or carrageenan. In one embodiment, the media comprises PEG 8000, e.g., at concentrations of 10%-15%. For example, the buffer media may be 10% PEG 8000, 0.6M NaCl, 10 mM Tris pH 8.0.

In some embodiments, the buffer media may comprise a nonionic surfactant, e.g. Tween 20. For example the buffer media may comprise 10% PEG 8000, 0.6M NaCl, 10 mM Tris pH 8.0, and 0.1% Tween, e.g., Tween 20.

In some embodiments, the buffer media may use an organic salt, e.g., sodium acetate (NaOAc), in lieu of NaCl, e.g. the buffer media may comprise 12% PEG 8000, 0.6M NaOAc, 10 mM Tris pH 8.0, and 0.1% Tween. In alternative embodiments, the buffer media may use an organic ammonium salt, e.g., ammonium acetate (NH₄Ac), in lieu of NaCl, e.g., the buffer media may comprise 10% PEG 8000, 10% glycerol, 500 mM ammonium acetate, 20 mM Tris pH 8.0, 100 uM MgCl₂, and optionally ≤0.1% saturated, water-soluble inert dye for visualization.

Humectants, e.g., glycerol, ethylene glycol, or pentanediol may be added to slow evaporation, e.g., in an amount of 1 to 20%, e.g., 5% glycerol or 10% glycerol.

After each cassette addition, the substrate is washed with buffer to remove the reagents. The buffer may contain a non-ionic surfactant such as Tween (e.g., 1M NaCl/0.05% Tween) Wash Buffer, the washing may be repeated to ensure removal of all reagents, and a final wash with a buffer free of surfactant.

In an aspect, the invention provides a method (Method 1) of synthesizing a DNA polymer using topoisomerase-mediated ligation, comprising:
(i) reacting a double-stranded acceptor DNA attached to a substrate with a topoisomerase charged with a double-stranded DNA oligomer (i.e., oligomer covalently bound at the 3′ end of a strand to a topoisomerase),
   wherein a strand of the acceptor DNA has a 5′ overhang,
   wherein the oligomer optionally comprises an informational sequence, a topoisomerase recognition sequence, and 5′ overhangs on both strands,
   wherein the 5′ overhang of the strand of the oligomer that does not bear the topoisomerase (“bottom strand”) is complementary to the 5′ overhang of the acceptor DNA but is not complementary to the 5′ overhang of the strand bearing the topoisomerase (“top strand”) of the oligomer,
   wherein the 5′ end of the strand bearing the topoisomerase (“top strand”) of the oligomer and 5′ end of the acceptor DNA are not protected, e.g., not phosphorylated (i.e., 5′-OH), and
   wherein the topoisomerase charged with a double-stranded DNA oligomer is delivered to the location of the acceptor strand by a piezo-electric inkjet nozzle;
(ii) reacting the acceptor DNA thus extended with a topoisomerase charged with a further double-stranded DNA oligomer,
   wherein the further oligomer optionally comprises an informational sequence that is the same as or is different from any informational sequence in the oligomer of step (i), a topoisomerase recognition sequence, and 5′ overhangs on both strands, wherein the 5′ overhang of the strand of the further oligomer not bearing the topoisomerase (“bottom strand”) is complementary to the 5′ overhang of the extended acceptor DNA but is not complementary to the 5′ overhang of the strand of the further oligomer bearing the topoisomerase (“top strand”), and
   wherein the 5′ end of the strand bearing the topoisomerase (“top strand”) of the further oligomer is not protected, e.g., not phosphorylated (i.e., 5′-OH); and
(iii) repeating steps (i) and (ii) until the desired nucleotide sequence is obtained. For example, the invention provides:
   1.1. Method 1 comprising providing ligase and ATP to seal nicks in the DNA [NB: the topoisomerase ligation only ligates one strand].
   1.2. Method 1.1, wherein ligase and ATP is provided in step (i) and step (ii).
   1.3. Method 1.1, wherein ligase and ATP is provided after desired nucleotide sequence is obtained.
   1.4. Any foregoing method wherein the topoisomerase charged with a double-stranded DNA oligomer in step (i) and step (ii) is delivered in a buffer comprising a viscosity modifying agent, e.g., a reagent according to any of Reagent 1, et seq. below.
   1.5. Any foregoing method wherein the topoisomerase charged with a double-stranded DNA oligomer in step (i) and step (ii) is delivered in a buffer comprising a viscosity modifying agent, wherein the viscosity modifying agent is selected from polyethylene glycol (PEG), glycerol, sodium carboxymethylcellulose, and combinations thereof, e.g. PEG 8000 or a combination of PEG 8000 and glycerol.
   1.6. The foregoing method wherein the viscosity modifier comprises PEG 8000 at a concentration of 5%-15%, e.g. about 10%
   1.7. The foregoing method wherein the viscosity modifier further comprises glycerol, e.g. at a concentration of 5%-15%, e.g. about 10%
   1.8. Any foregoing method wherein the topoisomerase charged with a double-stranded DNA oligomer in step (i) and step (ii) is delivered in a buffer comprising a viscosity modifying agent, wherein the buffer further comprises a salt selected from NaCl, e.g., about 0.6M NaCl; NaOAc, e.g., about 0.6M NaOAc; and/or NH₄Ac, e.g., about 500 mM NH₄Ac.
   1.9. Any foregoing method wherein the topoisomerase charged with a double-stranded DNA oligomer in step (i) and step (ii) is delivered in a buffer comprising a viscosity modifying agent, wherein the buffer further comprises a nonionic surfactant, e.g., Tween.
   1.10. Any foregoing method wherein the topoisomerase charged with a double-stranded DNA oligomer in step (i) and step (ii) is delivered in a buffer comprising a viscosity modifying agent, wherein the buffer comprises (i) 10% PEG 8000, 0.6M NaCl, 10 mM Tris pH 8.0; (ii) 10% PEG 8000, 0.6M NaCl, 10 mM Tris pH 8.0, and 0.1% Tween; (iii) 12% PEG 8000, 0.6M NaOAc, 10 mM Tris pH 8.0, and 0.1% Tween; (iv) 10% PEG 8000, 10% glycerol, 500 mM NH₄Ac, 20 mM Tris pH 8.0, and 100 uM MgCl₂; or (v) 5% PEG 8000, 500 mM NH₄Ac, 20 mM Tris pH 8.0, and 100 uM MgCl₂.
   1.11. Any foregoing method further comprising one or more rinsing steps after step (i) and after step (ii), e.g., using a buffer solution.
   1.12. The foregoing method wherein the one or more rinsing steps after step (i) and after step (ii) comprise rinsing first with a buffer solution comprising surfactant, e.g., comprising 1M NaCl and 0.05% Tween, and then with a buffer solution that does not comprise surfactant; e.g., wherein the one or more rinsing steps after step (i) and after step (ii) comprise a first rinse with a buffer having 1M or more NaCl and optionally an anionic surfactant, e.g., sodium dodecyl sulphate (SDS) to denature any remaining enzyme, then rinsing with dilute surfactant-free buffer.
   1.13. Any foregoing method further comprising one or more rinsing steps after step (i) and after step (ii), wherein the one or more rinsing steps after step (i) and after step (ii) comprise rinsing first with a solution comprising surfactant, e.g., 1% SDS in water, and then with a buffer solution that does not comprise surfactant, e.g., 20 mM Tris pH 8.0; e.g., wherein the one or more rinsing steps after step (i) and after step (ii) comprise a first rinse with a solution comprising a surfactant, e.g., to denature any remaining enzyme, then rinsing with surfactant-free buffer, e.g., 20 mM Tris pH 8.0; optionally wherein the sample (and some or all of any associated substrate) is dried between rinsing steps.
   1.14. Any foregoing method wherein in step (ii) the topoisomerase charged with a further double-stranded DNA oligomer is delivered to the location of the acceptor strand by a piezo-electric inkjet nozzle.
   1.15. Any foregoing method other than the preceding method wherein in step (ii) the topoisomerase charged with a further double-stranded DNA oligomer is delivered to the location of the acceptor strand by puddling (i.e., fully washing, immersing, dipping, or covering) the substrate with a reagent comprising the topoisomerase charged with the further double-stranded DNA oligomer.
   1.16. Any foregoing method, wherein the topoisomerase-charged double-stranded DNA oligomer has a structure as follows:

5′-<overhang><Information Sequence><topo recognition sequence>
3′<-------------Complement-----------------------><overhang>-5′

wherein * is a topoisomerase covalently bound to the 3′ end of the top strand.
1.17. Any foregoing method, wherein the topoisomerase is selected from vaccinia topoisomerase I and SFV topoisomerase I, optionally wherein the topoisomerase is vaccinia topoisomerase I.
1.18. Any foregoing method, wherein the topoisomerase recognition sequence is 5′-(C/T)CCTT-3′ or 5′-CCCTG-3′, optionally wherein the topoisomerase recognition sequence is 5′-CCCTT-3′.
1.19. Any foregoing method, wherein the topoisomerase-charged double-stranded DNA oligomer has a structure as follows:

5′-<overhang><Information Sequence>CCCTT*
3′<----Complement----->GGGAA<overhang>-5′

wherein * is a topoisomerase covalently bound to the 3′ end of the top strand.
1.20. Any foregoing method, wherein the informational sequence of oligomers is selected from at least two different sequences, optionally wherein the informational sequence of the oligomers is selected from two different sequences, e.g., wherein one sequence corresponds to ‘0’ and the other to ‘1’ in a binary code.
1.21. Any foregoing method, wherein the informational sequence is a sequence of 3-12 nucleotides, e.g., about 8 nucleotides.
1.22. Any foregoing method, wherein the 5′ overhang sequence of the strand complementary to the strand bearing the topoisomerase of the oligomers (“bottom strand”) is selected from at least two different sequences, optionally wherein the 5′ overhang sequence of the bottom strand is selected from two different sequences.
1.23. Any foregoing method, wherein the 5′ overhang sequence of the strand bearing the topoisomerase of the oligomers (“top strand”) is selected from at least two different sequences, optionally wherein the 5′ overhang sequence of the strand bearing the topoisomerase of the oligomers (“top strand”) is selected from two different sequences.
1.24. Any foregoing method, wherein the 5′ overhangs of the oligomers are sequences of 2-6 nucleotides, optionally wherein the 5′ overhangs are sequences of 4 nucleotides.
1.25. Any foregoing method, wherein the oligomer is selected from four oligomers: A0B, B0A, A1B, B1A, wherein “A” or “B” on the left and right ends indicates the types of overhangs of oligomers and “0” or “1” indicates the binary code corresponding to the information sequence of oligomers.
1.26. Any foregoing method wherein the oligomer is selected from three oligomers: A0B, A1B, and BA, wherein “A” or “B” on the left and right ends indicates the types of overhangs of oligomers, “0” or “1” indicates the binary code corresponding to the information sequence of oligomers, and BA is an adapter oligomer, e.g., wherein the acceptor strand receives a topoisomerase-conjugated oligomer A0B or A1B, wherein “A” or “B” on the left and right ends indicates the types of overhangs of oligomers and “0” or “1” indicates the binary code corresponding to the information sequence of oligomers, and the acceptor strand is then adapted with adapter oligomer BA, which binds to the terminal A0B or A1B and allows the addition of a further A0B or A1B.
1.27. Any foregoing method, wherein the oligomer is selected from eight oligomers: A00B, A01B, A10B, A11B, B00A, B01A, B10A, and B11A, wherein “A” or “B” on the left and right ends indicates the types of overhangs of oligomers and “0” or “1” indicates the binary code corresponding to the information sequence of oligomers.
1.28. Any foregoing method comprising use of a topoisomerase inhibitor to suppress binding and activity of free topoisomerase to the DNA oligomer, optionally wherein the inhibitors is selected from novobiocin and coumermycin.
1.29. Any foregoing method, wherein the acceptor DNA is on a substrate or magnetic bead, where it can be selectively exposed to or removed from the reagents as required to provide the desired sequence.
1.30. Any foregoing method comprising alternate addition of informational oligonucleotides and adapter oligonucleotides, for example a method comprising:
(i) reacting a double-stranded acceptor DNA with topoisomerase-charged double-stranded DNA oligomer having a structure of Formula 1:

5′-HO-<Overhang A><Information Sequence><topo recognition>*
3′<----------------Complement-----------><Overhang A>P-5′

wherein * is a topoisomerase covalently bound to the 3′ end of the top strand;
wherein the Information Sequence may be varied, for example selected from two different sequences to provide a binary code in the DNA sequence synthesized;
wherein “topo recognition” is a topoisomerase recognition sequence, e.g., 5′-(C/T)CCTT-3′ or 5′-CCCTG-3′, for example 5′-CCCTT-3′;
wherein a strand of the acceptor DNA has a 5′ overhang which comprises a sequence complementary to Overhang A (Overhang B);
wherein Complement signifies a sequence which is complementary to “<Information Sequence 0 or 1><topo recognition>”;
wherein P is phosphate;
wherein the 5′ end of the acceptor DNA is not protected, e.g., not phosphorylated (i.e., 5′-OH); and
wherein Formula 1 may optionally comprise regions of one or more spacer nucleotides in addition to the regions specifically identified;
so that the double-stranded DNA oligomer extends the double-stranded acceptor DNA in the 3′ to 5′ direction, comprising an unprotected overhang (i.e., 5′-OH) which is Overhang A, and the topoisomerase is released;
(ii) reacting the acceptor DNA thus extended with a topoisomerase-charged double-stranded DNA oligomer having a structure of Formula 2 as follows:

5′-HO-<Overhang B><topo recognition>*
3′<--Complement---><Overhang B>P-5′

wherein * is a topoisomerase covalently bound to the 3′ end of the top strand;
wherein “topo recognition” is a topoisomerase recognition sequence, e.g., 5′-(C/T)CCTT-3′ or 5′-CCCTG-3′, for example 5′-CCCTT-3′;
wherein Overhang B is complementary to Overhang A above;
wherein Complement signifies a sequence which is complementary to “<topo recognition sequence>”;
wherein P is phosphate; and
wherein Formula 2 may optionally comprise regions of one or more spacer nucleotides in addition to the regions specifically identified;
so that the double-stranded DNA oligomer extends the double-stranded acceptor DNA in the 3′ to 5′ direction, with an unprotected overhang (i.e., 5′-OH) which is Overhang B, and the topoisomerase is released;
(iii) repeating steps (i) and (ii), varying the Information Sequence in the oligonucleotide sequence of step (i) as desired, until the desired nucleotide sequence is obtained.
   1.31. Method 1.27, comprising providing ligase and ATP to seal nicks in the DNA [NB: the topoisomerase ligation only ligates one strand], e.g., wherein ligase and ATP is provided in step (i) and step (ii) and/or wherein ligase and ATP is provided after desired nucleotide sequence is obtained.
   1.32. Any foregoing method wherein the topoisomerase is selected from vaccinia topoisomerase I and SFV topoisomerase I, optionally wherein the topoisomerase is vaccinia topoisomerase I.
   1.33. Any foregoing method wherein the topoisomerase recognition sequence is 5′-(C/T)CCTT-3′ or 5′-CCCTG-3′.
   1.34. Any foregoing method wherein the topoisomerase recognition sequence is 5′-CCCTT-3′.
   1.35. Any foregoing method wherein the Information Sequence in step (i) is selected from at least two different sequences, optionally wherein the Information Sequence is selected from two different sequences, e.g., wherein one sequence corresponds to ‘0’ and the other to ‘1’ in a binary code.
   1.36. Any foregoing method, wherein the Information Sequence is a sequence of 3-12 nucleotides, e.g., about 8 nucleotides.
   1.37. Any foregoing method, wherein the 5′ overhangs of the oligomers are sequences of 2-6 nucleotides, optionally wherein the 5′ overhangs are sequences of 4 nucleotides.
   1.38. Any foregoing method, comprising use of a topoisomerase inhibitor to suppress binding and activity of free topoisomerase to the DNA oligomer, optionally wherein the inhibitors is selected from novobiocin and coumermycin.
   1.39. Any foregoing method where, once the desired sequence is obtained the DNA is released from the substrate, e.g., by a cleaving reagent, e.g., an endonuclease specific for a site in the original acceptor strand, and the DNA is collected.
   1.40. Any foregoing method wherein the substrate is a silicon wafer.
   1.41. Any foregoing method wherein the double-stranded acceptor DNA attached to the substrate via a strain-promoted azide-alkyne cycloaddition (SPAAC).
   1.42. Any foregoing method wherein the double-stranded DNA acceptor is attached to phosphonate moiety via a residue of a SPAAC reaction, e.g., a reaction of an azide with azodibenzocyclooctyne (ADIBO) or dibenzocyclooctyne (DBCO), e.g., an attachment as follows:

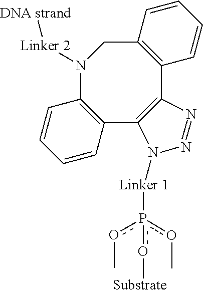

wherein the phosphonate moiety is attached to a metal oxide substrate, e.g., hafnium oxide or silica, and Linker 1 and Linker 2 are alkyl linkers optionally comprising one or more hydroxy, ether, ester, amine, or amide moieties, e.g., as depicted in e.g. as depicted in FIG. 4 or FIG. 26A.
   1.43. Any foregoing method wherein the substrate contains regions of DNA acceptor strands separated by hydrophobic regions, e,g hydrophobic regions coated with perfluorinated alkyl moieties, e.g., as depicted in FIGS. 23A and 23B.
   1.44. Any foregoing method wherein the strand density of DNA molecules in regions of DNA acceptor strands is 100-10,000 strands per um², e.g., 500-2500 strands per um².
   1.45. Any foregoing method wherein the substrate is substantially flat.
   1.46. Any foregoing method wherein the reagents comprising charged topoisomerase are selected from one or more of Reagent 1, et seq.

In another embodiment the disclosure provides a reagent (Reagent 1), e.g., for use in the above method, comprising a topoisomerase charged with a double-stranded DNA oligomer in a buffer solution comprising a viscosity modifying agent. For example, the disclosure provides
a) Reagent 1 wherein the solution has a viscosity of 5-14 cP.
b) Reagent 1 wherein the viscosity modifying agent comprises one or more of polyethylene glycol (PEG), e.g., PEG 8000, glycerol, and sodium carboxymethylcellulose.
c) Any foregoing reagent wherein the viscosity modifier comprises polyethylene glycol.
d) Any foregoing reagent wherein the viscosity modifier comprises glycerol.
e) Any foregoing reagent wherein the viscosity modifier comprises glycerol and polyethylene glycol.
f) Any foregoing reagent wherein the viscosity modifier comprises glycerol and PEG 8000.
g) Any foregoing reagent wherein the viscosity modifier comprises PEG 8000 at a concentration of 5%-15%, e.g., PEG 8000 at a concentration of about 10% or 12%, and glycerol at a concentration of 0% to 15%, e.g., about 10%
h) Any foregoing reagent wherein the viscosity modifier comprises PEG 8000 at a concentration of about 10% and glycerol at a concentration of about 10%.
i) Any foregoing reagent further comprising a salt, e.g., selected from NaCl, e.g., 0.6M NaCl; NaOAc, e.g., 0.6M NaOAc; and/or NH₄Ac, e.g., 500 mM NH₄Ac.
j) Any foregoing reagent wherein the buffer solution has a pKa of 7-9 at 25° C.
k) Any foregoing reagent wherein the buffer solution is a tris(hydroxymethyl)aminomethane (Tris) buffer solution, e.g., 5 mM to 30 mM Tris at pH 8, e.g. 10 mM Tris pH 8 or 20 mM Tris pH 8.
l) Any foregoing reagent further comprising an organic salt, e.g., an organic ammonium salt, e.g., ammonium acetate.
m) Any foregoing reagent comprising ammonium acetate, e.g., in a concentration of 200-800 mM, e.g. about 500 mM of ammonium acetate.
n) Any foregoing reagent further comprising a magnesium salt, e.g., magnesium chloride.
o) Any foregoing reagent further comprising a phosphatase, e.g., calf intestinal phosphatase (CIP).
p) Any foregoing reagent further comprising a nonionic surfactant, e.g., Tween, e.g., 0.1% Tween.
q) Any foregoing reagent selected from reagents comprising
   i. 10% PEG 8000, 0.6M NaCl, 10 mM Tris pH 8.0;
   ii. 10% PEG 8000, 0.6M NaCl, 10 mM Tris pH 8.0, and 0.1% Tween;
   iii. 12% PEG 8000, 0.6M NaOAc, 10 mM Tris pH 8.0, and 0.1% Tween;
   iv. 10% PEG 8000, 10% glycerol, 500 mM NH₄Ac, 20 mM Tris pH 8.0, and 100 uM MgCl₂; or
   v. 5% PEG 8000, 500 mM NH₄Ac, 20 mM Tris pH 8.0, and 100 uM MgCl₂.
r) Any foregoing reagent wherein the concentration of topoisomerase charged with a double-stranded DNA oligomer is 0.5-3 uM, e.g., about 1 uM or about 2.5 uM.
s) Any foregoing reagent selected from reagents comprising
   i. 1 uM topoisomerase charged with a double-stranded DNA oligomer, 0.6M Sodium Chloride, 10 mM Tris pH 8.0, 10% PEG 8000, 10% Glycerol, 0.1% Tween 20, 100 uM EDTA and 150 uM MgCl₂;
   ii. 2.5 uM topoisomerase charged with a double-stranded DNA oligomer, 10% PEG 8000, 10% glycerol, 500 mM NH₄Ac, 20 mM Tris pH 8.0, and 100 uM MgCl₂, optionally calf intestine phosphatase (CIP), optionally ≤0.1% saturated inert dye;
   iii. 2.5 uM topoisomerase charged with a double-stranded DNA oligomer, 500 mM Ammonium Acetate, 20 mM Tris HCl pH 8, 0.1 mM Magnesium Chloride, 10% w/v PEG 8000, 10% v/v Glycerol, <0.1% saturated inert dye (for visualization), 10 ug CIP per 2.5 nmol of topoisomerase (CIP=phosphatase, alkaline from bovine intestinal mucosa);
   iv. 0.5 uM topoisomerase charged with a double-stranded DNA oligomer, 5% PEG 8000, 500 mM NH₄Ac, 20 mM Tris pH 8.0, and 100 uM MgCl₂, optionally CIP, optionally ≤0.1% saturated inert dye; or
   v. 500 mM Ammonium Acetate, 20 mM Tris HCl pH 8, 0.1 mM Magnesium Chloride, 5% w/v PEG 8000, <0.1% saturated inert dye (for visualization), 0.5 uM charged Topoisomerase, 10 ug CIP per 2.5 nmol of topoisomerase (CIP=phosphatase, alkaline from bovine intestinal mucosa).

More generally, the disclosure provides methods of producing polymer memory strands using delivery of reagents using an ink jet, including but not restricted methods involving topoisomerase mediated ligation of DNA. For example, the disclosure provides methods (Method A) for writing, by at least one inkjet writing print head, a unique code to polymer memory strands dispensed on at least one writing spot on a wafer array, the head or nozzle writing the same code to a plurality of polymer memory strands dispensed on the at least one spot. For example, Method A comprises
A.1. Method A wherein the method comprises the following steps:
   a) loading the desired spot to be written with a starter polymer or DNA attached at one end to the desired spot;
   b) washing the surface of the spot;
   c) positioning an Add “0” or Add “1” inkjet nozzle having corresponding Add “0” and Add “1” reagents over the desired spot to be written corresponding to the unique code, wherein the Add “0” and Add “1” reagents comprise a monomer or oligomer encoding a “0” or “1”;
   d) causing the inkjet nozzle to release a droplet of the corresponding Add “0” or Add “1” reagent onto the spot, thereby writing a bit or portion of the unique code to the DNA or polymer memory strings (or strands) associated with the spot; and
   e) washing the surface of the spot.
A.2. Method A.1 further comprising the following steps:
   f) causing the inkjet nozzle to release a droplet of deblock/adapter reagent onto the spot;
   g) washing the surface of the spot; and
   h) repeating steps (c) through (g) until the unique code has been written in the memory string at the spot.
A.3. Method A.1 further comprising the following steps:
   f) applying to substrate an Add “0” or Add “1” reagent which will add only to polymer memory strands not modified by step c);
   g) repeating steps (b) through (f) until the unique code has been written in the memory string at the spot.
A.4. Any foregoing method comprising simultaneously writing, by a plurality of the writing print heads, unique codes to polymer memory strands dispensed on a plurality of writing spots on the wafer array.
A.5. Any foregoing method, wherein the polymer memory strands are DNA.
A.6. Any foregoing method, wherein the writing print head comprises a piezoelectric print head.
A.7. Any foregoing method, further comprising flowing a cleaving fluid over the spot thereby removing the memory strings from the spot and flowing the memory strings from the spot into a collection or storage container for later reading.
A.8. Method A which is a method for simultaneously writing, by a plurality of writing print heads, unique codes to polymer memory strands dispensed on a plurality of writing spots on a wafer array, each head or nozzle writing the same code to a plurality of DNA memory strands dispensed on a given spot, the method comprising:
   a. loading the desired spot to be written with starter polymer or DNA onto the desired spots; washing the surface of the wafer array;
   b. positioning an Add “0” or Add “1” inkjet nozzle having the corresponding Add “0” and Add “1” reagents over desired spot(s) to be written;
   c. causing the inkjet nozzle to release a droplet of the corresponding Add “0” or Add “1” reagent onto the spot, thereby writing a bit or code to the DNA or polymer memory strings (or strands) associated with the spot on the wafer array;
   d. washing the surface of the wafer array;
   e. causing the inkjet nozzle to release a droplet of deblock/adapter reagent onto the spot;
   f. washing the surface of the wafer array;
   g. when the code writing is complete for all the memory strings at all the spots on the wafer array;
   h. washing the surface of the wafer array with a cleaving fluid which removes the memory strings from the spots; and
   i. flowing the memory strings from the wafer array into a collection or storage container for later reading.
A.9. Any foregoing method wherein the at least one spot comprises a metal oxide surface which accepts phosphonate moieties that may be linked to DNA starter strands, wherein the spots are surrounded by hydrophobic regions.
A.10. The foregoing method wherein the metal oxide is HfO₂ and the hydrophobic regions comprise perfluoroalkyl moieties.
A.11. Any foregoing method, further comprising washing the wafer array with a preparation fluid before attaching the starter strands to the spots.
A.12. Any foregoing method comprising washing after each addition step, wherein the washing may be performed by flowing a washing fluid into an input port or manifold fluidically connected to one side of the wafer array causing the fluid to flow across the wafer surface and to exit an output port or manifold on an opposite side of the wafer.
A.13. Any foregoing method comprising washing after each addition step, wherein the washing may be performed by providing a washing print head with a nozzle which dispenses a predetermined amount of washing fluid to each desired spot on the wafer array surface.
A.14. Any foregoing method, wherein the starter strands or strings may be loaded and attached to the spots by providing a washing print head with a nozzle which dispenses a predetermined amount of starter strands in a fluid to each desired spot on the wafer array surface.
A.15. Any foregoing method, wherein the starter strings are attached to the spots, then dried and then rehydrated before use in the inkjet printer.
A.16. Any foregoing method, wherein, after writing the codes, the coded polymers attached to the spots on the array are then dried and stored, and then rehydrated and removed for reading or storing.
A.17. Any foregoing method, further comprising the step of, after writing is completed, unloading the polymer memory strands, e.g. coded DNA.

The system, computers, servers, devices and the like described herein have the necessary electronics, computer processing power, interfaces, memory, hardware, software, firmware, logic/state machines, databases, microprocessors, communication links (wired or wireless), displays or other visual or audio user interfaces, printing devices, and any other input/output interfaces, to provide the functions or achieve the results described herein. Except as otherwise explicitly or implicitly indicated herein, process or method steps described herein may be implemented within software modules (or computer programs) executed on one or more general-purpose computers. Specially designed hardware may alternatively be used to perform certain operations. Accordingly, any of the methods described herein may be performed by hardware, software, or any combination of these approaches. In addition, a computer-readable storage medium may store thereon instructions that when executed by a machine (such as a computer) result in performance according to any of the embodiments described herein.

In addition, computers or computer-based devices described herein may include any number of computing devices capable of performing the functions described herein, including but not limited to: tablets, laptop computers, desktop computers, smartphones, mobile communication devices, smart TVs, set-top boxes, e-readers/players, and the like.

Although the disclosure has been described herein using exemplary techniques, algorithms, or processes for implementing the present disclosure, it should be understood by those skilled in the art that other techniques, algorithms and processes or other combinations and sequences of the techniques, algorithms and processes described herein may be used or performed that achieve the same function(s) and result(s) described herein and which are included within the scope of the present disclosure.

Any process descriptions, steps, or blocks in process or logic flow diagrams provided herein indicate one potential implementation, do not imply a fixed order, and alternate implementations are included within the scope of the preferred embodiments of the systems and methods described herein in which functions or steps may be deleted or performed out of order from that shown or discussed, including substantially concurrently or in reverse order, depending on the functionality involved, as would be understood by those reasonably skilled in the art.

It should be understood that, unless otherwise explicitly or implicitly indicated herein, any of the features, functions, characteristics, alternatives or modifications described regarding a particular embodiment herein may also be applied, used, or incorporated with any other embodiment described herein. Also, the drawings herein are not drawn to scale, unless indicated otherwise.

Conditional language, such as, among others, “can,” “could,” “might,” or “may,” unless specifically stated otherwise, or otherwise understood within the context as used, is generally intended to convey that certain embodiments could include, but do not require, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment.

Although the invention has been described and illustrated with respect to exemplary embodiments thereof, the foregoing and various other additions and omissions may be made therein and thereto without departing from the spirit and scope of the present disclosure.

### Example 1—Printing With Topoisomerase-Based “Ink”

In this embodiment a Topoisomerase-based ink is prepared to contain 1 uM enzyme charged with DNA1 (top strand: 5′GCCGCTTGAAACCCTTCG3′ [SEQ ID NO:11], bottom strand 5′GCCGAAGGGTTTCAAG3′ [SEQ ID NO:12]), 0.6M Sodium Chloride, 10 mM Tris pH 8.0, 10% PEG 8000, 0.1% Tween 20, 100 uM EDTA and 150 uM MgCl₂. A Samba Dimatix Materials Cartridge from Fujifilm is filled with the Topoisomerase-based ink and the printing experiments were carried out on PixDro LP50 piezoelectric printer from SUSS MicroTech. The enzyme was jetted with a range of frequencies (1-10 kHz) and voltage pulses varying from 28-40V along a range of slew rates.

Images of Topoisomerase-based ink printed with 10 kHz frequency, 28V pulse with 40V/us slew rate on a glass slide and a clean 4 inch diameter silicon wafer are shown in FIG. 24A.

Topoisomerase enzyme bound to DNA1 and jetted with 30V pulse, at 10 KHz frequency is tested for ligation activity in a solution based assay, where the enzyme bound to DNA1 can perform ligation of DNA1 with free DNA2 (top strand: 5′CGGCAATCTGCACGTTAATATCGCAGGAATTCGTCAGCAG3′ [SEQ ID NO:13], bottom strand: 5′CTGCTGACGAATTCCTGCGATATTAACGTGCAGATT3′ [SEQ ID NO:14]). In this assay 25 nM of DNA2 is mixed with 250 nM topoisomerase bound to DNA1 (recovered after jetting through the Samba printhead) in 10 mM Tris pH 8.0, 10% PEG 8000, 0.1% Tween 20, 100 uM EDTA and 150 uM MgCl₂. 10 uL aliquotes of the mix are quenched with 1% SDS at time points 0, 20 seconds, 60 second and 5 minutes. Ligation of the two DNA pieces is monitored on a SeqStudio Genetic Analyzer System with SmartStart from ThermoFisher. Kinetic trace comparing ligation performance of a jetted and unjetted topoisomerase is shown in FIG. 24B.

### Example 2—Printing With Topoisomerase-Based “Ink”—Performance of Jetted vs. Unjetted Topoisomerase

In this embodiment a Topoisomerase-based ink is prepared to contain 1 uM enzyme charged with DNA1 (top strand: 5′GCCGCTTGAAACCCTTCG3′ [SEQ ID NO:11], bottom strand 5′GCCGAAGGGTTTCAAG3′ [SEQ ID NO:12]), 0.6M Sodium Chloride, 10 mM Tris pH 8.0, 10% PEG 8000, 10% Glycerol, 0.1% Tween 20, 100 uM EDTA and 150 uM MgCl₂. A Spectra printhead (SE 128-AA) from Fujifilm is filled with the Topoisomerase-based ink and the printing experiments were carried out on PixDro LP50 printer from SUSS MicroTech. The enzyme is jetted with a range of frequencies (1-10 kHz) and voltage pulses varying from 75-90V along a range of slew rates.

Images of Topoisomerase-based ink printed with 10 KHz frequency, 75V pulse a clean 4 inch diameter silicon wafer are shown in FIG. 25A

Topoisomerase enzyme bound to DNA1 and jetted with the Spectra printhead was tested for ligation activity in an assay described in Example 1. 10 uL aliquotes of the reaction mix were quenched with 1% SDS at time points 0, 20 seconds, 60 second and 5 minutes. Ligation of the two DNA pieces was monitored on a SeqStudio Genetic Analyzer System with SmartStart from ThermoFisher. Kinetic trace comparing ligation performance of a jetted and unjetted topoisomerase is shown in FIG. 25B.

### Example 3—Printing With Topoisomerase-Based “Ink” on a Patterned Wafer

In this embodiment a Topoisomerase-based ink is prepared to contain 1 uM enzyme charged with AB DNA cassette, 0.6M Sodium Chloride, 10 mM Tris pH 8.0, 10% PEG 8000, 10% Glycerol, 0.1% Tween 20, 100 uM EDTA and 150 uM MgCl₂. A Spectra printhead (SE 128-AA) from Fujifilm is filled with the ink formulation and the printing experiments were carried out on PixDro LP50 printer from SUSS MicroTech. The enzyme was jetted with 1 kHz frequency, and 75V pulse.

Topoisomerase ink is printed over a pattern of spots functionalized with strands of BA DNA1 (attached to the surface via SPAAC reaction click chemistry). Spots on the silicon wafer were 100 um wide and spaced at 100 dpi (center to center of the circular pattern). A cartoon of the pattern in shown in FIG. 26C. FIG. 26B illustrates the specifics of the attachment chemistry, where a patch of Hafnium Oxide is reacted with a carbon linker comprised of phosphonic acid moiety (specific reactivity for Hafnium Oxide) and a terminal azide that can react with a cycloalkyne moiety at the 5′ of the DNA2 strand, here a 5′-DBCO moiety.

Following a single addition of the Topoisomerase ink over the pattern, the enzyme is left to react with the surface bound BA-DNA1 for 5 minutes. The wafer is then removed from the printer, washed with 1M NaCl, 5 mM Tris pH 8.0, 0.05% Tween, before a complementary 1 uM topoisomerase solution functionalized with BA-DNA2 (identical sequence to BA-DNA1 but with no 5′-DBCO) is applied over the surface. The wafer is washed again with IM NaCl, 5 mM Tris pH 8.0, 0.05% Tween, dried, and positioned back on the LP50 stage. Following alignment of the wafer using the fiducial marker, the original ink containing 1 uM topoisomerase charged with AB-DNA cassette is printed over the spot pattern.

After 5 rounds of printing topoisomerase charged with AB-DNA followed by washing and by hand deposition of topoisomerase charged with BA-DNA2, the wafer was washed twice in 2× PBS buffer and air-dried. A portion of the wafer is treated with HiDi formamide reagent from ThermoFisher (Catalog number 4311320). Released DNA sequence is then analyzed on the SeqStudio Genetic Analyzer System with SmartStart from ThermoFisher, showing successful ligation.

We also performed a series of grafting/dehybridization experiments to determine the approximate concentrations of acceptor density on the HfOx surface, by introducing unique molecular identifiers (UMIs) into the system to help us quantitate number of molecules per nacket and PCR amplification biases/errors. The strand density per um² using different concentrations of DBCO-functionalized acceptor strands for grafting onto the azido-functionalized substrate is approximately as follows:

| | Graft concentration (nM) | molecules/um² |
|---|---|---|
| Sonicated: | 100 | 8504.0 |
| | 25 | 2893.1 |
| | 10 | 1102 |
| | 5 | 741 |
| | 1 | 344 |

### Example 4—Selection of Carrier Media for Topoisomerase

Various solvents are tested for viscosity and compatibility with the topoisomerase. Glycerol alone is found to lack adequate viscosity at lower concentrations, e.g., 4 cP at 30%, and to inhibit enzyme activity at higher concentrations, probably due to hydrogen bonding by the hydroxy groups. At lower concentrations, however, humectants such as glycerol, ethylene glycol, or pentanediol are useful to slow evaporation. Sugars such as sorbitol and trehalose are also not optimal as viscosity modifiers due to the need for high concentrations to provide adequate viscosity. Sodium carboxymethyl cellulose provides good viscosity at low concentrations and has fewer free hydroxy groups than other carbohydrates due to sodium substitution: 0.5% sodium carboxymethyl cellulose provides viscosity of 6 cp and does not significantly interfere with the topoisomerase activity (96% coupling efficiency after 5 minutes). Polyethylene glycol provides suitable viscosity, e.g., PEG 200 provides 7.6 cP at 40%, and PEG 8000 provides 6.5 cP at 10%.

PEG 8000 is selected for further evaluation. The stability of “charged” topoisomerase is measured by gel electrophoresis of topoisomerase linked to A0B cassettes stored up to 5 days at 4° C. in 15% PEG 8000, then run in 15% PEG 8000, 0.6M NaCl, and 10 mM Tris at pH 8.0. No DNA release is detected.

The efficiency of bit addition using various concentrations of PEG 8000 is measured in a five-minute reaction. There is no significant effect on coupling efficiency using 10% or 15% PEG 8000 stored at 4° C. or using 10% or 15% PEG 8000 following overnight incubation at room temperature. However, at 20% PEG 8000, the reaction efficiency drops significantly, to about 60% of control. Thus, while 20% PEG 8000 causes a decrease in topogation efficiency and slowing of the kinetics, 10-15% PEG (or 0.5% NaCMC) yield results comparable to the controls. Also, the addition of a non-ionic surfactant (Tween) does not have a significant effect on the reaction.

The impact of delivering the enzyme in a media of 10% PEG 8000, 0.6M NaCl, 10 mM Tris pH 8.0 (PEG INK) using an inkjet is then tested under various inkjet settings:
Topo-A0B in 0.6M NaCl, 10 mM Tris pH 8.0
Topo-A0B prepped in 10% PEG, 0.6M NaCl, 10 mM Tris pH 8.0
Topo-A0B jetted in 10% PEG INK with 28V pulse and 40V/us slew
Topo-A0B jetted in 10% PEG INK with 28V pulse and 80V/us slew
Topo-A0B jetted in 10% PEG INK with 40V pulse and 40V/us slew
Topo-A0B jetted in 10% PEG INK with 40V pulse and 80V/us slew
Topo-A0B in 10% PEG INK (not jetted, removed from the cartridge)
Topo-A0B jetted in 10% PEG INK with 28V pulse and 80V/us slew, 3000 Hz frequency
Topo-A0B jetted in 12.5% PEG INK with 34V pulse and 80V/us slew
2 uM Topo-A0B jetted in 10% PEG INK with 27V pulse and 80V/us slew
2 uM Topo-A0B jetted in 10% PEG INK with 40V pulse and 80V/us slew

At all settings tested, when measured using gel electrophoresis, while the higher viscosity buffer causes the bands to migrate slightly lower on the gel, there is no evidence of DNA discharge, indicating that the inkjet delivery did not affect the topoisomerase-DNA complex. Using the same array of inkjet settings, the efficiency of the topogation reaction is tested, and there is no evidence of decreased activity.

The initially developed media, comprising 10% PEG 8000, 0.6M NaCl, 10 mM Tris pH 8.0, and 0.1% Tween evaporates very quickly. Using a different salt, 0.6M NaOAc in place of 0.6M NaCl, reduces evaporation. It is also thought that the NaOAc may be less corrosive on the printer nozzles, as chloride can damage the piezoelectric film and affect printhead over longer term use. Further testing is carried out using a media comprising 12% PEG 8000, 0.6M NaOAc, 10 mM Tris pH 8.0, and 0.1% Tween, which provides similar good stability and high efficiency topogation.

To further reduce residue formation, we tried ammonium acetate rather than sodium acetate. Aqueous media comprising 500 mM Ammonium Acetate, 20 mM Tris HCl pH 8, 0.1 mM Magnesium Chloride, 10% w/v PEG 8000, 10% v/v Glycerol, <0.1% saturated inert dye (for visualization), 2.5 uM charged Topoisomerase, 10 ug CIP per 2.5 nmol of topoisomerase (CIP=phosphatase, alkaline from bovine intestinal mucosa) provides good stability and high efficiency topogation.

While a relatively viscous ink is preferred for printing, so as to avoid spraying or inaccurate delivery of ink to the desired spot, when the ink is used for “puddling,” i.e., pouring the ink over the substrate or immersing the substrate in the ink, an ink with lower viscosity is desirable. Aqueous media comprising 500 mM Ammonium Acetate, 20 mM Tris HCl pH 8, 0.1 mM Magnesium Chloride, 5% w/v PEG 8000, <0.1% saturated inert dye (for visualization), 0.5 uM charged Topoisomerase, 10 ug CIP per 2.5 nmol of topoisomerase (CIP=phosphatase, alkaline from bovine intestinal mucosa) provides good stability and high efficiency topogation using puddling.

For example, one “print/puddle” protocol, is as follows:
a. Preparing substrate: Sonication of the wafer before use (15 minutes of sonication in ethanol, rinse with isopropanol), followed by grafting of ADIBO or DBCO-functionalized DNA strands to azido-functionalized substrate, using 10 nM of acceptor in 2× PBS, for 30 min at RT (room temperature), followed by passivation using 2 uM DBCO-PEG7-OH in 2× PBS, for 30 min at RT.
b. Printing: Printheads—Spectra SL-128/80 AA Printhead. Head voltages: 75-90V. Head pulse lengths (us): 4. Perform fiducial/spot alignment for accuracy. Printhead storage buffer: 10% w/v PEG 8000, 10% v/v Glycerol in water Ink for LP-50-500 mM Ammonium Acetate, 20 mM Tris HCl pH 8, 0.1 mM Magnesium Chloride, 10% w/v PEG 8000, 10% v/v Glycerol, <0.1% saturated inert dye (for visualization), 2.5 uM charged Topoisomerase, 10 ug CIP per 2.5 nmol of topoisomerase (CIP=phosphatase, alkaline from bovine intestinal mucosa).
c. Puddling: The wafer is dipped in puddle ink-500 mM Ammonium Acetate, 20 mM Tris HCl pH 8, 0.1 mM Magnesium Chloride, 5% w/v PEG 8000, <0.1% saturated inert dye (for visualization), 0.5 uM charged Topoisomerase, 10 ug CIP per 2.5 nmol of topoisomerase (CIP=phosphatase, alkaline from bovine intestinal mucosa).
d. Wash protocol: 1% SDS in water (1-2 times, may partially dry wafer with airknife afterward); 20 mM Tris HCl pH 8 (3-8 times with airknife drying after some). Washing in this protocol is by dipping in the wash solution, but alternative washes, such as dipping in waterfall tanks (Mini Niagara), gentle spraying (La Rinsita) or gentle spraying is also feasible. The use of a denaturing agent such as SDS in the wash solution denatures any residual topoisomerase, thereby restricting any unwanted reaction, or alternative denaturing agents may be used.

What is claimed is:

1. A method for writing, by at least one inkjet writing print head, a unique code to polymer memory strands dispensed on at least one writing spot on a wafer array, the head or nozzle writing the same code to a plurality of polymer memory strands dispensed on the at least one spot.

2. The method of claim 1, comprising the following steps:
a) loading the desired spot to be written with a starter polymer or DNA attached at one end to the desired spot;
b) washing the surface of the spot;
c) positioning an Add “0” or Add “1” inkjet nozzle having corresponding Add “0” and Add “1” reagents over the desired spot to be written corresponding to the unique code, wherein the Add “0” and Add “1” reagents comprise a monomer or oligomer encoding a “0” or “1”;
d) causing the inkjet nozzle to release a droplet of the corresponding Add “0” or Add “1” reagent onto the spot, thereby writing a bit or portion of the unique code to the DNA or polymer memory strings (or strands) associated with the spot; and
e) washing the surface of the spot.

3. The method of claim 2 further comprising:
f) causing the inkjet nozzle to release a droplet of deblock/adapter reagent onto the spot;
g) washing the surface of the spot; and
h) repeating steps (c) through (g) until the unique code has been written in the memory string at the spot.

4. The method of claim 2 further comprising:
f) applying to substrate an Add “0” or Add “1” reagent which will add only to polymer memory strands not modified by step c);
g) repeating steps (b) through (f) until the unique code has been written in the memory string at the spot.

5. The method of claim 1 comprising simultaneously writing, by a plurality of the writing print heads, unique codes to polymer memory strands dispensed on a plurality of writing spots on the wafer array.

6. The method of claim 1, wherein the polymer memory strands are DNA.

7. The method of claim 1, wherein the writing print head comprises a piezoelectric print head.

8. The method of claim 1, further comprising flowing a cleaving fluid over the spot thereby removing the memory strings from the spot and flowing the memory strings from the spot into a collection or storage container for later reading.

9. The method of claim 1 for simultaneously writing, by a plurality of writing print heads, unique codes to polymer memory strands dispensed on a plurality of writing spots on a wafer array, each head or nozzle writing the same code to a plurality of DNA memory strands dispensed on a given spot, the method comprising:
loading the desired spot to be written with starter polymer or DNA onto the desired spots;
washing the surface of the wafer array;
positioning an Add “0” or Add “1” inkjet nozzle having the corresponding Add “0” and Add “1” reagents over desired spot(s) to be written;
causing the inkjet nozzle to release a droplet of the corresponding Add “0” or Add “1” reagent onto the spot, thereby writing a bit or code to the DNA or polymer memory strings (or strands) associated with the spot on the wafer array;
washing the surface of the wafer array;
causing the inkjet nozzle to release a droplet of deblock/adapter reagent onto the spot;
washing the surface of the wafer array;
when the code writing is complete for all the memory strings at all the spots on the wafer array;
washing the surface of the wafer array with a cleaving fluid which removes the memory strings from the spots; and
flowing the memory strings from the wafer array into a collection or storage container for later reading.

10. The method of claim 1 wherein the at least one spot comprises a metal oxide surface which accepts phosphonate moieties that may be linked to DNA starter strands, wherein the spots are surrounded by hydrophobic regions.

11. The method of claim 10 wherein the metal oxide is HfO₂ and the hydrophobic regions comprise perfluoroalkyl moieties.

12. The method of claim 9 wherein the washing may be performed by flowing a washing fluid into an input port or manifold fluidically connected to one side of the wafer array causing the fluid to flow across the wafer surface and to exit an output port or manifold on an opposite side of the wafer.

13. The method of claim 9 wherein the washing may be performed by providing a washing print head with a nozzle which dispenses a predetermined amount of washing fluid to each desired spot on the wafer array surface.

14. The method of claim 9 wherein the starter strands or strings may be loaded and attached to the spots by providing a washing print head with a nozzle which dispenses a predetermined amount of starter strands in a fluid to each desired spot on the wafer array surface.

15. The method of claim 9 wherein the starter strings are attached to the spots, then dried and then rehydrated before use in the inkjet printer.

16. The method of claim 9 wherein, after writing the codes, the coded polymers attached to the spots on the array are then dried and stored, and then rehydrated and removed for reading or storing.

17. The method of claim 8, wherein after writing is completed, unloading the coded polymer memory strands.

18. The method of claim 6 comprising synthesizing a DNA polymer using topoisomerase-mediated ligation, comprising:
(i) reacting a double-stranded acceptor DNA with a topoisomerase charged with a double-stranded DNA oligomer covalently bound to the topoisomerase),
wherein a strand of the acceptor DNA has a 5′ overhang,
wherein the oligomer optionally comprises an informational sequence, a topoisomerase recognition sequence, and 5′ overhangs on both strands,
wherein the 5′ overhang of the strand of the oligomer that does not bear the topoisomerase (“bottom strand”) is complementary to the 5′ overhang of the acceptor DNA but is not complementary to the 5′ overhang of the strand bearing the topoisomerase (“top strand”) of the oligomer,
wherein the 5′ end of the strand bearing the topoisomerase (“top strand”) of the oligomer and 5′ end of the acceptor DNA are not protected, e.g., not phosphorylated (i.e., 5′-OH), and
wherein the topoisomerase charged with a double-stranded DNA oligomer is delivered to the location of the acceptor strand by a piezo-electric inkjet nozzle;
(ii) reacting the acceptor DNA thus extended in step (i) with a topoisomerase charged with a further double-stranded DNA oligomer,
wherein the further oligomer optionally comprises an informational sequence that is the same as or is different from any informational sequence in the oligomer of step (i), a topoisomerase recognition sequence, and 5′ overhangs on both strands,
wherein the 5′ overhang of the strand of the further oligomer not bearing the topoisomerase (“bottom strand”) is complementary to the 5′ overhang of the extended acceptor DNA but is not complementary to the 5′ overhang of the strand of the further oligomer bearing the topoisomerase (“top strand”), and
wherein the 5′end of the strand bearing the topoisomerase (“top strand”) of the further oligomer is not protected, e.g., not phosphorylated (i.e., 5′-OH); and
(iii) repeating steps (i) and (ii) until the desired nucleotide sequence is obtained.

19. The method of claim 18 wherein there is a washing step after step (i) and after step (ii).

20. A reagent comprising a topoisomerase charged with a double-stranded DNA oligomer in a buffer solution comprising a viscosity modifying agent.
